SEQUENCE LISTING (166 sequences)

SEQ ID NO: 1 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 3 - protein, 4 aa, Artificial Sequence
  Description of Artificial Sequence: Synthetic peptide
TGGG

SEQ ID NO: 4 - protein, 11 aa, Artificial Sequence
  Description of Artificial Sequence: Synthetic peptide
TGGGGSGGGGS

SEQ ID NO: 5 - protein, 16 aa, Artificial Sequence
  Description of Artificial Sequence: Synthetic peptide
TGGGGSGGGGSGGGGS

SEQ ID NO: 6 - protein, 21 aa, Artificial Sequence
  Description of Artificial Sequence: Synthetic peptide
TGGGGSGGGGSGGGGSGGGGS

SEQ ID NO: 7 - protein, 10 aa, Artificial Sequence
  Description of Artificial Sequence: Synthetic peptide
TGGGPKSCDK

SEQ ID NO: 8 - DNA, 1248 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 415 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1284 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - protein, 427 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1299 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - protein, 432 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1269 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 422 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1266 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggacccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 17 - protein, 421 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 162 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 19 - protein, 5 aa, Artificial Sequence
  Description of Artificial Sequence: Synthetic peptide
GGGGS

SEQ ID NO: 20 - protein, 162 aa, Homo sapiens; identical to SEQ ID NO: 18

SEQ ID NO: 21 - protein, 10 aa, Artificial Sequence
  Description of Artificial Sequence: Synthetic peptide
GGGGSGGGGS

SEQ ID NO: 22 - protein, 22 aa, Unknown
  Description of Unknown: Native leader sequence
MGRGLLRGLWPLHIVLWTRIAS

SEQ ID NO: 23 - protein, 22 aa, Unknown
  Description of Unknown: Tissue plasminogen activator sequence
MDAMKRGLCCVLLLCGAVFVSP

SEQ ID NO: 24 - protein, 21 aa, Apis sp.
MKFLVNVALVFMVVYISYIYA

SEQ ID NO: 25 - protein, 26 aa, Artificial Sequence
  Description of Artificial Sequence: Synthetic peptide
TGGGGSGGGGSGGGGSGGGGSGGGGS

SEQ ID NO: 26 - protein, 31 aa, Artificial Sequence
  Description of Artificial Sequence: Synthetic polypeptide
TGGGGSGGGGSGGGGSGGGGSGGGGSGGGGS

SEQ ID NO: 27 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 28 - protein, 131 aa, Homo sapiens
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 29 - protein, 125 aa, Homo sapiens
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 30 - protein, 131 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 31 - protein, 125 aa, Homo sapiens
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 32 - protein, 119 aa, Homo sapiens
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 33 - protein, 156 aa, Homo sapiens
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - protein, 156 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 35 - protein, 150 aa, Homo sapiens
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 36 - protein, 137 aa, Artificial Sequence
  Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 37 - protein, 162 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 38 - protein, 131 aa, Artificial Sequence
  Description of Artificial Sequence: Synthetic polypeptide
TIPPHVQKSVNNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 39 - protein, 156 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 40 - protein, 36 aa, Artificial Sequence
  Description of Artificial Sequence: Synthetic polypeptide
TGGGGSGGGGSGGGGSGGGGSGGGGSGGGGSGGGGS

SEQ ID NO: 41 - protein, 36 aa, Homo sapiens
GRCKIRHIGSNNRLQRSTCQNTGWESAHVMKTPGFR

SEQ ID NO: 42 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 233 aa, Homo sapiens
GGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 437 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 442 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1314 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 47 - DNA, 1329 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggaagtggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 48 - skipped

SEQ ID NO: 49 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 51 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 52 - protein, 100 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 53 - protein, 512 aa, Artificial Sequence
  Description of Artificial Sequence: Synthetic polypeptide
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 54 - protein, 115 aa, Artificial Sequence
  Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 55 - protein, 100 aa, Artificial Sequence
  Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 56 - DNA, 1536 bp, Homo sapiens
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatc

SEQ ID NO: 57 - DNA, 345 bp, Homo sapiens
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccacc

SEQ ID NO: 58 - protein, 225 aa, Homo sapiens; identical to SEQ ID NO: 49

SEQ ID NO: 59 - protein, 223 aa, Homo sapiens; identical to SEQ ID NO: 42

SEQ ID NO: 60 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 61 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 62 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 3 aa, Artificial Sequence
  Description of Artificial Sequence: Synthetic peptide
GGG

SEQ ID NO: 64 - protein, 4 aa, Artificial Sequence
  Description of Artificial Sequence: Synthetic peptide
GGGG

SEQ ID NO: 65 - protein, 5 aa, Artificial Sequence
  Description of Artificial Sequence: Synthetic peptide
TGGGG

SEQ ID NO: 66 - protein, 5 aa, Artificial Sequence
  Description of Artificial Sequence: Synthetic peptide
SGGGG

SEQ ID NO: 67 - protein, 4 aa, Artificial Sequence
  Description of Artificial Sequence: Synthetic peptide
SGGG

SEQ ID NO: 68 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 69 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 70 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 72 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 228 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 75 - protein, 228 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWESNGQELPREKYLTWAPVLDSDGSFFLYSILRVAAEDWKKGDTFSCSVMHEALHNHYTQKSLDRSPGK

SEQ ID NO: 76 - protein, 261 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLEKELQALEKENAQLEWELQALEKELAQGAT

SEQ ID NO: 77 - protein, 261 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLKKKLQALKKKNAQLKWKLQALKKKLAQGAT

SEQ ID NO: 78 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTENQVSLWCLVKGFYPSDIAVEWESNGQPENNYKTTPPVLDSDGSFFLYSKLTVDKSRWQQGNVFSCSVMHEALHNHYTQDSLSLSPGK

SEQ ID NO: 79 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESRGQPENNYKTTPPVLDSRGSFFLVSKLTVDKSRWQQGNVFSCSVMHEALHNHYTQKSLSLSPGK

SEQ ID NO: 80 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 81 - skipped

SEQ ID NO: 82 - protein, 385 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 83 - DNA, 1158 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 84 - protein, 360 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 85 - protein, 432 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 86 - DNA, 1332 bp, Artificial Sequence
  Description of Artificial Sequence: Synthetic polynucleotide
atggatgcgatgaaacgcggcctgtgctgcgtgctgctgctgtgcggcgcggtgtttgtgagcccgggcgccaccattccgccgcatgtgcagaaaagcgatgtggaaatggaagcgcagaaagatgaaattatttgcccgagctgcaaccgcaccgcgcatccgctgcgccatattaacaacgatatgattgtgaccgataacaacggcgcggtgaaatttccgcagctgtgcaaattttgcgatgtgcgctttagcacctgcgataaccagaaaagctgcatgagcaactgcagcattaccagcatttgcgaaaaaccgcaggaagtgtgcgtggcggtgtggcgcaaaaacgatgaaaacattaccctggaaaccgtgtgccatgatccgaaactgccgtatcatgattttattctggaagatgcggcgagcccgaaatgcattatgaaagaaaaaaaaaaaccgggcgaaaccttttttatgtgcagctgcagcagcgatgaatgcaacgataacattatttttagcgaagaatataacaccagcaacccggataccggtggcggcggcagcggcggcggcggcagcggcggcggcggcagcggcggcggcggcagcacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcaacggccagccggaaaacaactatgataccaccccgccggtgctggatagcgatggcagcttttttctgtatagcgatctgaccgtggataaaagccgctggcagcagggcaacgtgtttagctgcagcgtgatgcatgaagcgctgcataaccattatacccagaaaagcctgagcctgagcccgggcgatgatgatgataaagcgcatcatcatcatcatcattaa

SEQ ID NO: 87 - protein, 408 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 88 - protein, 385 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1158 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaggttctggaggtggaggaagtggtggaggtggttctggaggtggtggaagtactcacacatgcccaccgtgcccagcacctgaactcctgggggggccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 90 - protein, 360 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 432 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - DNA, 1299 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 93 - protein, 408 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 410 aa, Artificial Sequence
  Description of Artificial Sequence: Synthetic polypeptide
GATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 409 aa, Artificial Sequence
  Description of Artificial Sequence: Synthetic polypeptide
ATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 408 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 407 aa, Artificial Sequence
  Description of Artificial Sequence: Synthetic polypeptide
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 406 aa, Artificial Sequence
  Description of Artificial Sequence: Synthetic polypeptide
PPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 405 aa, Artificial Sequence
  Description of Artificial Sequence: Synthetic polypeptide
PHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 404 aa, Artificial Sequence
  Description of Artificial Sequence: Synthetic polypeptide
HVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 102 - protein, 150 aa, Sus sp.
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 103 - protein, 150 aa, Mus sp.
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 104 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 105 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 106 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 107 - protein, 154 aa, Artificial Sequence
  Description of Artificial Sequence: Synthetic polypeptide
  MOD_RES (8)..(8): Thr, Ala or absent
  MOD_RES (121)..(121): Pro, Ala, Val or Met
MTAPWAAXLALLWGSLCAGSGRGEAETRECIYYNANWELERTNQSGLERLCEGEQDKRLHCYASWRNSSGTIELVKKGCWLDDFNCYDRQECVATEENPQVYFCCCEGNFCNERFTHLPEXGGPEVTYEPKPPTAPTLLTVLAYSLLPIGGLSM

SEQ ID NO: 108 - protein, 150 aa, Bos taurus
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 109 - protein, 107 aa, Artificial Sequence
  Description of Artificial Sequence: Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 110 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 111 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 112 - protein, 100 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 113 - DNA, 1539 bp, Homo sapiens
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 114 - DNA, 345 bp, Homo sapiens
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 115 - protein, 115 aa, Ovis aries
ILGRSETQECIFYNANWERDRTNRTGVESCYGDKDKRRHCFATWKNISGSIDIVKQGCWLDDINCYDRTDCIEKKDSPEVYFCCCEGNMCNERFSYFPEMEVTQPTSNPVTPKPP

SEQ ID NO: 116 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 117 - protein, 115 aa, Bos taurus
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 118 - protein, 115 aa, Tyto alba; identical to SEQ ID NO: 116

SEQ ID NO: 119 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 120 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 121 - protein, 767 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 122 - DNA, 2553 bp, Homo sapiens
atgacttcccattatgtgattgccatctttgccctgatgagctcctgtttagccactgcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgcagaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtgatgggcattgcgtttgcagcctttgtgatcggagcactcctgacgggggccttgtggtacatctattctcacacaggggagacagcaggaaggcagcaagtccccacctccccgccagcctcggaaaacagcagtgctgcccacagcatcggcagcacgcagagcacgccttgctccagcagcagcacggcc

SEQ ID NO: 123 - DNA, 2301 bp, Homo sapiens
ggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgcagaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtg

SEQ ID NO: 124 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 125 - protein, 766 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 126 - DNA, 2550 bp, Homo sapiens
atgacttcccattatgtgattgccatctttgccctgatgagctcctgtttagccactgcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtgatgggcattgcgtttgcagcctttgtgatcggagcactcctgacgggggccttgtggtacatctattctcacacaggggagacagcaggaaggcagcaagtccccacctccccgccagcctcggaaaacagcagtgctgcccacagcatcggcagcacgcagagcacgccttgctccagcagcagcacggcc

SEQ ID NO: 127 - DNA, 2298 bp, Homo sapiens
ggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtgcaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgcctgccctgcagaacccgcccatccggggaggggaaggccaaaatggaggccttccgtttcctttcccagatatttccaggagagtctggaatgaagagggagaagatgggctccctcggccaaaggaccctgtcattcccagcatacaactgtttcctggtctcagagagccagaagaggtgcaagggagcgtggatattgccctgtctgtcaaatgtgacaatgagaagatgatcgtggctgtagaaaaagattcttttcaggccagtggctactcggggatggacgtcaccctgttggatcctacctgcaaggccaagatgaatggcacacactttgttttggagtctcctctgaatggctgcggtactcggccccggtggtcagcccttgatggtgtggtctactataactccattgtgatacaggttccagcccttggggacagtagtggttggccagatggttatgaagatctggagtcaggtgataatggatttccgggagatatggatgaaggagatgcttccctgttcacccgacctgaaatcgtggtgtttaattgcagccttcagcaggtgaggaaccccagcagcttccaggaacagccccacggaaacatcaccttcaacatggagctatacaacactgacctctttttggtgccctcccagggcgtcttctctgtgccagagaatggacacgtttatgttgaggtatctgttactaaggctgaacaagaactgggatttgccatccaaacgtgctttatctctccatattcgaaccctgataggatgtctcattacaccattattgagaatatttgtcctaaagatgaatctgtgaaattctacagtcccaagagagtgcactttcctatcccgcaagctgacatggataagaagcgattcagctttgtcttcaagcctgtcttcaacacctcactgctctttctacagtgtgagctgacgctgtgtacgaagatggagaagcacccccagaagttgcctaagtgtgtgcctcctgacgaagcctgcacctcgctggacgcctcgataatctgggccatgatgcagaataagaagacgttcactaagccccttgctgtgatccaccatgaagcagaatctaaagaaaaaggtccaagcatgaaggaaccaaatccaatttctccaccaattttccatggtctggacaccctaaccgtg

SEQ ID NO: 128 - protein, 386 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 129 - DNA, 1161 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 130 - protein, 361 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 131 - protein, 432 aa, Artificial Sequence; identical to SEQ ID NO: 85
  Description of Artificial Sequence: Synthetic polypeptide

SEQ ID NO: 132 - DNA, 1332 bp, Artificial Sequence; identical to SEQ ID NO: 86
  Description of Artificial Sequence: Synthetic polynucleotide

SEQ ID NO: 133 - protein, 408 aa, Artificial Sequence; identical to SEQ ID NO: 87
  Description of Artificial Sequence: Synthetic polypeptide

SEQ ID NO: 134 - protein, 386 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - DNA, 1161 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 136 - protein, 361 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 432 aa, Artificial Sequence; identical to SEQ ID NO: 91
  Description of Artificial Sequence: Synthetic polypeptide

SEQ ID NO: 138 - DNA, 1299 bp, Artificial Sequence; identical to SEQ ID NO: 92
  Description of Artificial Sequence: Synthetic polynucleotide

SEQ ID NO: 139 - protein, 408 aa, Artificial Sequence; identical to SEQ ID NO: 93
  Description of Artificial Sequence: Synthetic polypeptide

SEQ ID NO: 140 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - DNA, 1114 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 143 - protein, 20 aa, Unknown
  Description of Unknown: Native ActRIIA leader sequence
MGAAAKLAFAVFLISCSSGA

SEQ ID NO: 144 - protein, 9 aa, Artificial Sequence
  Description of Artificial Sequence: Synthetic peptide
ILGRSETQE

SEQ ID NO: 145 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 148 - protein, 20 aa, Homo sapiens
MGAAAKLAFAVFLISCSSGA

SEQ ID NO: 149 - protein, 6 aa, Artificial Sequence
  Description of Artificial Sequence: Synthetic peptide
GRGEAE

SEQ ID NO: 150 - protein, 125 aa, Homo sapiens; identical to SEQ ID NO: 29

SEQ ID NO: 151 - protein, 17 aa, Artificial Sequence
  Description of Artificial Sequence: Synthetic peptide
NTSNPDTGGGTHTCPPC

SEQ ID NO: 152 - protein, 24 aa, Artificial Sequence
  Description of Artificial Sequence: Synthetic peptide
NTSNPDTGGGGSGGGGSTHTCPPC

SEQ ID NO: 153 - protein, 29 aa, Artificial Sequence
  Description of Artificial Sequence: Synthetic peptide
NTSNPDTGGGGSGGGGSGGGGSTHTCPPC

SEQ ID NO: 154 - protein, 34 aa, Artificial Sequence
  Description of Artificial Sequence: Synthetic polypeptide
NTSNPDTGGGGSGGGGSGGGGSGGGGSTHTCPPC

SEQ ID NO: 155 - protein, 23 aa, Artificial Sequence
  Description of Artificial Sequence: Synthetic peptide
NTSNPDTGGGPKSCDKTHTCPPC

SEQ ID NO: 156 - protein, 115 aa, Unknown
  Description of Unknown: Talpidae family peptide
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 157 - protein, 115 aa, Mus sp.
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 158 - protein, 10 aa, Artificial Sequence
  Description of Artificial Sequence: Synthetic peptide
  See specification as filed for detailed description of substitutions and preferred embodiments
GGGGSGGGGS

SEQ ID NO: 159 - protein, 15 aa, Artificial Sequence
  Description of Artificial Sequence: Synthetic peptide
  See specification as filed for detailed description of substitutions and preferred embodiments
GGGGSGGGGSGGGGS

SEQ ID NO: 160 - protein, 20 aa, Artificial Sequence
  Description of Artificial Sequence: Synthetic peptide
  See specification as filed for detailed description of substitutions and preferred embodiments
GGGGSGGGGSGGGGSGGGGS

SEQ ID NO: 161 - protein, 20 aa, Artificial Sequence
  Description of Artificial Sequence: Synthetic peptide
  MISC_FEATURE (1)..(20): This sequence may encompass 1-4 “Gly Gly Gly Gly Ser” repeating units
  See specification as filed for detailed description of substitutions and preferred embodiments
GGGGSGGGGSGGGGSGGGGS

SEQ ID NO: 162 - protein, 20 aa, Artificial Sequence
  Description of Artificial Sequence: Synthetic peptide
GGGGSGGGGSGGGGSGGGGS

SEQ ID NO: 163 - protein, 5 aa, Artificial Sequence
  Description of Artificial Sequence: Synthetic peptide
  See specification as filed for detailed description of substitutions and preferred embodiments
GGGGS

SEQ ID NO: 164 - protein, 20 aa, Artificial Sequence
  Description of Artificial Sequence: Synthetic peptide
  MISC_FEATURE (1)..(20): This sequence may encompass 1-4 “Gly Gly Gly Gly Ser” repeating units
  See specification as filed for detailed description of substitutions and preferred embodiments
GGGGSGGGGSGGGGSGGGGS

SEQ ID NO: 165 - protein, 5 aa, Artificial Sequence
  Description of Artificial Sequence: Synthetic peptide
GGGGS

SEQ ID NO: 166 - protein, 6 aa, Artificial Sequence
  Description of Artificial Sequence: Synthetic 6xHis tag
HHHHHH

(19) United States
(12) Patent Application Publication
(10) Pub. No.: US 2022/0089683 A1
(43) Pub. Date: Mar. 24, 2022

(54) BI-AND TRI-FUNCTIONAL FUSION PROTEINS AND USES THEREOF

(71) Applicant: Acceleron Pharma Inc., Cambridge, MA (US)

(72) Inventors: Ravindra Kumar, Acton, MA (US); Robert Scott Pearsall, North Reading, MA (US)

(21) Appl. No.: 17/252,525

(22) PCT Filed: Jun. 14, 2019
(86) PCT No.: PCT/US2019/037175

§ 371 Date: Dec. 15, 2020

Related U.S. Application Data
(60) Provisional application No. 62/685,747, filed on Jun. 15, 2018.

(51) Int. Cl. C07K 14/71; C07K 16/22; C07K 16/28; A61P 35/00

(52) U.S. Cl. CPC C07K 14/71; C07K 16/22; C07K 16/2863; A61K 2039/505; C07K 16/2827; A61P 35/00; C07K 2319/30; C07K 16/2818

(57) ABSTRACT

Disclosed herein are bi- and tri-functional fusion proteins comprising two or more of an activin antagonist domain, a TOPβ antagonist domain, and an immune checkpoint antagonist domain. In addition, the disclosure provides methods of treating cancer, a tumor, a pre-neoplastic disorder, a hyperproliferative disorder, or a dysplastic disorder comprising administering a bi- or tri-functional fusion protein comprising two or more of an activin antagonist domain, a TOPβ antagonist domain, and an immune checkpoint antagonist domain. Optionally, such methods further comprise administering an additional active agent or supportive therapy for treating the cancer, a tumor, a pre-neoplastic disorder, a hyperproliferative disorder, or a dysplastic disorder.

### CROSS-REFERENCE TO RELATED APPLICATIONS

This application claims the benefit of priority from U.S. Provisional Application No. 62/685,747, filed on Jun. 15, 2018. The foregoing application is incorporated herein by reference in its entirety.

### BACKGROUND OF THE INVENTION

In cancer treatment, it has long been recognized that chemotherapy is associated with high toxicity and can lead to emergence of resistant cancer cell variants. Even with targeted therapy against overexpressed or activated oncoproteins important for tumor survival and growth, cancer cells invariably mutate and adapt to reduce dependency on the targeted pathway, such as by utilizing a redundant pathway. Cancer immunotherapy is a new paradigm in cancer treatment that instead of targeting cancer cells, focuses on the activation of the immune system. Its principle is to rearm the host's immune response, especially the adaptive T cell response, to provide immune surveillance to kill the cancer cells, in particular, the minimal residual disease that has escaped other forms of treatment, hence achieving long-lasting protective immunity.

FDA approval of the anti-CTLA-4 antibody ipilimumab for the treatment of melanoma in 2011 ushered in a new era of cancer immunotherapy. The demonstration that anti-PD-1 or anti-PD-L1 therapy induced durable responses in melanoma, kidney, and lung cancer in clinical trials further signify its coming of age (Pardoll, D. M., Nat Immunol. 2012; 13:1129-32). However, ipilimumab therapy is limited by its toxicity profile, presumably because anti-CTLA-4 treatment, by interfering with the primary T cell inhibitory checkpoint, can lead to the generation of new autoreactive T cells. While inhibiting the PD-L1/PD-1 interaction results in dis-inhibiting existing chronic immune responses in exhausted T cells that are mostly antiviral or anticancer in nature (Wherry, E. J., Nat Immunol. 2011; 12:492-9), anti-PD-1 therapy can nevertheless sometimes result in potentially fatal lung-related autoimmune adverse events. Despite the promising clinical activities of anti-PD1 and anti-PD-L1 so far, increasing the therapeutic index, either by increasing therapeutic activity or decreasing toxicity, or both, remains a central goal in the development of immunotherapeutics.

Thus, there is still is a high unmet need for effective therapies, particularly therapies with low toxicity profiles for treating cancer in patients. Accordingly, it is an object of the present disclosure to provide compositions and improved methods for treating cancer in patients in need thereof.

### SUMMARY OF THE INVENTION

In part, the data presented herein demonstrates that activin antagonists (inhibitors) and TGFβ antagonists can be used alone or in combination to treat cancer. In particular, it was shown that treatment with an ActRIIA polypeptide, an ActRIIB polypeptide, or a pan-specific TGFβ antibody, separately, decreased tumor burden and increased survival time in a cancer model. Moreover, it was shown that an activin antagonist in combination with a TGFβ antagonist can be used to synergistically increase antitumor activity compared to the effects observed with either agent alone. In addition, the data indicate that efficacy of activin and TGFβ antagonist therapy is dependent on the immune system. Therefore, in part, the instant disclosure relates to the discovery that activin and TGFβ antagonists may be used as immunotherapeutics, particularly to treat a wide variety of cancers (e.g., cancers associated with immunosuppression and/or immune exhaustion). While not wishing to be bound by any particular theory, it is believed that such activin and TGFβ antagonist, alone or in combination, may be particularly useful in treating cancer when used in combination with an immune checkpoint antagonist (e.g., an antibody, or antigen-binding fragment thereof, that binds and inhibits one or more of (e.g., PD-1, PD-L1, CTLA4, BTLA, LAG3, TIM3, LAIR1, B7-DC, HVEM, TIM4, B7-H3, and/or B7-H4). Accordingly, the disclosure provides, in part, bi- and tri-functional fusion proteins comprising two or more domains selected from an activin antagonist domain, a TGFβ antagonist domain, and an immune checkpoint antagonist domain. The disclosure further provides, for example, methods of using such bi- and tri-functional fusion proteins to treat cancer, a tumor, a pre-neoplastic disorder, a hyperproliferative disorder, or a dysplastic disorder. Optionally, such methods further comprise administering to the patient an additional active agent or supportive therapy for treating the cancer, tumor, pre-neoplastic disorder, hyperproliferative disorder, or dysplastic disorder. As with other known immuno-oncology agents, the ability of such bi- and tri-functional fusion proteins to potentiate an immune response in a patient may have broader therapeutic implications outside the cancer field. For example, it has been proposed that immune potentiating agents may be useful in treating a wide variety of infectious diseases, particularly pathogenic agents which promote immunosuppression and/or immune exhaustion. Also, such immune potentiating agents may be useful in boosting the immunization efficacy of vaccines (e.g., infectious disease and cancer vaccines).

In certain aspects, an activin antagonist of the disclosure is an agent that inhibits activin (e.g. activin A, activin B, activin C, activin E, activin AB, and activin AE). Activin antagonists include, for example, polypeptides comprising an activin-binding domain (e.g., extracellular domains of ActRIIA and ActRIIB), antibodies or antigen-binding fragments thereof (e.g., anti-activin, anti-ActRIIA, and anti-ActRIIB antibodies or antigen-binding fragments thereof), small molecules, and polynucleotides. Effects on activin inhibition may be determined, for example, using a cell-based assay including those described herein (e.g., Smad signaling reporter assay). Therefore, in some embodiments, an activin antagonist of the disclosure may bind to activin. Ligand binding activity may be determined, for example, using a binding affinity assay including, for example, those described herein. In particular, the disclosure provides, in part, bi- and tri-functional fusion proteins that comprise an activin antagonist domain that binds to activin. For example, suitable activin antagonist domains include an activin-binding domain of an ActRIIA or ActRIIB polypeptide as well as antibodies, or antigen-binding fragments thereof, that bind to activin, ActRIIA, or ActRIIB. In some embodiments, an activin antagonist of the disclosure binds to at least activin A, activin B, activin AB, activin C, and/or activin E with a K_{D} of at least 1×10⁻⁸ M (e.g., at least 1×10⁻⁸ M, at least 1×10⁻⁹ M, at least 1×10⁻¹⁰ M, at least 1×10⁻¹¹ M, or at least 1×10⁻¹² M). In some embodiments, an activin antagonist, or combination of antagonists, of the disclosure binds to at least activin A and/or activin B with a K_{D} of at least 1×10⁻⁸ M (e.g., at least 1×10⁻⁸ M, at least 1×10⁻⁹ M, at least 1×10⁻¹⁰ M, at least 1×10⁻¹¹ M, or at least 1×10⁻¹² M). In some embodiments, an activin antagonist domain (e.g., ActRIIA and ActRIIB polypeptide domain) may further bind to one or more additional ligands including, for example, GDF8, GDF11, GDF3, BMP6, and BMP10. In some embodiments, a bi- or tri-functional fusion protein comprising an activin antagonist domain and one or more of a TGFβ antagonist domain or immune checkpoint antagonist domain may be used to treat cancer, a tumor, a pre-neoplastic disorder, a hyperproliferative disorder, or a dysplastic disorder. In some embodiments, a bi-functional fusion protein comprising a TGFβ antagonist domain and an immune checkpoint antagonist domain may be used in combination with an activin antagonist to treat cancer, a tumor, a pre-neoplastic disorder, a hyperproliferative disorder, or a dysplastic disorder.

In certain aspects, a TGFβ antagonist of the disclosure is an agent that inhibits TGFβ (e.g., TGFβ1, TGFβ2, and/or TGFβ3). TGFβ antagonists include, for example, polypeptides comprising a TGFβ-binding domain (e.g., extracellular domains of TβRII and betaglycan), antibodies or antigen-binding fragments thereof (e.g., anti-TGFβ, anti-TβRII, and anti-betaglycan antibodies or antigen-binding fragments thereof), small molecules, and polynucleotides. Effects on TGFβ inhibition may be determined, for example, using a cell-based assay including those described herein (e.g., Smad signaling reporter assay). Therefore, in some embodiments, an activin antagonist of the disclosure may bind to TGFβ. Ligand binding activity may be determined, for example, using a binding affinity assay including, for example, those described herein. In particular, the disclosure provides, in part, bi- and tri-functional fusion proteins that comprise a TGFβ antagonist domain that binds to TGFβ. For example, suitable activin antagonist domains include a TGFβ-binding domain of a TβRII or betaglycan polypeptide as well as antibodies, or antigen-binding fragments thereof, that bind to TGFβ, TβRII, or betaglycan. In some embodiments, an TGFβ antagonist of the disclosure binds to TGFβ1, TGFβ2, TGFβ 3 with a K_{D} of at least 1×10⁻⁸ M (e.g., at least 1×10⁻⁸ M, at least 1×10⁻⁹ M, at least 1×10⁻¹⁰ M, at least 1×10⁻¹¹ M, or at least 1×10⁻¹² M). In some embodiments, a TGFβ antagonist, or combination of antagonists, of the disclosure binds to TGFβ1 and TGFβ 3 with a K_{D} of at least 1×10⁻⁸ M (e.g., at least 1×10⁻⁸ M, at least 1×10⁻⁹ M, at least 1×10⁻¹⁰ M, at least 1×10⁻¹¹ M, or at least 1×10⁻¹² M), but does not substantially bind to TGFβ 2 (e.g., binds with a K_{D} of greater 1×10⁻⁷ M). In some embodiments, a bi- or tri-functional fusion protein comprising an TGFβ antagonist domain and one or more of an activin antagonist domain or immune checkpoint antagonist domain may be used to treat cancer, a tumor, a pre-neoplastic disorder, a hyperproliferative disorder, or a dysplastic disorder. In some embodiments, a bi-functional fusion protein comprising a activin antagonist domain and an immune checkpoint antagonist domain may be used in combination with a TGFβ antagonist to treat cancer, a tumor, a pre-neoplastic disorder, a hyperproliferative disorder, or a dysplastic disorder.

In certain aspects, an immune checkpoint antagonist of the disclosure is an agent that inhibits one or more immune checkpoint protein (e.g. PD-1, PD-L1, CTLA4, BTLA, LAG3, TIM3, LAIR1, B7-DC, HVEM, TIM4, B7-H3, and/or B7-H4). Immune checkpoint antagonists include, for example, polypeptides comprising an immune checkpoint-binding domain, antibodies or antigen-binding fragments thereof, small molecules, and polynucleotides. Effects on immune checkpoint inhibition may be determined, for example, using a cell-based assay including those described herein (e.g., Smad signaling reporter assay). Therefore, in some embodiments, an immune checkpoint antagonist of the disclosure may bind to immune checkpoint protein. Ligand binding activity may be determined, for example, using a binding affinity assay including, for example, those described herein. In particular, the disclosure provides, in part, bi- and tri-functional fusion proteins that comprise an immune checkpoint antagonist domain that binds to immune checkpoint protein. For example, suitable immune checkpoint antagonist domains include antibodies, or antigen-binding fragments thereof, that bind to one or more of PD-1, PD-L1, CTLA4, BTLA, LAG3, TIM3, LAIR1, B7-DC, HVEM, TIM4, B7-H3, and/or B7-H4. In some embodiments, an immune checkpoint antagonist of the disclosure binds to PD-1, PD-L1, CTLA4, BTLA, LAG3, TIM3, LAIR1, B7-DC, HVEM, TIM4, B7-H3, and/or B7-H4 with a K_{D} of at least 1×10⁻⁸ M (e.g., at least 1×10⁻⁸ M, at least 1×10⁻⁹ M, at least 1×10⁻¹⁰ M, at least 1×10⁻¹¹ M, or at least 1×10⁻¹² M). In some embodiments, an immune checkpoint antagonist, or combination of antagonists, of the disclosure binds to PD-1, PD-L1, or CTLA4 with a K_{D} of at least 1×10⁻⁸ M (e.g., at least 1×10⁻⁸ M, at least 1×10⁻⁹ M, at least 1×10⁻¹⁰ M, at least 1×10⁻¹¹ M, or at least 1×10⁻¹² M). In some embodiments, a bi- or tri-functional fusion protein comprising an immune checkpoint antagonist domain and one or more of a TGFβ antagonist domain or activin antagonist domain may be used to treat cancer, a tumor, a pre-neoplastic disorder, a hyperproliferative disorder, or a dysplastic disorder. In some embodiments, a bi-functional fusion protein comprising a TGFβ antagonist domain and an activin antagonist domain may be used in combination with an immune checkpoint antagonist to treat cancer, a tumor, a pre-neoplastic disorder, a hyperproliferative disorder, or a dysplastic disorder.

In some embodiments, the disclosure provides for a fusion protein comprising two or more domains selected from an activin antagonist domain, a TGFβ antagonist domain, and an immune checkpoint antagonist domain. In some embodiments, the protein comprises an activin antagonist domain and a TGFβ antagonist domain. In some embodiments, the protein comprises an activin antagonist domain and an immune checkpoint antagonist domain. In some embodiments, the protein comprises a TGFβ antagonist domain and an immune checkpoint antagonist domain. In some embodiments, the protein comprises an activin antagonist domain, a TGFβ antagonist domain, and an immune checkpoint antagonist domain. In some embodiments, the fusion protein further comprises a polypeptide domain that is heterologous to the activin antagonist domain, TGFβ antagonist domain, and/or immune checkpoint antagonist domain. In some embodiments, the fusion protein further comprises a linker domain. In some embodiments, the domains of the fusion protein are arranged in an order selected from the group consisting of: a) A-X-T; b) A-X-I; c) T-X-I; d) A-X-H-X-T; e) A-X-H-X-I; f) I-X-H-X-T; g) A-X-T-X-I; h) T-X-A-X-I; i) A-X-I-X-T; j) A-X-T-H-X-I; k) T-X-A-H-X-I; l) A-X-I-H-X-T; m) A-X-H-T-X-I; n) T-X-H-A-X-I; and o) A-X-H-I-X-T, wherein (i) “A” corresponds to an activin antagonist domain; (ii) “T” corresponds to a TGFβ antagonist domain; (iii) “I” corresponds to an immune checkpoint antagonist domain; (iv) “H” corresponds to a polypeptide domain that is heterologous to the activin antagonist domain, TGFβ antagonist domain, and immune checkpoint antagonist domains; and (v) “X” corresponds to an optional linker domain; and wherein the arrangement of the domains is either N-terminus to C-terminus or C-terminus to N-terminus.

In some embodiments, the disclosure provides for a homodimer comprising any of the fusion proteins disclosed herein.

In some embodiments, the disclosure provides for a heterodimer comprising two or more polypeptide domains selected from an activin antagonist domain, a TGFβ antagonist domain, and an immune checkpoint antagonist domain.

In some embodiments, the heterodimer comprises two polypeptides selected from: a) A-X-T; b) A-X-I; c) T-X-I; d) A-X-H-X-T; e) A-X-H-X-I; f) I-X-H-X-T; g) A-X-T-X-I; h) T-X-A-X-I; i) A-X-I-X-T; j) A-X-T-H-X-I; k) T-X-A-H-X-I; l) A-X-I-H-X-T; m) A-X-H-T-X-I; n) T-X-H-A-X-I; and o) A-X-H-I-X-T, wherein (i) “A” corresponds to an activin antagonist domain; (ii) “T” corresponds to a TGFβ antagonist domain; (iii) “I” corresponds to an immune checkpoint antagonist domain; (iv) “H” corresponds to a polypeptide domain that is heterologous to the activin antagonist domain, TGFβ antagonist domain, and immune checkpoint antagonist domains; and (v) “X” corresponds to an optional linker domain; and wherein the arrangement of the domains is either N-terminus to C-terminus or C-terminus to N-terminus.

In some embodiments, the disclosure provides for a heterodimer comprising: a) a first polypeptide comprising an immune checkpoint antagonist domain and a TGFβ antagonist domain; and b) a second polypeptide comprising an activin antagonist domain. In some embodiments, the second polypeptide further comprises an immune checkpoint antagonist domain.

In some embodiments, the disclosure provides for a heterodimer comprising: a) a first polypeptide comprising a TGFβ antagonist domain and an activin antagonist domain; and b) a second polypeptide comprising an immune checkpoint antagonist domain. In some embodiments, the second further comprise a TGFβ antagonist domain. In some embodiments, the second further comprise an activin antagonist domain. In some embodiments, the heterodimer further comprises one or more polypeptide domains that are heterologous to the activin antagonist domain, TGFβ antagonist domain, and/or immune checkpoint antagonist domain. In some embodiments, the heterodimer further comprises one or more linker domains. In some embodiments, the activin antagonist domain is an ActRIIA polypeptide. In some embodiments, the ActRIIA polypeptide is selected from the group consisting of: a) a polypeptide comprising an amino acid sequence that is at least 75% identical to a sequence beginning at any one of positions 21 to 30 of SEQ ID NO: 110, and ending at any one of positions 110 to 135 of SEQ ID NO: 110; b) a polypeptide comprising an amino acid sequence that is at least 75% identical to a sequence beginning at position 21 of SEQ ID NO: 110, and ending at position 135 of SEQ ID NO: 110; c) a polypeptide comprising an amino acid sequence that is at least 75% identical to a sequence beginning at position 30 of SEQ ID NO: 110, and ending at position 110 of SEQ ID NO: 110; d) a polypeptide comprising an amino acid sequence that is at least 75% identical to SEQ ID NO: 111; and e) a polypeptide comprising an amino acid sequence that is at least 75% identical to SEQ ID NO: 112. In some embodiments, the ActRIIB polypeptide binds to activin. In some embodiments, the ActRIIB polypeptide binds to activin A. In some embodiments, the ActRIIB polypeptide binds to activin B. In some embodiments, the ActRIIB polypeptide further binds to GDF8 and/or GDF11. In some embodiments, the ActRIIB polypeptide inhibits activin A and/or activin B signaling as determined using a reporter gene assay. In some embodiments, the ActRIIB polypeptide further inhibits GDF8 and/or GDF11 signaling as determined using a reporter gene assay. In some embodiments, the activin antagonist domain is an ActRIIB polypeptide. In some embodiments, the ActRIIB polypeptide is selected from the group consisting of: a) a polypeptide comprising an amino acid sequence that is at least 75% identical to a sequence beginning at any one of positions 20 to 29 of SEQ ID NO: 50, and ending at any one of positions 109 to 134 of SEQ ID NO: 50; b) a polypeptide comprising an amino acid sequence that is at least 75% identical to a sequence beginning at position 20 of SEQ ID NO: 50, and ending at position 134 of SEQ ID NO: 50; c) a polypeptide comprising an amino acid sequence that is at least 75% identical to a sequence beginning at position 29 of SEQ ID NO: 50, and ending at position 109 of SEQ ID NO: 50; d) a polypeptide comprising an amino acid sequence that is at least 75% identical to SEQ ID NO: 51; e) a polypeptide comprising an amino acid sequence that is at least 75% identical to SEQ ID NO: 52; f) a polypeptide comprising an amino acid sequence that is at least 75% identical to SEQ ID NO: 54; and g) a polypeptide comprising an amino acid sequence that is at least 75% identical to SEQ ID NO: 55. In some embodiments, the ActRIIB polypeptide binds to activin. In some embodiments, the ActRIIB polypeptide binds to activin A. In some embodiments, the ActRIIB polypeptide binds to activin B. In some embodiments, the ActRIIB polypeptide further binds to GDF8 and/or GDF11. In some embodiments, the ActRIIB polypeptide inhibits activin A and/or activin B signaling as determined using a reporter gene assay. In some embodiments, the ActRIIB polypeptide further inhibits GDF8 and/or GDF11 signaling as determined using a reporter gene assay. In some embodiments, the activin antagonist domain is an antibody, or antigen-binding fragment thereof, that binds to activin. In some embodiments, the antibody, or antigen-binding fragment thereof, binds to activin A. In some embodiments, the antibody, or antigen-binding fragment thereof, binds to activin B. In some embodiments, the antibody inhibits activin A and/or activin B signaling as determined using a reporter gene assay. In some embodiments, the activin antagonist domain is an antibody, or antigen-binding fragment thereof, that binds to an ActRII receptor. In some embodiments, the antibody, or antigen-binding fragment thereof, binds to ActRIIA. In some embodiments, the antibody, or antigen-binding fragment thereof, binds to ActRIIB. In some embodiments, the antibody, or antigen-binding fragment thereof, inhibits activin-ActRIIA and/or activin-ActRIIB signaling as determined using a reporter gene assay. In some embodiments, the antibody, or antigen-binding fragment thereof is bimagrumab, or an antigen-binding fragment thereof. In some embodiments, the TGFβ antagonist domain is a TβRII polypeptide. In some embodiments, the TβRII polypeptide is selected from the group consisting of: a) a polypeptide comprising an amino acid sequence that is at least 75% identical to a sequence beginning at any one of positions 23 to 35 of SEQ ID NO: 1, and ending at any one of positions 153 to 159 of SEQ ID NO: 1; b) a polypeptide comprising an amino acid sequence that is at least 75% identical to a sequence beginning at position 23 of SEQ ID NO: 1, and ending at position 159 of SEQ ID NO: 1; c) a polypeptide comprising an amino acid sequence that is at least 75% identical to a sequence beginning at position 35 of SEQ ID NO: 1, and ending at position 153 of SEQ ID NO: 1; d) a polypeptide comprising an amino acid sequence that is at least 75% identical to a sequence beginning at any one of positions 23 to 60 of SEQ ID NO: 2, and ending at any one of positions 178 to 184 of SEQ ID NO: 2; e) a polypeptide comprising an amino acid sequence that is at least 75% identical to a sequence beginning at position 23 of SEQ ID NO: 2, and ending at position 184 of SEQ ID NO: 2; f) a polypeptide comprising an amino acid sequence that is at least 75% identical to a sequence beginning at position 60 of SEQ ID NO: 2, and ending at position 178 of SEQ ID NO: 2; g) a polypeptide comprising an amino acid sequence that is at least 75% identical to SEQ ID NO: 18; h) a polypeptide comprising an amino acid sequence that is at least 75% identical to SEQ ID NO: 27; i) a polypeptide comprising an amino acid sequence that is at least 75% identical to SEQ ID NO: 20; and j) a polypeptide comprising an amino acid sequence that is at least 75% identical to any one of SEQ ID NOs: 28, 29, 30, 31, 32, 33, 34, 35, 36, 37, 38; and 39. In some embodiments, the polypeptide binds to TGFβ3. In some embodiments, the polypeptide binds to TGFβ1. In some embodiments, the polypeptide binds to TGFβ3. In some embodiments, the polypeptide inhibits TGFβ1 and/or TGFβ3 signaling as determined using a reporter gene assay. In some embodiments, the TGFβ antagonist domain is a betaglycan polypeptide. In some embodiments, the betaglycan polypeptide is selected from the group consisting of: a) a polypeptide comprising an amino acid sequence that is at least 75% identical to a sequence beginning at any one of positions 21 to 28 of SEQ ID NO: 120, and ending at any one of positions 381 to 787 of SEQ ID NO: 120; b) a polypeptide comprising an amino acid sequence that is at least 75% identical to a sequence beginning at position 21 of SEQ ID NO: 120, and ending at position 787 of SEQ ID NO: 120; c) a polypeptide comprising an amino acid sequence that is at least 75% identical to a sequence beginning at position 28 of SEQ ID NO: 120, and ending at position 381 of SEQ ID NO: 120; d) a polypeptide comprising an amino acid sequence that is at least 75% identical to SEQ ID NO: 121; and e) a polypeptide comprising an amino acid sequence that is at least 75% identical to SEQ ID NO: 125. In some embodiments, the polypeptide binds to TGFβ. In some embodiments, the polypeptide binds to TGFβ1. In some embodiments, the polypeptide binds to TGFβ2. In some embodiments, the polypeptide binds to TGFβ3. In some embodiments, the polypeptide inhibits TGFβ1, TGFβ2, and/or TGFβ3 signaling as determined using a reporter gene assay. In some embodiments, the TGFβ antagonist domain is an antibody, or antigen-binding fragment thereof, that binds to TGFβ. In some embodiments, the antibody, or antigen-binding fragment thereof, binds to TGFβ1. In some embodiments, the antibody, or antigen-binding fragment thereof, binds to TGFβ2. In some embodiments, the antibody, or antigen-binding fragment thereof, binds to TGFβ3. In some embodiments, the antibody, or antigen-binding fragment thereof is selected from the antibodies: fresolimumab, metelimumab, Lily21D1, LilyDM4, XOMA089, and XOMA681, or an antigen-binding fragment thereof. In some embodiments, the antibody inhibits TGFβ1, TGFβ2, and/or TGFβ3 signaling as determined using a reporter gene assay. In some embodiments, the heterologous portion comprises a first or second member of an interaction pair. In some embodiments, the heterologous portion comprises one or more amino acid modifications that promotes heterodimer formation. In some embodiments, the heterologous portion is an immunoglobulin Fc domain. In some embodiments, the immunoglobulin Fc domain is a human immunoglobulin Fc domain. In some embodiments, the immunoglobulin Fc domain is an immunoglobulin G1Fc domain. In some embodiments, the linker is between 10 and 25 amino acids in length. In some embodiments, the linker comprises an amino acid sequence selected from: a) (GGGGS)ₙ, wherein n=≥2; b) (GGGGS)ₙ, wherein n=≥3; c) (GGGGS)ₙ, wherein n=≥4; and d) the amino acid sequence of any one of SEQ ID Nos: 4-7, 19, 21, 25, 26, 40, and 63-67. In some embodiments, the linker comprises (GGGGS)ₙ, wherein n≠≥5. In some embodiments, the linker comprises (GGGGS)ₙ, wherein n≠≥5. In some embodiments, the fusion protein, homodimer or heterodimer comprises one or more modified amino acid residues selected from: a glycosylated amino acid, a PEGylated amino acid, a farnesylated amino acid, an acetylated amino acid, a biotinylated amino acid, and an amino acid conjugated to a lipid moiety. In some embodiments, the fusion protein, homodimer or heterodimer is glycosylated. In some embodiments, the fusion protein, homodimer or heterodimer has a glycosylation pattern characteristic of expression of the polypeptide in CHO cells. In some embodiments, the fusion protein, homodimer or heterodimer is isolated. In some embodiments, the fusion protein, homodimer or heterodimer is recombinant. In some embodiments, the immune checkpoint antagonist domain inhibits one or more of PD-1, PD-L1, CTLA4, BTLA, LAG3, TIM3, LAIR1, B7-DC, HVEM, TIM4, B7-H3, and/or B7-H4. In some embodiments, the immune checkpoint antagonist domain inhibits PD-1. In some embodiments, the immune checkpoint antagonist domain inhibits PD-L1. In some embodiments, the immune checkpoint antagonist domain inhibits CTLA-4. In some embodiments, the immune checkpoint antagonist domain is an antibody, or antigen-binding fragment thereof, that binds to one or more of PD-1, PD-L1, CTLA4, BTLA, LAG3, TIM3, LAIR1, B7-DC, HVEM, TIM4, B7-H3, and/or B7-H4. In some embodiments, immune checkpoint antagonist domain is an antibody, or antigen-binding fragment thereof, that binds to PD-1. In some embodiments, the immune checkpoint antagonist domain is an antibody, or antigen-binding fragment thereof, that binds to PD-L1. In some embodiments, the immune checkpoint antagonist domain is an antibody, or antigen-binding fragment thereof, that binds to CTLA4. In some embodiments, the immune checkpoint antagonist domain is an antibody selected from ipilimumab, nivolumab, pembrolizumab, atezolizumab, avelumab, and durvalumab, or antigen-binding fragment thereof.

In some embodiments, the disclosure provides for a pharmaceutical preparation comprising any of the fusion proteins, homodimers or heterodimers disclosed herein and a pharmaceutically acceptable excipient.

In some embodiments, the disclosure provides for an isolated polynucleotide comprising a coding sequence for any of the fusion proteins, homodimers or heterodimers disclosed herein.

In some embodiments, the disclosure provides for a recombinant polynucleotide comprising a promotor sequence operably linked to any of the polynucleotides disclosed herein.

In some embodiments, the disclosure provides for a cell comprising any of the polynucleotides disclosed herein. In some embodiments, the cell is a CHO cell.

In some embodiments, the disclosure provides for a method of making a fusion protein, homodimer or heterodimer comprising two or more domains selected from an activin antagonist domain, a TGFβ antagonist domain comprising culturing a cell under conditions suitable for expression of any of the polynucleotides disclosed herein.

In some embodiments, the disclosure provides for a method of treating cancer, a tumor, a pre-neoplastic disorder, a hyperproliferative disorder, or a dysplastic disorder comprising administering to a patient in need thereof an effective amount of one or more of any of the fusion proteins, homodimers, heterodimers or pharmaceutical preparations disclosed herein. In some embodiments, the cancer, tumor, pre-neoplastic disorder, hyperproliferative disorder, or dysplastic disorder is selected from the group consisting of: a hematopoietic tumor of lymphoid or myeloid lineage tumor of mesenchymal origin such as a fibrosarcoma or rhabdomyosarcoma, melanoma, intraocular melanoma, nonmelanoma skin cancer, teratocarci-noma, neuroblastoma, glioma, brain stem glioma, visual pathway and hypothalamic glioma, oligodendroglioma, adenocarcinoma, papillary adenocarcinomas, cystadenocarcinoma, carcinoma, non-small lung cell carcinoma, hepatoma, hepatocellular carcinoma, endometrial cancer or uterine carcinoma, salivary gland carcinoma, differentiated thyroid carcinoma, carcinoma of the lung, penile carcinoma, adrenocortical carcinoma, endocrine pancreas islet cell carcinoma, colon carcinoma, squamous cell carcinoma, basal cell carcinoma, sweat gland carcinoma, sebaceous gland carcinoma, papillary carcinoma, medullary carcinoma, bronchogenic carcinoma, renal cell carcinoma, anal carcinoma, bile duct carcinoma, choriocarcinoma, embryonal carcinoma, epithelial carcinoma, lymphoma, adult Hodgkin's lymphoma, adult non-Hodgkin's lymphoma, AIDS-related lymphoma, central nervous system lymphoma, cutaneous T-cell lymphoma, T-Cell lymphoma, seminoma, glioblastoma, glioblastoma multiforme, sarcoma, Ewing sarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, soft tissue sarcoma, Kaposi's sarcoma, osteo/malignant fibrous sarcoma, osteosarcoma/malignant fibrous histiocytoma, sarcoidosis sarcoma, uterine sarcoma, lymphangioendotheliosarcoma, leukemia, acute lymphoblastic leukemia, acute lymphocytic leukemia, acute myeloid leukemia, chronic lymphocytic leukemia, chronic myelogenous leukemia, hairy cell leukemia, myelogenous leukemia, myeloid leukemia, myeloblastic leukemia, promyelocytic leukemia, myelomonocytic leukemia, monocytic leukemia, a erythroleukemia, chronic myelocytic leukemia, leukemia myeloma, multiple myeloma, lymphoid malignancies, squamous cell cancer, epithelial squamous cell cancer, squamous cancer of the peritoneum, squamous neck cancer, metastatic squamous neck cancer, metastatic squamous neck cancer, occult metastatic squamous neck cancer, Wilms tumor, astrocytomas, lung cancer, small-cell lung cancer, non-small cell lung cancer, hepatocellular cancer, gastric or stomach cancer, gastrointestinal cancer, gastrointestinal carcinoid tumor, pancreatic cancer, exocrine pancreatic cancer, islet cell pancreatic cancer, cervical cancer, cervical dysplasia, ovarian cancer, ovarian epithelial cancer, ovarian germ cell tumor, ovarian low malignant potential tumor, liver cancer, neuroendocrine tumors, medullary thyroid cancer, parathyroid cancer, breast cancer, colon cancer, rectal cancer, kidney or renal cancer, prostate cancer, vulvar cancer, head-and-neck cancer, AIDS-related malignancies, anal cancer, astrocytoma, cerebellar astrocytoma, cerebral astrocytoma, bile duct cancer, extrahepatic bile duct cancer, bone cancer, fibrous dysplasia of bone, brain tumors, extracranial germ cell tumors, extragonadal germ cell tumor, germ cell tumors, Hodgkin's disease, medulloblastoma, pineal tumors, pinealoma, supratentorial neuroectodermal tumors, ependymoma, epithelial cancer, epithelial dysplasia, mucoepithelial dysplasia, esophageal cancer, esophageal dysplasia, eye cancer, Gaucher's disease, gallbladder cancer, gestational TROPhoblastic tumor, TROPhoblastic tumors, hypergammaglobulinemia, hypopharyngeal cancer, intestinal cancers, intestinal polyps or adenomas, small intestine cancer, large intestine cancer, laryngeal cancer, lip or oral cavity cancer, lymphoproliferative disorders, macroglobulinemia, Waldenstrom's macroglobulinemia, mesothelioma, malignant thymoma, thymoma, metastatic occult plasma cell neoplasm, myelodysplastic syndrome, myeloproliferative disorders, nasal cavity or paranasal sinus cancer, nasopharyngeal cancer, oropharyngeal cancer, paraproteinemias, penile cancer, pheochromocytoma, pituitary tumor, retinoblastoma, salivary gland cancer, Sezary syndrome, skin cancer, testicular cancer, urethral cancer, uterine cancer, vaginal cancer, anhidrotic ectodermal dysplasia, anterofacial dysplasia, asphyxiating thoracic dysplasia, atriodigital dysplasia, bronchopulmonary dysplasia, cerebral dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, faciodigitogenital dysplasia, familial fibrous dysplasia of jaws, familial white folded dysplasia, fibromuscular dysplasia, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, ventriculoradial dysplasia, benign dysproliferative disorders (e.g., benign tumors, fibrocystic conditions, tissue hypertrophy, and), leukoplakia, keratoses, Bowen's disease, Farmer's skin, solar cheilitis, solar keratosis, heavy chain disease, synovioma, craniopharyngioma, emangioblastoma, acoustic neuroma, and meningioma. In some embodiments, the method further comprises administration of one or more additional active agents or supportive therapies for treating the cancer, tumor, pre-neoplastic disorder, hyperproliferative disorder, or dysplastic disorder. In some embodiments, the additional active agent or supportive therapy is an immune checkpoint antagonist, and wherein the immune checkpoint antagonist is selected from a polypeptide, an antibody or antigen-binding fragment thereof, a small molecule, and/or polynucleotide. In some embodiments, the immune checkpoint antagonist is an antibody, or antigen-biding fragment thereof that binds to one or more of: PD-1, PD-L1, CTLA4, BTLA, LAG3, TIM3, LAIR1, B7-DC, HVEM, TIM4, B7-H3, and/or B7-H4. In some embodiments, the antibody is ipilimumab, nivolumab, pembrolizumab, atezolizumab, avelumab, and durvalumab, or an antigen binding fragment thereof. In some embodiments, the additional active agent or supportive therapy is an activin antagonist, and wherein the activin antagonist is selected from a polypeptide, an antibody or antigen-binding fragment thereof, a small molecule, and/or polynucleotide. In some embodiments, the activin antagonist is any of the ActRII polypeptides disclosed herein. In some embodiments, the activin antagonist is any of the ActRII antibodies disclosed herein. In some embodiments, the activin antagonist is any of the activin antibodies disclosed herein. In some embodiments, the additional active agent or supportive therapy is a TGFβ antagonist, and wherein the TGFβ antagonist is selected from a polypeptide, an antibody or antigen-binding fragment thereof, a small molecule, and/or polynucleotide. In some embodiments, the TGFβ antagonist is any of the TβRII polypeptides disclosed herein. In some embodiments, the TGFβ antagonist is any of the TGFβ antibodies disclosed herein. In some embodiments, the TGFβ antagonist is any of the betaglycan polypeptides disclosed herein.

### BRIEF DESCRIPTION OF THE DRAWINGS

FIG. 1 shows the amino acid sequence of native precursor for the B (short) isoform of human TGFβ receptor type II (hTβRII) (NP_003233.4) (SEQ ID NO: 1). Solid underline indicates the processed extracellular domain (ECD) (residues 23-159), and double underline indicates valine that is replaced in the A (long) isoform. Dotted underline denotes leader (residues 1-22).

FIG. 2 shows the amino acid sequence of native precursor for the A (long) isoform of human TβRII (NP_001020018.1) (SEQ ID NO: 2). Solid underline indicates the processed ECD (residues 23-184), and double underline indicates the splice-generated isoleucine substitution. Dotted underline denotes leader (residues 1-22).

FIG. 3 shows a comparison of the linker sequences of five different TβRII constructs.

FIGS. 4A and 4B show in tabular form the binding affinity between TGFβ1 and TGFβ 3 and one of several different TβRII-Fc fusion protein constructs.

FIGS. 5A and 5C graph the results from reporter gene assays testing the affinity of TGFβ1 for one of several different TβRII-Fc fusion protein constructs. FIGS. 5B and 5D graph the results from reporter gene assays testing the affinity of the TGFβ 3 for one of several different TβRII-Fc fusion protein constructs. FIGS. 5E and 5F provide IC₅₀ data from these same experiments in tabular form.

FIG. 6 shows multiple sequence alignment of Fc domains from human IgG isotypes using Clustal 2.1. Hinge regions are indicated by dotted underline. Double underline indicates examples of positions engineered in IgG1 Fc to promote asymmetric chain pairing and the corresponding positions with respect to other isotypes IgG2, IgG3 and IgG4.

FIG. 7 shows an alignment of extracellular domains of human ActRIIA and human ActRIIB with the residues that are deduced herein, based on composite analysis of multiple ActRIIB and ActRIIA crystal structures, to directly contact ligand indicated with boxes.

FIG. 8 shows a multiple sequence alignment of various vertebrate ActRIIB precursor proteins [rat (SEQ ID NO: 101); pig (SEQ ID NO: 102); mouse (SEQ ID NO: 103); human (SEQ ID NO: 104); cow (SEQ ID NO: 108); and xenopus (SEQ ID NO:105)] without their intracellular domains, human ActRIIA precursor protein (SEQ ID NO: 106) without its intracellular domain, and a consensus ActRII precursor protein (SEQ ID NO: 107).

FIG. 9 shows a multiple sequence alignment of various vertebrate ActRIIA proteins without their intracellular domains.

FIG. 10 shows comparative ActRIIB-Fc:TβRII-Fc heterodimer compared to an ActRIIB-Fc:ActRIIB-Fc homodimer and TβRII-Fc:TβRII-Fc homodimer. IC₅₀ data was determined by an A-204 Reporter Gene Assay as described herein. ActRIIB-Fc:TβRII-Fc heterodimer inhibits activin A, activin B, GDF8, GDF11, and BMP10-signaling pathways similarly to the ActRIIB-Fc:ActRIIB-Fc homodimer. However, ActRIIB-Fc:TβRII-Fc heterodimer inhibition of BMP9 signaling pathways is significantly reduced compared to the ActRIIB-Fc:ActRIIB-Fc homodimer. These data demonstrate that ActRIIB-Fc:TβRII-Fc heterodimers are more selective antagonists of activin A, activin B, GDF8, GDF11 and BMP10 compared to corresponding ActRIIB-Fc:ActRIIB-Fc homodimers. In addition the ActRIIB-Fc:TβRII-Fc heterodimer inhibits TGFβ1 and TGFβ 3 signaling pathways similarly to the TβRII-Fc:TβRII-Fc homodimer.

FIG. 11 shows comparative ActRIIA-Fc:TβRII-Fc heterodimer compared to an ActRIIA-Fc:ActRIIA-Fc homodimer and TβRII-Fc:TβRII-Fc homodimer. IC₅₀ data was determined by an A-204 Reporter Gene Assay as described herein. ActRIIA-Fc:TβRII-Fc heterodimer inhibits activin A, activin B, and GDF11 signaling pathways similarly to the ActRIIA-Fc:ActRIIA-Fc homodimer. However, ActRIIA-Fc:TβRII-Fc heterodimer inhibition of BMP10 signaling pathways is significantly reduced compared to the ActRIIA-Fc:ActRIIA-Fc homodimer. These data demonstrate that ActRIIA-Fc:TβRII-Fc heterodimers are more selective antagonists of activin A, activin B, and GDF11 compared to corresponding ActRIIA-Fc:ActRIIA-Fc homodimers. In addition the ActRIIA-Fc:TβRII-Fc heterodimer inhibits TGFβ1 and TGFβ3 signaling pathways similarly to the TβRII-fc:TβRII-fc homodimer.

FIG. 12 shows the amino acid sequence for a truncated, variant ActRIIB (25-131, L79D) domain (SEQ ID NO: 109).

### DETAIL DESCRIPTION OF THE INVENTION

### 1. Overview

The TGFβ superfamily is comprised of over 30 secreted factors including TGFβs, activins, nodals, bone morphogenetic proteins (BMPs), growth and differentiation factors (GDFs), and anti-Mullerian hormone (AMH) [Weiss et al. (2013) Developmental Biology, 2(1): 47-63]. Members of the superfamily, which are found in both vertebrates and invertebrates, are ubiquitously expressed in diverse tissues and function during the earliest stages of development throughout the lifetime of an animal. Indeed, TGFβ superfamily proteins are key mediators of stem cell self-renewal, gastrulation, differentiation, organ morphogenesis, and adult tissue homeostasis. Consistent with this ubiquitous activity, aberrant TGFβ superfamily signaling is associated with a wide range of human pathologies.

Ligands of the TGFβ superfamily share the same dimeric structure in which the central 3½ turn helix of one monomer packs against the concave surface formed by the beta-strands of the other monomer. The majority of TGFβ family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

TGFβ superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massagué (2000) Nat. Rev. Mol. Cell Biol. 1:169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGFβ superfamily ligands. Type I and II receptors form a stable complex after ligand binding, resulting in phosphorylation of type I receptors by type II receptors.

The TGFβ family is divided into two phylogenetic branches based on the type I receptors they bind and the Smad proteins they activate. One is the more recently evolved branch, which includes, e.g., the TGFβs, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMPS, BMP6, BMP7, BMP8a, BMP8b, BMP9, BMP10, GDF1, GDFS, GDF6, and GDF7, which signal through Smads 1, 5, and 8.

TGFβ isoforms are the founding members of the TGFβ superfamily, of which there are 3 known isoforms in mammals designated as TGFβ1, TGFβ2, and TGFβ3. Mature bioactive TGFβ ligands function as homodimers and predominantly signal through the type I receptor ALK5, but have also been found to additionally signal through ALK1 in endothelial cells [Goumans et al. (2003) Mol Cell 12(4): 817-828]. TGFβ1 is the most abundant and ubiquitously expressed isoform. TGFβ1 is known to have an important role in wound healing, and mice expressing a constitutively active TGFβ1 transgene develop fibrosis [Clouthier et al. (1997) J Clin. Invest. 100(11): 2697-2713]. TGFβ1 expression was first described in human glioblastoma cells, and is occurs in neurons and astroglial cells of the embryonic nervous system. TGFβ3 was initially isolated from a human rhabdomyosarcoma cell line and since has been found in lung adenocarcinoma and kidney carcinoma cell lines. TGFβ3 is known to be important for palate and lung morphogenesis [Kubiczkova et al. (2012) Journal of Translational Medicine 10:183].

Activins are members of the TGFβ superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGFβ superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{B}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the β_{B} subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the β_{A} subunit and one or more activities as mediated by the β_{B} subunit.

The BMPs and GDFs together form a family of cysteine-knot cytokines sharing the characteristic fold of the TGFβ superfamily [Rider et al. (2010) Biochem J., 429(1):1-12]. This family includes, for example, BMP2, BMP4, BMP6, BMP7, BMP2a, BMP3, BMP3b (also known as GDF10), BMP4, BMPS, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDFS), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGFβ superfamily ligands.

In part, the data presented herein demonstrates that activin antagonists and TGFβ antagonists can be used alone or in combination to treat cancer. In particular, it was shown that treatment with an ActRIIA polypeptide, an ActRIIB polypeptide, or a pan-specific TGFβ antibody, separately, decreased tumor burden and increased survival time a cancer model. Moreover, it was shown that an activin antagonist in combination with a TGFβ antagonist can be used to synergistically increase antitumor activity compared to the effects observed with either agent alone. While not wishing to be bound by any particular theory, it is believed that such activin and TGFβ antagonist, alone or in combination, may be particularly useful in treating cancer when used in combination with an immune checkpoint antagonist (e.g., an antibody, or antigen-binding fragment thereof, that binds and inhibits one or more of (e.g., PD-1, PD-L1, CTLA4, BTLA, LAG3, TIM3, LAIR1, B7-DC, HVEM, TIM4, B7-H3, and/or B7-H4). Accordingly, the disclosure provides, in part, bi- and tri-functional fusion proteins comprising two or more domains selected from an activin antagonist domain, a TGFβ antagonist domain, and an immune checkpoint antagonist domain. The disclosure further provides, for example, methods of using such bi- and tri-functional fusion proteins to treat cancer, a tumor, a pre-neoplastic disorder, a hyperproliferative disorder, or a dysplastic disorder.

The terms used in this specification generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. The scope or meaning of any use of a term will be apparent from the specific context in which the term is used.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. The term “sequence similarity,” in all its grammatical forms, refers to the degree of identity or correspondence between nucleic acid or amino acid sequences that may or may not share a common evolutionary origin. However, in common usage and in the instant application, the term “homologous,” when modified with an adverb such as “highly,” may refer to sequence similarity and may or may not relate to a common evolutionary origin.

“Percent (%) sequence identity” or “percent (%) identical” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers. As used herein, the term “comprises” also encompasses the use of the narrower terms “consisting” and “consisting essentially of” The term “consisting essentially of” is limited to the specified materials or steps and those that do not materially affect the basic and novel characteristics of the invention(s) disclosed herein.

The term “appreciable affinity” as used herein means binding with a dissociation constant (K_{D}) of less than 50 nM.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

The terms “heteromer” or “heteromultimer” is a complex comprising at least a first polypeptide chain and a second polypeptide chain, wherein the second polypeptide chain differs in amino acid sequence from the first polypeptide chain by at least one amino acid residue. The heteromer can comprise a “heterodimer” formed by the first and second polypeptide chains or can form higher order structures where one or more polypeptide chains in addition to the first and second polypeptide chains are present. Exemplary structures for the heteromultimer include heterodimers, heterotrimers, heterotetramers and further oligomeric structures. Heterodimers are designated herein as X:Y or equivalently as X-Y, where X represents a first polypeptide chain and Y represents a second polypeptide chain Higher-order heteromers and oligomeric structures are designated herein in a corresponding manner. In certain embodiments a heteromultimer is recombinant (e.g., one or more polypeptide components may be a recombinant protein), isolated and/or purified.

### 2. ActRII, TGFβ RII, and Betaglycan Polypeptides

As used herein, the term “TβRII” refers to a family of transforming growth factor beta receptor II (TβRII) proteins from any species and variants derived from such TβRII proteins by mutagenesis or other modification. Reference to TβRII herein is understood to be a reference to any one of the currently identified forms. Members of the TβRII family are generally transmembrane proteins, composed of a ligand-binding extracellular domain comprising a cysteine-rich region, a transmembrane domain, and a cytoplasmic domain with predicted serine/threonine kinase activity. The term “TβRII polypeptide” includes polypeptides comprising any naturally occurring polypeptide of a TβRII family member as well as any variants thereof (including mutants, fragments, fusions, and peptidomimetic forms) that retain a useful activity.

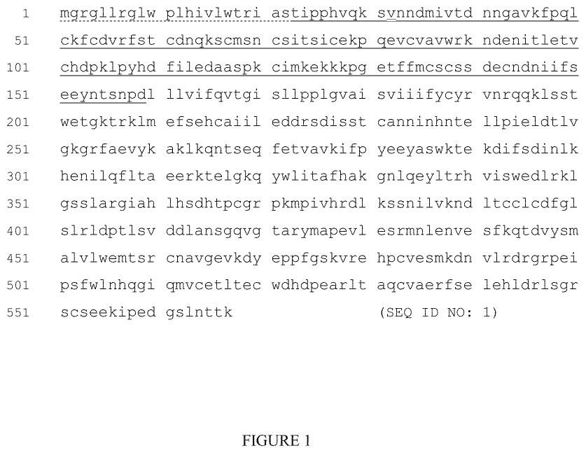

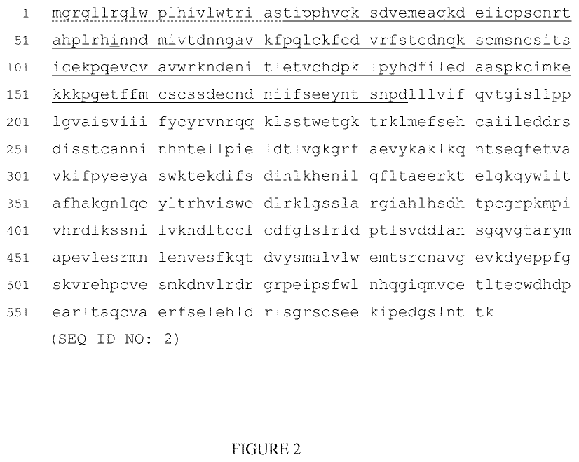

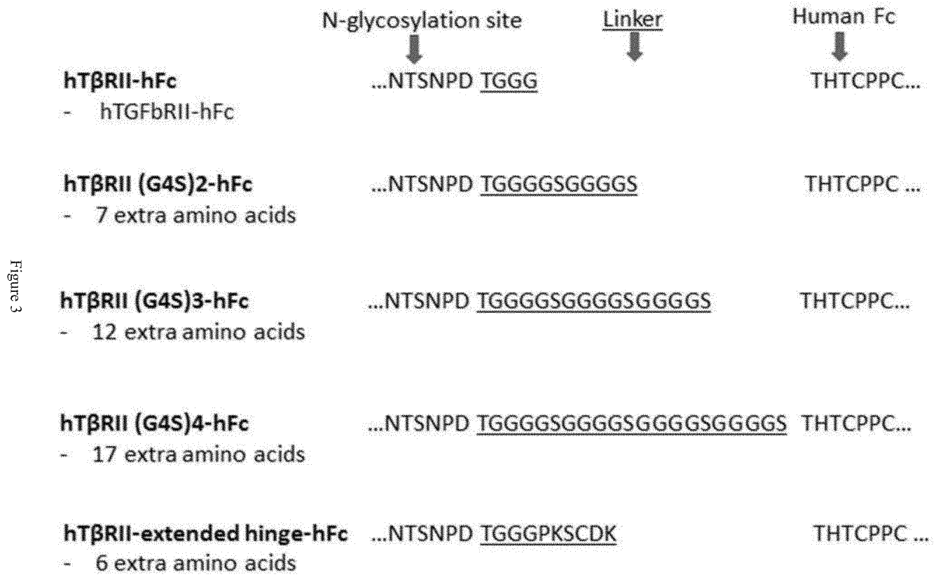

As described above, human TβRII occurs naturally in at least two isoforms—A (long) and B (short)—generated by alternative splicing in the extracellular domain (ECD) (FIGS. 1 and 2 and SEQ ID NOS: 1 and 2). SEQ ID NO: 27, which corresponds to residues 23-159 of SEQ ID NO: 1, depicts the native full-length extracellular domain of the short isoform of TβRII. SEQ ID NO: 18, which corresponds to residues 23-184 of SEQ ID NO: 2, depicts the native full-length extracellular domain of the long isoform of TβRII. Unless noted otherwise, amino acid position numbering with regard to variants based on the TβRII short and long isoforms refers to the corresponding position in the native precursors, SEQ ID NO: 1 and SEQ ID NO: 2, respectively.

In certain embodiments, the disclosure provides variant TβRII polypeptides. A TβRII polypeptide of the disclosure may bind to and inhibit the function of a TGFβ superfamily member, such as but not limited to, TGFβ1 or TGFβ3. TβRII polypeptides may include a polypeptide consisting of, or comprising, an amino acid sequence at least 70% identical, and optionally at least 70%, 75%, 80%, 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 153-159 of SEQ ID NO: 1. TβRII polypeptides may include a polypeptide consisting of, or comprising, an amino acid sequence at least 70% identical, and optionally at least 70%, 75%, 80%, 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 178-184 of SEQ ID NO: 2. In particular embodiments, the TβRII polypeptides comprise an amino acid sequence at least 70% identical, and optionally at least 70%, 75%, 80%, 85%, 90%, 91%, 92%, 93%, 94%, 95%, 96%, 97%, 98%, 99%, or 100% identical to the amino acid sequence of SEQ ID NO: 18. Optionally, a TβRII polypeptide does not include more than 5 consecutive amino acids, or more than 10, 20, 30, 40, 50, 52, 60, 70, 80, 90, 100, 150 or 200 or more consecutive amino acids from a sequence consisting of amino acids 160-567 of SEQ ID NO: 1 or from a sequence consisting of amino acids 185-592 of SEQ ID NO: 2. In some embodiments, the TβRII polypeptide does not include amino acids 160-567 of SEQ ID NO: 1. In some embodiments, the TβRII polypeptide does not include amino acids 1-22 of SEQ ID NO: 1. In some embodiments, the TβRII polypeptide does not include amino acids 1-22 and 160-567 of SEQ ID NO: 1. In some embodiments, the TβRII polypeptide does not include amino acids 185-592 of SEQ ID NO: 2. In some embodiments, the TβRII polypeptide does not include amino acids 1-22 of SEQ ID NO: 2. In some embodiments, the TβRII polypeptide does not include amino acids 1-22 and 185-592 of SEQ ID NO: 2. The unprocessed TβRII polypeptide may either include or exclude any signal sequence, as well as any sequence N-terminal to the signal sequence. As elaborated herein, the N-terminus of the processed TβRII polypeptide may occur at any of amino acids 23-35 of SEQ ID NO: 1 or 23-60 of SEQ ID NO: 2. Examples of processed TβRII polypeptides include, but are not limited to, amino acids 23-159 of SEQ ID NO: 1 (set forth in SEQ ID NO: 27), amino acids 29-159 of SEQ ID NO: 1 (set forth in SEQ ID NO: 28), amino acids 35-159 of SEQ ID NO: 1 (set forth in SEQ ID NO: 29), amino acids 23-153 of SEQ ID NO: 1 (set forth in SEQ ID NO: 30), amino acids 29-153 of SEQ ID NO: 1 (set forth in SEQ ID NO: 31), amino acids 35-153 of SEQ ID NO: 1 (set forth in SEQ ID NO: 32), amino acids 23-184 of SEQ ID NO: 2 (set forth in SEQ ID NO: 18), amino acids 29-184 of SEQ ID NO: 2 (set forth in SEQ ID NO: 33), amino acids 60-184 of SEQ ID NO: 2 (set forth in SEQ ID NO: 29), amino acids 23-178 of SEQ ID NO: 2 (set forth in SEQ ID NO: 34), amino acids 29-178 of SEQ ID NO: 2 (set forth in SEQ ID NO: 35), and amino acids 60-178 of SEQ ID NO: 2 (set forth in SEQ ID NO: 32). It will be understood by one of skill in the art that corresponding variants based on the long isoform of TβRII will include nucleotide sequences encoding the 25-amino acid insertion along with a conservative Val-Ile substitution at the flanking position C-terminal to the insertion. The TβRII polypeptides accordingly may include isolated extracellular portions of TβRII polypeptides, including both the short and the long isoforms, variants thereof (including variants that comprise, for example, no more than 2, 3, 4, 5, 10, 15, 20, 25, 30, or 35 amino acid substitutions in the sequence corresponding to amino acids 23-159 of SEQ ID NO: 1 or amino acids 23-184 of SEQ ID NO: 2), fragments thereof, and fusion proteins comprising any of the foregoing, but in each case preferably any of the foregoing TβRII polypeptides will retain substantial affinity for at least one of, or both of, TGFβ1 or TGFβ3. Generally, a TβRII polypeptide will be designed to be soluble in aqueous solutions at biologically relevant temperatures, pH levels, and osmolarity.

In some embodiments, the variant TβRII polypeptides of the disclosure comprise one or more mutations in the extracellular domain that confer an altered ligand binding profile. A TβRII polypeptide may include one, two, five or more alterations in the amino acid sequence relative to the corresponding portion of a naturally occurring TβRII polypeptide. In some embodiments, the mutation results in a substitution, insertion, or deletion at the position corresponding to position 70 of SEQ ID NO: 1. In some embodiments, the mutation results in a substitution, insertion, or deletion at the position corresponding to position 110 of SEQ ID NO: 1. Examples include, but are not limited to, an N to D substitution or a D to K substitution in the positions corresponding to positions 70 and 110, respectively, of SEQ ID NO: 1. Examples of such variant TβRII polypeptides include, but are not limited to, the sequences set forth in SEQ ID NOs: 36-39. A TβRII polypeptide may comprise a polypeptide or portion thereof that is encoded by any one of SEQ ID NOs: 8, 10, 12, 14, 16, 46 or 47, or silent variants thereof or nucleic acids that hybridize to the complement thereof under stringent hybridization conditions. In particular embodiments, a TβRII polypeptide may comprise a polypeptide or portion thereof that is encoded by any one of SEQ ID NO: 12, or silent variants thereof or nucleic acids that hybridize to the complement thereof under stringent hybridization conditions.

In some embodiments, the variant TβRII polypeptides of the disclosure further comprise an insertion of 36 amino acids (SEQ ID NO: 41) between the pair of glutamate residues (positions 151 and 152 of SEQ ID NO: 1, or positions 176 and 177 of SEQ ID NO: 2) located near the C-terminus of the human TβRII ECD, as occurs naturally in the human TβRII isoform C (Konrad et al., BMC Genomics 8:318, 2007).

It has been demonstrated that TβRII polypeptides can be modified to selectively antagonize TβRII ligands. The N70 residue represents a potential glycosylation site. In some embodiments, the TβRII polypeptides are aglycosylated. In some embodiments, the TβRII polypeptides are aglycosylated or have reduced glycosylation at position Asn157. In some embodiments, the TβRII polypeptides are aglycosylated or have reduced glycosylation at position Asn73.

In certain embodiments, a TβRII polypeptide binds to TGFβ1 and TGFβ3, and the TβRII polypeptide does not show substantial binding to TGFβ2. In certain embodiments, a TβRII polypeptide binds to TGFβ1, TGFβ2, and TGFβ3. Binding may be assessed using purified proteins in solution or in a surface plasmon resonance system, such as a Biacore™ system.

In certain embodiments, a TβRII polypeptide inhibits TGFβ1 and TGFβ 3 cellular signaling, and the TβRII polypeptide has an intermediate or limited inhibitory effect on TGFβ 2 signaling. Inhibitory effect on cell signaling can be assayed by methods known in the art.

Taken together, an active portion of a TβRII polypeptide may comprise amino acid sequences 23-153, 23-154, 23-155, 23-156, 23-157, or 23-158 of SEQ ID NO: 1, as well as variants of these sequences starting at any of amino acids 24-35 of SEQ ID NO: 1. Similarly, an active portion of a TβRII polypeptide may comprise amino acid sequences 23-178, 23-179, 23-180, 23-181, 23-182, or 23-183 of SEQ ID NO: 2, as well as variants of these sequences starting at any of amino acids 24-60 of SEQ ID NO: 2. Exemplary TβRII polypeptides comprise amino acid sequences 29-159, 35-159, 23-153, 29-153 and 35-153 of SEQ ID NO: 1 or amino acid sequences 29-184, 60-184, 23-178, 29-178 and 60-178 of SEQ ID NO: 2. Variants within these ranges are also contemplated, particularly those having at least 70%, 75%, 80%, 85%, 90%, 91%, 92%, 93%, 94%, 95%, 96%, 97%, 98%, or 99% identity to the corresponding portion of SEQ ID NO: 1 or SEQ ID NO: 2. A TβRII polypeptide may be selected that does not include the sequence consisting of amino acids 160-567 of SEQ ID NO: 1 or amino acids 185-592 of SEQ ID NO: 2. In particular embodiments, the TβRII polypeptides comprise an amino acid sequence at least 70% identical, and optionally at least 70%, 75%, 80%, 85%, 90%, 91%, 92%, 93%, 94%, 95%, 96%, 97%, 98%, 99%, or 100% identical to the amino acid sequence of SEQ ID NO: 18.

In some embodiments, the TβRII polypeptides comprise an amino acid sequence that is at least 80%, 85%, 90%, 92%, 94%, 95%, 97%, 99% or 100% identical to the amino acid sequence of any one of SEQ ID NOs: 94-100, or biologically active fragments thereof. In some embodiments, the TβRII polypeptides comprise an amino acid sequence that is at least 80%, 85%, 90%, 92%, 94%, 95%, 97%, 99% or 100% identical to the amino acid sequence of SEQ ID NO: 94, or biologically active fragments thereof. In some embodiments, the TβRII polypeptides comprise an amino acid sequence that is at least 80%, 85%, 90%, 92%, 94%, 95%, 97%, 99% or 100% identical to the amino acid sequence of SEQ ID NO: 98, or biologically active fragments thereof.

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, and WO 2010/151426, which are incorporated herein by reference in their entirety. Numbering of amino acids for all ActRIIB-related polypeptides described herein is based on the numbering of the human ActRIIB precursor protein sequence provided below (SEQ ID NO: 50), unless specifically designated otherwise.

The human ActRIIB precursor protein sequence is as follows:

(SEQ ID NO: 50)

| | |
|---|---|
| 1 | MTAPWVALAL LWGSLCAGSG RGEAETRECI YYNANWELER |
| | T [symbol] QSGLERCE |
| 51 | GEQDKRLHCY ASWR [symbol] SSGTI ELVKKGCWLD DFNCYDRQEC |
| | VATEENPQVY |
| 101 | FCCCEGNFCN ERFTHLPEAG GPEVTYEPPP TAPTLLTVLA |
| | YSLLPIGGLS |
| 151 | LIVLLAFWMY RHRKPPYGHV DIHEDPGPPP PSPLVGLKPL |
| | QLLEIKARGR |
| 201 | FGCVWKAQLM NDFVAVKIFP LQDKQSWQSE REIFSTPGMK |
| | HENLLQFIAA |
| 251 | EKRGSNLEVE LWLITAFHDK GSLTDYLKGN IITWNELCHV |
| | AETMSRGLSY |
| 301 | LHEDVPWCRG EGHKPSIAHR DFKSKNVLLK SDLTAVLADF |
| | GLAVRFEPGK |
| 351 | PPGDTHGQVG TRRYMAPEVL EGAINFQRDA FLRIDMYAMG |
| | LVLWELVSRC |
| 401 | KAADGPVDEY MLPFEEEIGQ HPSLEELQEV VVHKKMRPTI |
| | KDHWLKHPGL |
| 451 | AQLCVTIEEC WDHDAEARLS AGCVEERVSL IRRSVNGTTS |
| | DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

The processed extracellular ActRIIB polypeptide sequence is as follows:

(SEQ ID NO: 51)
GRGEAETRECIYYNANWELERTNQSGLERCEGEQDKRLHCYASWRNSSGTI
ELVKKGCWLDDFNCYDRQECVATEENPQVYFCCCEGNFCNERFTHLPEAGG
PEVTYEPPPTAPT

In some embodiments, the protein may be produced with an “SGR . . . ” sequence at the N-terminus. The C-terminal “tail” of the extracellular domain is indicated by a single underline. The sequence with the “tail” deleted (a Δ15 sequence) is as follows:

(SEQ ID NO: 52)
GRGEAETRECIYYNANWELERTNQSGLERCEGEQDKRLHCYASWRNSSGTI
ELVKKGCWLDDFNCYDRQECVATEENPQVYFCCCEGNFCNERFTHLPEA

A form of ActRIIB with an alanine at position 64 of SEQ ID NO: 1 (A64) is also reported in the literature. See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

```
(SEQ ID NO: 53)
    1 MTAPWVALAL LWGSLCAGSG RGEAETRECI YYNANWELER
TNQSGLERCE
  51 GEQDKRLHCY ASWANSSGTI ELVKKGCWLD DFNCYDRQEC
VATEENPQVY
101 FCCCEGNFCN ERFTHLPEAG GPEVTYEPPP TAPTLLTVLA
YSLLPIGGLS
151 LIVLLAFWMY RHRKPPYGHV DIHEDPGPPP PSPLVGLKPL
QLLEIKARGR
201 FGCVWKAQLM NDFVAVKIFP LQDKQSWQSE REIFSTPGMK
HENLLQFIAA
251 EKRGSNLEVE LWLITAFHDK GSLTDYLKGN IITWNELCHV
AETMSRGLSY
301 LHEDVPWCRG EGHKPSIAHR DFKSKNVLLK SDLTAVLADF
GLAVRFEPGK
351 PPGDTHGQVG TRRYMAPEVL EGAINFQRDA FLRIDMYAMG
LVLWELVSRC
401 KAADGPVDEY MLPFEEEIGQ HPSLEELQEV VVHKKMRPTI
KDHWLKHPGL
451 AQLCVTIEEC WDHDAEARLS AGCVEERVSL IRRSVNGTTS
DCLVSLVTSV
501 TNVDLPPKES SI
```

The signal peptide is indicated by single underline and the extracellular domain is indicated by bold font.

The processed extracellular ActRIIB polypeptide sequence of the alternative A64 form is as follows:

(SEQ ID NO: 54)
GRGEAETRECIYYNANTNELERTNQSGLERCEGEQDKRLHCYASTNANSS
GTIELVKKGCTNLDDFNCYDRQECVATEENPQVYFCCCEGNFCNERFTHL
PEAGGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a Δ15 sequence) is as follows:

(SEQ ID NO: 55)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA

A nucleic acid sequence encoding the human ActRIIB precursor protein is shown below (SEQ ID NO: 56), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

```
(SEQ ID NO: 56)
      1 ATGACGGCGC CCTGGGTGGC CCTCGCCCTC CTCTGGGGAT
CGCTGTGCGC
    51 CGGCTCTGGG CGTGGGGAGG CTGAGACACG GGAGTGCATC
TACTACAACG
  101 CCAACTGGGA GCTGGAGCGC ACCAACCAGA GCGGCCTGGA
GCGCTGCGAA
  151 GGCGAGCAGG ACAAGCGGCT GCACTGCTAC GCCTCCTGGC
GCAACAGCTC
  201 TGGCACCATC GAGCTCGTGA AGAAGGGCTG CTGGCTAGAT
GACTTCAACT
  251 GCTACGATAG GCAGGAGTGT GTGGCCACTG AGGAGAACCC
CCAGGTGTAC
  301 TTCTGCTGCT GTGAAGGCAA CTTCTGCAAC GAACGCTTCA
CTCATTTGCC
  351 AGAGGCTGGG GGCCCGGAAG TCACGTACGA GCCACCCCCG
ACAGCCCCCA
  401 CCCTGCTCAC GGTGCTGGCC TACTCACTGC TGCCCATCGG
GGGCCTTTCC
  451 CTCATCGTCC TGCTGGCCTT TTGGATGTAC CGGCATCGCA
AGCCCCCCTA
  501 CGGTCATGTG GACATCCATG AGGACCCTGG GCCTCCACCA
CCATCCCCTC
  551 TGGTGGGCCT GAAGCCACTG CAGCTGCTGG AGATCAAGGC
TCGGGGGCGC
  601 TTTGGCTGTG TCTGGAAGGC CCAGCTCATG AATGACTTTG
TAGCTGTCAA
  651 GATCTTCCCA CTCCAGGACA AGCAGTCGTG GCAGAGTGAA
CGGGAGATCT
  701 TCAGCACACC TGGCATGAAG CACGAGAACC TGCTACAGTT
CATTGCTGCC
  751 GAGAAGCGAG GCTCCAACCT CGAAGTAGAG CTGTGGCTCA
TCACGGCCTT
  801 CCATGACAAG GGCTCCCTCA CGGATTACCT CAAGGGGAAC
ATCATCACAT
  851 GGAACGAACT GTGTCATGTA GCAGAGACGA TGTCACGAGG
CCTCTCATAC
  901 CTGCATGAGG ATGTGCCCTG GTGCCGTGGC GAGGGCCACA
AGCCGTCTAT
  951 TGCCCACAGG GACTTTAAAA GTAAGAATGT ATTGCTGAAG
AGCGACCTCA
1001 CAGCCGTGCT GGCTGACTTT GGCTTGGCTG TTCGATTTGA
GCCAGGGAAA
1051 CCTCCAGGGG ACACCCACGG ACAGGTAGGC ACGAGACGGT
ACATGGCTCC
1101 TGAGGTGCTC GAGGGAGCCA TCAACTTCCA GAGAGATGCC
TTCCTGCGCA
1151 TTGACATGTA TGCCATGGGG TTGGTGCTGT GGGAGCTTGT
GTCTCGCTGC
1201 AAGGCTGCAG ACGGACCCGT GGATGAGTAC ATGCTGCCCT
TTGAGGAAGA
1251 GATTGGCCAG CACCCTTCGT TGGAGGAGCT GCAGGAGGTG
GTGGTGCACA
1301 AGAAGATGAG GCCCACCATT AAAGATCACT GGTTGAAACA
CCCGGGCCTG
1351 GCCCAGCTTT GTGTGACCAT CGAGGAGTGC TGGGACCATG
ATGCAGAGGC
1401 TCGCTTGTCC GCGGGCTGTG TGGAGGAGCG GGTGTCCCTG
ATTCGGAGGT
1451 CGGTCAACGG CACTACCTCG GACTGTCTCG TTTCCCTGGT
GACCTCTGTC
1501 ACCAATGTGG ACCTGCCCCC TAAAGAGTCA AGCATC
```

A nucleic acid sequence encoding processed extracellular human ActRIIB polypeptide is as follows (SEQ ID NO: 57). The sequence as shown provides an arginine at position 64, and may be modified to provide an alanine instead.

```
(SEQ ID NO: 57)
    1 GGGCGTGGGG AGGCTGAGAC ACGGGAGTGC ATCTACTACA
ACGCCAACTG
  51 GGAGCTGGAG CGCACCAACC AGAGCGGCCT GGAGCGCTGC
GAAGGCGAGC
101 AGGACAAGCG GCTGCACTGC TACGCCTCCT GGCGCAACAG
CTCTGGCACC
151 ATCGAGCTCG TGAAGAAGGG CTGCTGGCTA GATGACTTCA
ACTGCTACGA
201 TAGGCAGGAG TGTGTGGCCA CTGAGGAGAA CCCCCAGGTG
TACTTCTGCT
251 GCTGTGAAGG CAACTTCTGC AACGAACGCT TCACTCATTT
GCCAGAGGCT
301 GGGGGCCCGG AAGTCACGTA CGAGCCACCC CCGACAGCCC
CCACC
```

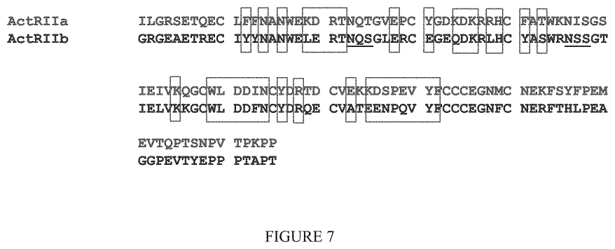

An alignment of the amino acid sequences of human ActRIIB extracellular domain and human ActRIIA extracellular domain are illustrated in FIG. 7. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F101. See FIG. 7. At these positions, it is expected that conservative mutations will be tolerated.

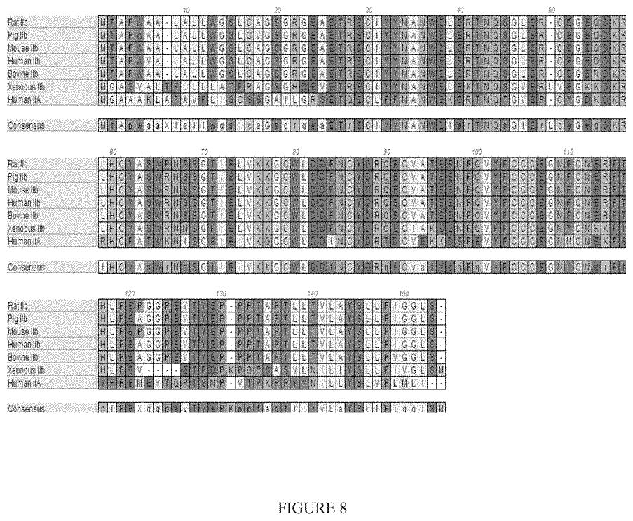

In addition, ActRIIB is well-conserved among vertebrates, with large stretches of the extracellular domain completely conserved. For example, FIG. 8 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant of substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 104) is a valine in Xenopus ActRIIB (SEQ ID NO: 105), and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide ample guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 50 (ActRIIB precursor). Thus, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIB extracellular domains for N-terminal and/or C-terminal truncation include SEQ ID NOs: 51, 52, 54, 55, and 109.

Attisano et al. showed that a deletion of the proline knot at the C-terminus of the extracellular domain of ActRIIB reduced the affinity of the receptor for activin. An ActRIIB-Fc fusion protein containing amino acids 20-119 of precursor SEQ ID NO: 50, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 50) are all expected to be active, but constructs stopping at 134 or 133 may be most active. Similarly, mutations at any of residues 129-134 (with respect to SEQ ID NO: 50) are not expected to alter ligand-binding affinity by large margins. In support of this, it is known in the art that mutations of P129 and P130 (with respect to SEQ ID NO: 50) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO: 50) is poorly conserved and so is readily altered or truncated. ActRIIB polypeptides ending at 128 (with respect to SEQ ID NO: 50) or later should retain ligand-binding activity. ActRIIB polypeptides ending at or between 119 and 127 (e.g., 119, 120, 121, 122, 123, 124, 125, 126, or 127), with respect to SEQ ID NO: 50, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 50) will retain ligand-binding activity. Amino acid 29 represents the initial cysteine. An alanine-to-asparagine mutation at position 24 (with respect to SEQ ID NO: 50) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 50) should retain general ligand-biding activity, and ActRIIB polypeptides beginning at positions 25, 26, 27, 28, and 29 (with respect to SEQ ID NO: 50) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 50. Therefore ActRIIB polypeptides may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 of SEQ ID NO: 50 and ending at a position corresponding to any one amino acids 109-134 of SEQ ID NO: 50. Other examples include polypeptides that begin at a position from 20-29 or 21-29 of SEQ ID NO: 50 and end ata position from 119-134, 119-133, 129-134, or 129-133 of SEQ ID NO: 50. Other examples include constructs that begin at a position from 20-24, 21-24, or 22-25 of SEQ ID NO: 50 and end at a position from 109-134, 119-134 or 129-134 of SEQ ID NO: 50. Variants within these ranges are also contemplated, particularly those having at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 50.

The variations described herein may be combined in various ways. In some embodiments, ActRIIB variants comprise no more than 1, 2, 5, 6, 7, 8, 9, 10 or 15 conservative amino acid changes in the ligand-binding pocket, and zero, one, or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 50). An asparagine-to-alanine alteration at position 65 (N65A) actually improves ligand binding in the A64 background, and is thus expected to have no detrimental effect on ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 50, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 50.

In some embodiments, ActRIIB polypeptides of the disclosure comprise the naturally occurring leucine at the position 79 with respect to SEQ ID NO: 50. In some embodiments, ActRIIB polypeptides of the disclosure comprise an acidic amino acid (e.g., a naturally occurring D or E amino acid residue or an artificial acidic amino acid) at the position 79 with respect to SEQ ID NO: 50. In alternative embodiments, ActRIIB polypeptides of the disclosure do not comprise an acidic amino acid (e.g., a naturally occurring D or E amino acid residue or an artificial acidic amino acid) at the position 79 with respect to SEQ ID NO: 50.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication Nos. WO 2006/012627 and WO 2007/062188, which are incorporated herein by reference in their entirety. Numbering of amino acids for all ActRIIA-related polypeptides described herein is based on the numbering of the human ActRIIA precursor protein sequence provided below (SEQ ID NO: 110), unless specifically designated otherwise.

The human ActRIIA precursor protein sequence is as follows:

```
(SEQ ID NO: 110)
    1 MGAAAKLAFA VFLISCSSGA ILGRSETQEC LFFNANWEKD
RTNQTGVEPC
  51 YGDKDKRRHC FATWKNISGS IEIVKQGCWL DDINCYDRTD
CVEKKDSPEV
101 YFCCCEGNMC NEKFSYFPEM EVTQPTSNPV TPKPPYYNIL
LYSLVPLMLI
151 AGIVICAFWV YRHHKMAYPP VLVPTQDPGP PPPSPLLGLK
PLQLLEVKAR
201 GRFGCVWKAQ LLNEYVAVKI FPIQDKQSWQ NEYEVYSLPG
MKHENILQFI
251 GAEKRGTSVD VDLWLITAFH EKGSLSDFLK ANVVSWNELC
HIAETMARGL
301 AYLHEDIPGL KDGHKPAISH RDIKSKNVLL KNNLTACIAD
FGLALKFEAG
351 KSAGDTHGQV GTRRYMAPEV LEGAINFQRD AFLRIDMYAM
GLVLWELASR
401 CTAADGPVDE YMLPFEEEIG QHPSLEDMQE VVVHKKKRPV
LRDYWQKHAG
451 MAMLCETIEE CWDHDAEARL SAGCVGERIT QMQRLTNIIT
TEDIVTVVTM
501 VTNVDFPPKE SSL
```

The signal peptide is indicated by a single underline; the extracellular domain is indicated in bold font; and the potential, endogenous N-linked glycosylation sites are indicated by a double underline.

A processed extracellular human ActRIIA polypeptide sequence is as follows:

(SEQ ID NO: 111)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by a single underline. The sequence with the “tail” deleted (a Δ15 sequence) is as follows:

(SEQ ID NO: 112)
ILGRSETQECLFFNANWEKDRINQTGVEPCYGDKDKRRHCFATWKNISGS
IEIVKQGCWLDDINCYDRIDCVEKKDSPEVYFCCCEGNMCNEKFSYFFEM

A nucleic acid sequence encoding the human ActRIIA precursor protein is shown below (SEQ ID NO: 113), corresponding to nucleotides 159-1700 of Genbank Reference Sequence NM_001616.4. The signal sequence is underlined.

```
(SEQ ID NO: 113)
      1 ATGGGAGCTG CTGCAAAGTT GGCGTTTGCC GTCTTTCTTA
TCTCCTGTTC
    51 TTCAGGTGCT ATACTTGGTA GATCAGAAAC TCAGGAGTGT
CTTTTCTTTA
  101 ATGCTAATTG GGAAAAAGAC AGAACCAATC AAACTGGTGT
TGAACCGTGT
  151 TATGGTGACA AAGATAAACG GCGGCATTGT TTTGCTACCT
GGAAGAATAT
  201 TTCTGGTTCC ATTGAAATAG TGAAACAAGG TTGTTGGCTG
GATGATATCA
  251 ACTGCTATGA CAGGACTGAT TGTGTAGAAA AAAAAGACAG
CCCTGAAGTA
  301 TATTTTTGTT GCTGTGAGGG CAATATGTGT AATGAAAAGT
TTTCTTATTT
  351 TCCGGAGATG GAAGTCACAC AGCCCACTTC AAATCCAGTT
ACACCTAAGC
  401 CACCCTATTA CAACATCCTG CTCTATTCCT TGGTGCCACT
TATGTTAATT
  451 GCGGGGATTG TCATTTGTGC ATTTTGGGTG TACAGGCATC
ACAAGATGGC
  501 CTACCCTCCT GTACTTGTTC CAACTCAAGA CCCAGGACCA
CCCCCACCTT
  551 CTCCATTACT AGGTTTGAAA CCACTGCAGT TATTAGAAGT
GAAAGCAAGG
  601 GGAAGATTTG GTTGTGTCTG GAAAGCCCAG TTGCTTAACG
AATATGTGGC
  651 TGTCAAAATA TTTCCAATAC AGGACAAACA GTCATGGCAA
AATGAATACG
  701 AAGTCTACAG TTTGCCTGGA ATGAAGCATG AGAACATATT
ACAGTTCATT
  751 GGTGCAGAAA AACGAGGCAC CAGTGTTGAT GTGGATCTTT
GGCTGATCAC
  801 AGCATTTCAT GAAAAGGGTT CACTATCAGA CTTTCTTAAG
GCTAATGTGG
  851 TCTCTTGGAA TGAACTGTGT CATATTGCAG AAACCATGGC
TAGAGGATTG
  901 GCATATTTAC ATGAGGATAT ACCTGGCCTA AAAGATGGCC
ACAAACCTGC
  951 CATATCTCAC AGGGACATCA AAAGTAAAAA TGTGCTGTTG
AAAAACAACC
1001 TGACAGCTTG CATTGCTGAC TTTGGGTTGG CCTTAAAATT
TGAGGCTGGC
1051 AAGTCTGCAG GCGATACCCA TGGACAGGTT GGTACCCGGA
GGTACATGGC
1101 TCCAGAGGTA TTAGAGGGTG CTATAAACTT CCAAAGGGAT
GCATTTTTGA
1151 GGATAGATAT GTATGCCATG GGATTAGTCC TATGGGAACT
GGCTTCTCGC
1201 TGTACTGCTG CAGATGGACC TGTAGATGAA TACATGTTGC
CATTTGAGGA
1251 GGAAATTGGC CAGCATCCAT CTCTTGAAGA CATGCAGGAA
GTTGTTGTGC
1301 ATAAAAAAaA GAGGCCTGTT TTAAGAGATT ATTGGCAGAA
ACATGCTGGA
1351 ATGGCAATGC TCTGTGAAAC CATTGAAGAA TGTTGGGATC
ACGACGCAGA
1401 AGCCAGGTTA TCAGCTGGAT GTGTAGGTGA AAGAATTACC
CAGATGCAGA
1451 GACTAACAAA TATTATTACC ACAGAGGACA TTGTAACAGT
GGTCACAATG
1501 GTGACAAATG TTGACTTTCC TCCCAAAGAA TCTAGTCTA
```

The nucleic acid sequence encoding processed extracellular ActRIIA polypeptide is as follows:

```
(SEQ ID NO: 114)
    1 ATACTTGGTA GATCAGAAAC TCAGGAGTGT CTTTTCTTTA
ATGCTAATTG
  51 GGAAAAAGAC AGAACCAATC AAACTGGTGT TGAACCGTGT
TATGGTGACA
101 AAGATAAACG GCGGCATTGT TTTGCTACCT GGAAGAATAT
TTCTGGTTCC
151 ATTGAAATAG TGAAACAAGG TTGTTGGCTG GATGATATCA
ACTGCTATGA
201 CAGGACTGAT TGTGTAGAAA AAAAAGACAG CCCTGAAGTA
TATTTTTGTT
251 GCTGTGAGGG CAATATGTGT AATGAAAAGT TTTCTTATTT
TCCGGAGATG
301 GAAGTCACAC AGCCCACTTC AAATCCAGTT ACACCTAAGC
CACCC
```

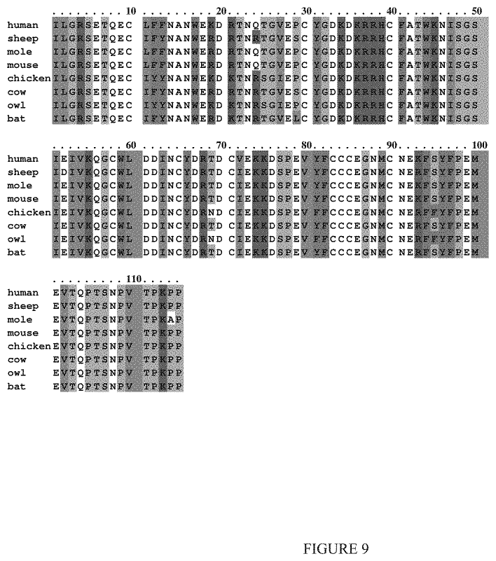

ActRIIA is well-conserved among vertebrates, with large stretches of the extracellular domain completely conserved. For example, FIG. 9 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 9, F13 in the human extracellular domain is Y in Ovis aries (sheep) (SEQ ID NO: 115), Gallus gallus (chicken) (SEQ ID NO: 116), Bos taurus (cow) (SEQ ID NO: 117), Tyto alba (owl) (SEQ ID NO: 118), and Myotis davidii (bat) (SEQ ID NO: 119) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide ample guidance for how to generate ActRIIA variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIA, as demarcated by the outermost of these conserved cysteines, corresponds to positions 30-110 of SEQ ID NO: 110 (ActRIIA precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding Exemplary ActRIIA extracellular domains include SEQ ID NOs: 111 and 112.

Accordingly, a general formula for an active portion (e.g., ligand binding) of ActRIIA is a polypeptide that comprises, consists essentially of, or consists of amino acids 30-110 of SEQ ID NO: 110. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 of SEQ ID NO: 110 and ending at a position corresponding to any one amino acids 110-135 of SEQ ID NO: 110. Other examples include constructs that begin at a position selected from 21-30, 22-30, 23-30, 24-30 of SEQ ID NO: 110, and end at a position selected from 111-135, 112-135, 113-135, 120-135, 130-135, 111-134, 111-133, 111-132, or 111-131 of SEQ ID NO: 110. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 110. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 110. Optionally, ActRIIA polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 110, and comprising no more than 1, 2, 5, 10 or 15 conservative amino acid changes in the ligand-binding pocket.

The term “betaglycan polypeptide” includes polypeptides comprising any naturally occurring betaglycan protein (encoded by TGFBR3 or one of its nonhuman orthologs) as well as any variants thereof (including mutants, fragments, fusions, and peptidomimetic forms) that retain a useful activity.

The human betaglycan isoform A precursor protein sequence (NCBI Ref Seq NP_003234.2) is as follows:

(SEQ ID NO: 120)

| | |
|---|---|
| 1 | MTSHYVIAIF ALMSSCLATA GPEPGALCEL SPVSASHPVQ ALMESFTVLS GCASRGTTGL |
| 61 | PQEVHVLNLR TAGQGPGQLQ REVTLHLNPI SSVHIHHKSV VFLLNSPHPL VWHLKTERLA |
| 121 | TGVSRLFLVS EGSVVQFSSA NFSLTAETEE RNFPHGNEHL LNWARKEYGA VISFTELKIA |
| 181 | RNIYIKVGED QVFPPKCNIG KNFLSLNYLA EYLQPKAAEG CVMSSQPQNE EVHIIELITP |
| 241 | NSNPYSAFQV DITIDIRPSQ EDLEVVKNLI LILKCKKSVN WVIKSFDVKG SLKIIAPNSI |
| 301 | GFGKESERSM TMTKSIRDDI PSTQGNLVKW ALDNGYSPIT SYTMAPVANR FHLRLENNAE |
| 361 | EMGDEEVHTI PPELRILLDP GALPALQNPP IRGGEGQNGG LPFPFPDISR RVWNEEGEDG |
| 421 | LPRPKDPVIP SIQLFPGLRE PEEVQGSVDI ALSVKCDNEK MIVAVEKDSF QASGYSGMDV |
| 481 | TLLDPTCKAK MNGTHFVLES PLNGCGTRPR WSALDGVVYY NSIVIQVPAL GDSSGWPDGY |
| 541 | EDLESGDNGF PGDMDEGDAS LFTRPEIVVF NCSLQQVRNP SSFQEQPHGN ITFNMELYNT |
| 601 | DLFLVPSQGV FSVPENGHVY VEVSVTKAEQ ELGFAIQTCF ISPYSNPDRM SHYTIIENIC |
| 661 | PKDESVKFYS PKRVHFPIPQ ADMDKKRFSF VFKPVFNISL LFLQCELTLC TKMEKHPQKL |
| 721 | PKCVPPDEAC TSLDASIIWA MMQNKKTFIK PLAVIHHEAE SKEKGPSMEE PNPISPPIFH |
| 781 | 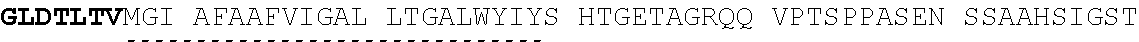 |
| 841 | QSTPCSSSST A |

The signal peptide is indicated by single underline, the extracellular domain is indicated in bold font, and the transmembrane domain is indicated by [symbol] . This isoform differs from betaglycan isoform B by insertion of a single alanine indicated above by double underline.

A processed betaglycan isoform A polypeptide sequence is as follows:

(SEQ ID NO: 121)
GPEPGALCELSPVSASHPVQALMESFTVLSGCASRGTTGLPQEVHVLNLR
TAGQGPGQLQREVTLHLNPISSVHIHHKSVVFLLNSPHPLVWHLKTERLA
TGVSRLFLVSEGSVVQFSSANFSLTAETEERNFPHGNEHLLNWARKEYGA
VTSFTELKIARNIYIKVGEDQVFPPKCNIGKNELSLNYLAEYLQPKAAEG
CVMSSQPQNEEVHIIELITPNSNPYSAFQVDITIDIRPSQEDLEVVKNLI
LILKCKKSVNWVIKSFDVKGSLKIIAPNSIGFGKESERSMTMTKSIRDDI
PSTQGNLVKWALDNGYSPITSYTMAPVANRFHLRLENNAEEMGDEEVHTI
PPELRILLDPGALPALQNPPIRGGEGQNGGLPFPFPDISRRVWNEEGEDG
LPRPKDPVIPSIQLFPGLREPEEVQGSVDIALSVKCDNEKMIVAVEKDSF
QASGYSGMDVTLLDPTCKAKMNGTHEVLESPLNGCGTRPRWSALDGVVYY
NSIVIQVPALGDSSGWPDGYEDLESGDNGFPGDMDEGDASLFTRPEIVVF
NCSLQQVRNPSSFQEQPHGNITFNMELYNTDLFLVPSQGVFSVPENGHVY
VEVSVTKAEQELGFAIQTCFISPYSNPDRMSHYTIIENICPKDESVKFYS
PKRVHFPIPQADMDKKRFSFVFKPVFNTSLLFLQCELTLCTKMEKHPQKL
PKCVPPDEACTSLDASIIWAMMQNKKTFTKPLAVIHHEAESKEKGPSMKE
PNPISPPIFHGLDTLTV

A nucleic acid sequence encoding the unprocessed precursor protein of human betaglycan isoform A is shown below (SEQ ID NO: 122), corresponding to nucleotides 516-3068 of NCBI Reference Sequence NM_003243.4. The signal sequence is indicated by solid underline and the transmembrane region by [symbol] .

(SEQ ID NO: 122)
ATGACTTCCCATTATGTGATTGCCATCTTTGCCCTGATGAGCTCCTG
TTTAGCCACTGCAGGTCCAGAGCCTGGTGCACTGTGTGAACTGTCAC
CTGTCAGTGCCTCCCATCCTGTCCAGGCCTTGATGGAGAGCTTCACT
GTTTTGTCAGGCTGTGCCAGCAGAGGCACAACTGGGCTGCCACAGGA
GGTGCATGTCCTGAATCTCCGCACTGCAGGCCAGGGGCCTGGCCAGC
TACAGAGAGAGGTCACACTTCACCTGAATCCCATCTCCTCAGTCCAC
ATCCACCACAAGTCTGTTGTGTTCCTGCTCAACTCCCCACACCCCCT
GGTGTGGCATCTGAAGACAGAGAGACTTGCCACTGGGGTCTCCAGAC
TGTTTTTGGTGTCTGAGGGTTCTGTGGTCCAGTTTTCATCAGCAAAC
TTCTCCTTGACAGCAGAAACAGAAGAAAGGAACTTCCCCCATGGAAA
TGAACATCTGTTAAATTGGGCCCGAAAAGAGTATGGAGCAGTTACTT
CATTCACCGAACTCAAGATAGCAAGAAACATTTATATTAAAGTGGGG
GAAGATCAAGTGTTCCCTCCAAAGTGCAACATAGGGAAGAATTTTCT
CTCACTCAATTACCTTGCTGAGTACCTTCAACCCAAAGCAGCAGAAG
GGTGTGTGATGTCCAGCCAGCCCCAGAATGAGGAAGTACACATCATC
GAGCTAATCACCCCCAACTCTAACCCCTACAGTGCTTTCCAGGTGGA
TATAACAATTGATATAAGACCTTCTCAAGAGGATCTTGAAGTGGTCA
AAAATCTCATCCTGATCTTGAAGTGCAAAAAGTCTGTCAACTGGGTG
ATCAAATCTTTTGATGTTAAGGGAAGCCTGAAAATTATTGCTCCTAA
CAGTATTGGCTTTGGAAAAGAGAGTGAAAGATCTATGACAATGACCA
AATCAATAAGAGATGACATTCCTTCAACCCAAGGGAATCTGGTGAAG
TGGGCTTTGGACAATGGCTATAGTCCAATAACTTCATACACAATGGC
TCCTGTGGCTAATAGATTTCATCTTCGGCTTGAAAATAATGCAGAGG
AGATGGGAGATGAGGAAGTCCACACTATTCCTCCTGAGCTACGGATC
CTGCTGGACCCTGGTGCCCTGCCTGCCCTGCAGAACCCGCCCATCCG
GGGAGGGGAAGGCCAAAATGGAGGCCTTCCGTTTCCTTTCCCAGATA
TTTCCAGGAGAGTCTGGAATGAAGAGGGAGAAGATGGGCTCCCTCGG
CCAAAGGACCCTGTCATTCCCAGCATACAACTGTTTCCTGGTCTCAG
AGAGCCAGAAGAGGTGCAAGGGAGCGTGGATATTGCCCTGTCTGTCA
AATGTGACAATGAGAAGATGATCGTGGCTGTAGAAAAAGATTCTTTT
CAGGCCAGTGGCTACTCGGGGATGGACGTCACCCTGTTGGATCCTAC
CTGCAAGGCCAAGATGAATGGCACACACTTTGTTTTGGAGTCTCCTC
TGAATGGCTGCGGTACTCGGCCCCGGTGGTCAGCCCTTGATGGTGTG
GTCTACTATAACTCCATTGTGATACAGGTTCCAGCCCTTGGGGACAG
TAGTGGTTGGCCAGATGGTTATGAAGATCTGGAGTCAGGTGATAATG
GATTTCCGGGAGATATGGATGAAGGAGATGCTTCCCTGTTCACCCGA
CCTGAAATCGTGGTGTTTAATTGCAGCCTTCAGCAGGTGAGGAACCC
CAGCAGCTTCCAGGAACAGCCCCACGGAAACATCACCTTCAACATGG
AGCTATACAACACTGACCTCTTTTTGGTGCCCTCCCAGGGCGTCTTC
TCTGTGCCAGAGAATGGACACGTTTATGTTGAGGTATCTGTTACTAA
GGCTGAACAAGAACTGGGATTTGCCATCCAAACGTGCTTTATCTCTC
CATATTCGAACCCTGATAGGATGTCTCATTACACCATTATTGAGAAT
ATTTGTCCTAAAGATGAATCTGTGAAATTCTACAGTCCCAAGAGAGT
GCACTTTCCTATCCCGCAAGCTGACATGGATAAGAAGCGATTCAGCT
TTGTCTTCAAGCCTGTCTTCAACACCTCACTGCTCTTTCTACAGTGT
GAGCTGACGCTGTGTACGAAGATGGAGAAGCACCCCCAGAAGTTGCC
TAAGTGTGTGCCTCCTGACGAAGCCTGCACCTCGCTGGACGCCTCGA
TAATCTGGGCCATGATGCAGAATAAGAAGACGTTCACTAAGCCCCTT
GCTGTGATCCACCATGAAGCAGAATCTAAAGAAAAAGGTCCAAGCAT
GAAGGAACCAAATCCAATTTCTCCACCAATTTTCCATGGTCTGGACA
CCCTAACCGTG

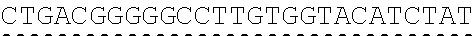

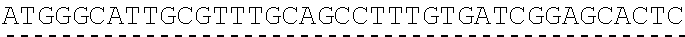

TCTCACACAGGGGAGACAGCAGGAAGGCAGCAAGTCCCCACCTCCCC
GCCAGCCTCGGAAAACAGCAGTGCTGCCCACAGCATCGGCAGCACGC
AGAGCACGCCTTGCTCCAGCAGCAGCACGGCC

A nucleic acid sequence encoding a processed extracellular domain of betaglycan isoform A is shown below (SEQ ID NO: 123):

(SEQ ID NO: 123)
GGTCCAGAGCCTGGTGCACTGTGTGAACTGTCACCTGTCAGTGCCTCCCA
TCCTGTCCAGGCCTTGATGGAGAGCTTCACTGTTTTGTCAGGCTGTGCCA
GCAGAGGCACAACTGGGCTGCCACAGGAGGTGCATGTCCTGAATCTCCGC
ACTGCAGGCCAGGGGCCTGGCCAGCTACAGAGAGAGGTCACACTTCACCT
GAATCCCATCTCCTCAGTCCACATCCACCACAAGTCTGTTGTGTTCCTGC
TCAACTCCCCACACCCCCTGGTGTGGCATCTGAAGACAGAGAGACTTGCC
ACTGGGGTCTCCAGACTGTTTTTGGTGTCTGAGGGTTCTGTGGTCCAGTT
TTCATCAGCAAACTTCTCCTTGACAGCAGAAACAGAAGAAAGGAACTTCC
CCCATGGAAATGAACATCTGTTAAATTGGGCCCGAAAAGAGTATGGAGCA
GTTACTTCATTCACCGAACTCAAGATAGCAAGAAACATTTATATTAAAGT
GGGGGAAGATCAAGTGTTCCCTCCAAAGTGCAACATAGGGAAGAATTTTC
TCTCACTCAATTACCTTGCTGAGTACCTTCAACCCAAAGCAGCAGAAGGG
TGTGTGATGTCCAGCCAGCCCCAGAATGAGGAAGTACACATCATCGAGCT
AATCACCCCCAACTCTAACCCCTACAGTGCTTTCCAGGTGGATATAACAA
TTGATATAAGACCTTCTCAAGAGGATCTTGAAGTGGTCAAAAATCTCATC
CTGATCTTGAAGTGCAAAAAGTCTGTCAACTGGGTGATCAAATCTTTTGA
TGTTAAGGGAAGCCTGAAAATTATTGCTCCTAACAGTATTGGCTTTGGAA
AAGAGAGTGAAAGATCTATGACAATGACCAAATCAATAAGAGATGACATT
CCTTCAACCCAAGGGAATCTGGTGAAGTGGGCTTTGGACAATGGCTATAG
TCCAATAACTTCATACACAATGGCTCCTGTGGCTAATAGATTTCATCTTC
GGCTTGAAAATAATGCAGAGGAGATGGGAGATGAGGAAGTCCACACTATT
CCTCCTGAGCTACGGATCCTGCTGGACCCTGGTGCCCTGCCTGCCCTGCA
GAACCCGCCCATCCGGGGAGGGGAAGGCCAAAATGGAGGCCTTCCGTTTC
CTTTCCCAGATATTTCCAGGAGAGTCTGGAATGAAGAGGGAGAAGATGGG
CTCCCTCGGCCAAAGGACCCTGTCATTCCCAGCATACAACTGTTTCCTGG
TCTCAGAGAGCCAGAAGAGGTGCAAGGGAGCGTGGATATTGCCCTGTCTG
TCAAATGTGACAATGAGAAGATGATCGTGGCTGTAGAAAAAGATTCTTTT
CAGGCCAGTGGCTACTCGGGGATGGACGTCACCCTGTTGGATCCTACCTG
CAAGGCCAAGATGAATGGCACACACTTTGTTTTGGAGTCTCCTCTGAATG
GCTGCGGTACTCGGCCCCGGTGGTCAGCCCTTGATGGTGTGGTCTACTAT
AACTCCATTGTGATACAGGTTCCAGCCCTTGGGGACAGTAGTGGTTGGCC
AGATGGTTATGAAGATCTGGAGTCAGGTGATAATGGATTTCCGGGAGATA
TGGATGAAGGAGATGCTTCCCTGTTCACCCGACCTGAAATCGTGGTGTTT
AATTGCAGCCTTCAGCAGGTGAGGAACCCCAGCAGCTTCCAGGAACAGCC
CCACGGAAACATCACCTTCAACATGGAGCTATACAACACTGACCTCTTTT
TGGTGCCCTCCCAGGGCGTCTTCTCTGTGCCAGAGAATGGACACGTTTAT
GTTGAGGTATCTGTTACTAAGGCTGAACAAGAACTGGGATTTGCCATCCA
AACGTGCTTTATCTCTCCATATTCGAACCCTGATAGGATGTCTCATTACA
CCATTATTGAGAATATTTGTCCTAAAGATGAATCTGTGAAATTCTACAGT
CCCAAGAGAGTGCACTTTCCTATCCCGCAAGCTGACATGGATAAGAAGCG
ATTCAGCTTTGTCTTCAAGCCTGTCTTCAACACCTCACTGCTCTTTCTAC
AGTGTGAGCTGACGCTGTGTACGAAGATGGAGAAGCACCCCCAGAAGTTG
CCTAAGTGTGTGCCTCCTGACGAAGCCTGCACCTCGCTGGACGCCTCGAT
AATCTGGGCCATGATGCAGAATAAGAAGACGTTCACTAAGCCCCTTGCTG
TGATCCACCATGAAGCAGAATCTAAAGAAAAAGGTCCAAGCATGAAGGAA
CCAAATCCAATTTCTCCACCAATTTTCCATGGTCTGGACACCCTAACCGT
G

A human betaglycan isoform B precursor protein sequence (NCBI Ref Seq NP_001182612.1) is as follows:

(SEQ ID NO: 124)

| | |
|---|---|
| 1 | MTSHYVIAIF ALMSSCLATA GPEPGALCEL SPVSASHPVQ ALMESFTVLS GCASRGTTGL |
| 61 | PQEVHVLNLR TAGQGPGQLQ REVTLHLNPI SSVHIHHKSV VFLLNSPHPL VWHLKTERLA |
| 121 | TGVSRLFLVS EGSVVQFSSA NFSLTAETEE RNFPHGNEHL LNWARKEYGA VISFTELKIA |
| 181 | RNIYIKVGED QVFPPKCNIG KNFLSLNYLA EYLQPKAAEG CVMSSQPQNE EVHIIELITP |
| 241 | NSNPYSAFQV DITIDIRPSQ EDLEVVKNLI LILKCKKSVN WVIKSFDVKG SLKIIAPNSI |
| 301 | GFGKESERSM TMTKSIRDDI PSTQGNLVKW ALDNGYSPIT SYTMAPVANR FHLRLENNEE |
| 361 | MGDEEVHTIP PELRILLDPG ALPALQNPPI RGGEGQNGGL PFPFPDISRR VWNEEGEDGL |
| 421 | PRPKDPVIPS IQLFPGLREP EEVQGSVDIA LSVKCDNEKM IVAVEKDSFQ ASGYSGMDVT |
| 481 | LLDPTCKAKM NGTHFVLESP LNGCGTRPRW SALDGVVYYN SIVIQVPALG DSSGWPDGYE |
| 541 | DLESGDNGFP GDMDEGDASL FTRPEIVVFN CSLQQVRNPS SFQEQPHGNI TFNMELYNTD |
| 601 | LFLVPSQGVF SVPENGHVYV EVSVTKAEQE LGFAIQTCFI SPYSNPDRMS HYTIIENICP |
| 661 | KDESVKFYSP KRVHFPIPQA DMDKKRFSFV FKPVFNTSLL FLQCELTLCT KMEKHPQKLP |
| 721 | KCVPPDEACT SLDASIIWAM MQNKKTFTKP LAVIHHEAES KEKGPSMKEP NPISPPIFHG |
| 781 | 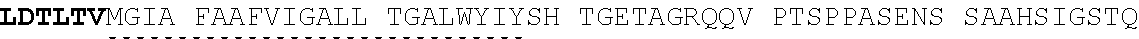 |
| 841 | STPCSSSSTA |

The signal peptide is indicated by single underline, the extracellular domain is indicated in bold font, and the transmembrane domain is indicated by [symbol] .

A processed betaglycan isoform B polypeptide sequence is as follows:

(SEQ ID NO: 125)
GPEPGALCELSPVSASHPVQALMESFTVLSGCASRGTTGLPQEVHVLNLR
TAGQGPGQLQREVTLHLNPISSVHIHHKSVVFLLNSPHPLVWHLKTERLA
TGVSRLFLVSEGSVVQFSSANFSLTAETEERNFPHGNEHLLNWARKEYGA
VTSFTELKIARNIYIKVGEDQVFPPKCNIGKNFLSLNYLAEYLQPKAAEG
CVMSSQPQNEEVHIIELITPNSNPYSAFQVDITIDIRPSQEDLEVVKNLI
LILKCKKSVNWVIKSFDVKGSLKIIAPNSIGFGKESERSMTMTKSIRDDI
PSTQGNLVKWALDNGYSPITSYTMAPVANRFHLRLENNEEMGDEEVHTIP
PELRILLDPGALPALQNPPIRGGEGQNGGLPFPFPDISRRVWNEEGEDGL
PRPKDPVIPSIQLFPGLREPEEVQGSVDIALSVKCDNEKMIVAVEKDSFQ
ASGYSGMDVTLLDPTCKAKMNGTHFVLESPLNGCGTRPRWSALDGVVYYN
SIVIQVPALGDSSGWPDGYEDLESGDNGFPGDMDEGDASLFTRPEIVVFN
CSLQQVRNPSSFQEQPHGNITFNMELYNTDLFLVPSQGVFSVPENGHVYV
EVSVTKAEQELGFAIQTCFISPYSNPDRMSHYTIIENICPKDESVKFYSP
KRVHFPIPQADMDKKRFSFVFKPVFNTSLLFLQCELTLCTKMEKHPQKLP
KCVPPDEACTSLDASIIWAMMQNKKTFTKPLAVIHHEAESKEKGPSMKEP
NPISPPIFHGLDTLTV

A nucleic acid sequence encoding the unprocessed precursor protein of human betaglycan isoform B is shown below (SEQ ID NO: 126), corresponding to nucleotides 516-3065 of NCBI Reference Sequence NM_001195683.1. The signal sequence is indicated by solid underline and the transmembrane region by [symbol] .

(SEQ ID NO: 126)
ATGACTTCCCATTATGTGATTGCCATCTTTGCCCTGATGAGCTCCTG
TTTAGCCACTGCAGGTCCAGAGCCTGGTGCACTGTGTGAACTGTCAC
CTGTCAGTGCCTCCCATCCTGTCCAGGCCTTGATGGAGAGCTTCACT
GTTTTGTCAGGCTGTGCCAGCAGAGGCACAACTGGGCTGCCACAGGA
GGTGCATGTCCTGAATCTCCGCACTGCAGGCCAGGGGCCTGGCCAGC
TACAGAGAGAGGTCACACTTCACCTGAATCCCATCTCCTCAGTCCAC
ATCCACCACAAGTCTGTTGTGTTCCTGCTCAACTCCCCACACCCCCT
GGTGTGGCATCTGAAGACAGAGAGACTTGCCACTGGGGTCTCCAGAC
TGTTTTTGGTGTCTGAGGGTTCTGTGGTCCAGTTTTCATCAGCAAAC
TTCTCCTTGACAGCAGAAACAGAAGAAAGGAACTTCCCCCATGGAAA
TGAACATCTGTTAAATTGGGCCCGAAAAGAGTATGGAGCAGTTACTT
CATTCACCGAACTCAAGATAGCAAGAAACATTTATATTAAAGTGGGG
GAAGATCAAGTGTTCCCTCCAAAGTGCAACATAGGGAAGAATTTTCT
CTCACTCAATTACCTTGCTGAGTACCTTCAACCCAAAGCAGCAGAAG
GGTGTGTGATGTCCAGCCAGCCCCAGAATGAGGAAGTACACATCATC
GAGCTAATCACCCCCAACTCTAACCCCTACAGTGCTTTCCAGGTGGA
TATAACAATTGATATAAGACCTTCTCAAGAGGATCTTGAAGTGGTCA
AAAATCTCATCCTGATCTTGAAGTGCAAAAAGTCTGTCAACTGGGTG
ATCAAATCTTTTGATGTTAAGGGAAGCCTGAAAATTATTGCTCCTAA
CAGTATTGGCTTTGGAAAAGAGAGTGAAAGATCTATGACAATGACCA
AATCAATAAGAGATGACATTCCTTCAACCCAAGGGAATCTGGTGAAG
TGGGCTTTGGACAATGGCTATAGTCCAATAACTTCATACACAATGGC
TCCTGTGGCTAATAGATTTCATCTTCGGCTTGAAAATAATGAGGAGA
TGGGAGATGAGGAAGTCCACACTATTCCTCCTGAGCTACGGATCCTG
CTGGACCCTGGTGCCCTGCCTGCCCTGCAGAACCCGCCCATCCGGGG
AGGGGAAGGCCAAAATGGAGGCCTTCCGTTTCCTTTCCCAGATATTT
CCAGGAGAGTCTGGAATGAAGAGGGAGAAGATGGGCTCCCTCGGCCA
AAGGACCCTGTCATTCCCAGCATACAACTGTTTCCTGGTCTCAGAGA
GCCAGAAGAGGTGCAAGGGAGCGTGGATATTGCCCTGTCTGTCAAAT
GTGACAATGAGAAGATGATCGTGGCTGTAGAAAAAGATTCTTTTCAG
GCCAGTGGCTACTCGGGGATGGACGTCACCCTGTTGGATCCTACCTG
CAAGGCCAAGATGAATGGCACACACTTTGTTTTGGAGTCTCCTCTGA
ATGGCTGCGGTACTCGGCCCCGGTGGTCAGCCCTTGATGGTGTGGTC
TACTATAACTCCATTGTGATACAGGTTCCAGCCCTTGGGGACAGTAG
TGGTTGGCCAGATGGTTATGAAGATCTGGAGTCAGGTGATAATGGAT
TTCCGGGAGATATGGATGAAGGAGATGCTTCCCTGTTCACCCGACCT
GAAATCGTGGTGTTTAATTGCAGCCTTCAGCAGGTGAGGAACCCCAG
CAGCTTCCAGGAACAGCCCCACGGAAACATCACCTTCAACATGGAGC
TATACAACACTGACCTCTTTTTGGTGCCCTCCCAGGGCGTCTTCTCT
GTGCCAGAGAATGGACACGTTTATGTTGAGGTATCTGTTACTAAGGC
TGAACAAGAACTGGGATTTGCCATCCAAACGTGCTTTATCTCTCCAT
ATTCGAACCCTGATAGGATGTCTCATTACACCATTATTGAGAATATT
TGTCCTAAAGATGAATCTGTGAAATTCTACAGTCCCAAGAGAGTGCA
CTTTCCTATCCCGCAAGCTGACATGGATAAGAAGCGATTCAGCTTTG
TCTTCAAGCCTGTCTTCAACACCTCACTGCTCTTTCTACAGTGTGAG
CTGACGCTGTGTACGAAGATGGAGAAGCACCCCCAGAAGTTGCCTAA
GTGTGTGCCTCCTGACGAAGCCTGCACCTCGCTGGACGCCTCGATAA
TCTGGGCCATGATGCAGAATAAGAAGACGTTCACTAAGCCCCTTGCT
GTGATCCACCATGAAGCAGAATCTAAAGAAAAAGGTCCAAGCATGAA
GGAACCAAATCCAATTTCTCCACCAATTTTCCATGGTCTGGACACCC

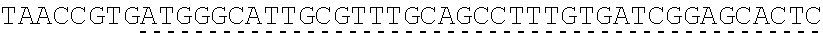

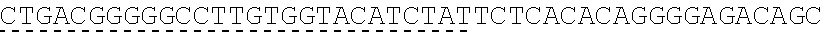

AGGAAGGCAGCAAGTCCCCACCTCCCCGCCAGCCTCGGAAAACAGCA
GTGCTGCCCACAGCATCGGCAGCACGCAGAGCACGCCTTGCTCCAGC
AGCAGCACGGCC

A nucleic acid sequence encoding a processed extracellular domain of betaglycan isoform B is shown below (SEQ ID NO: 127):

(SEQ ID NO: 127)
GGTCCAGAGCCTGGTGCACTGTGTGAACTGTCACCTGTCAGTGCCTCCCA
TCCTGTCCAGGCCTTGATGGAGAGCTTCACTGTTTTGTCAGGCTGTGCCA
GCAGAGGCACAACTGGGCTGCCACAGGAGGTGCATGTCCTGAATCTCCGC
ACTGCAGGCCAGGGGCCTGGCCAGCTACAGAGAGAGGTCACACTTCACCT
GAATCCCATCTCCTCAGTCCACATCCACCACAAGTCTGTTGTGTTCCTGC
TCAACTCCCCACACCCCCTGGTGTGGCATCTGAAGACAGAGAGACTTGCC
ACTGGGGTCTCCAGACTGTTTTTGGTGTCTGAGGGTTCTGTGGTCCAGTT
TTCATCAGCAAACTTCTCCTTGACAGCAGAAACAGAAGAAAGGAACTTCC
CCCATGGAAATGAACATCTGTTAAATTGGGCCCGAAAAGAGTATGGAGCA
GTTACTTCATTCACCGAACTCAAGATAGCAAGAAACATTTATATTAAAGT
GGGGGAAGATCAAGTGTTCCCTCCAAAGTGCAACATAGGGAAGAATTTTC
TCTCACTCAATTACCTTGCTGAGTACCTTCAACCCAAAGCAGCAGAAGGG
TGTGTGATGTCCAGCCAGCCCCAGAATGAGGAAGTACACATCATCGAGCT
AATCACCCCCAACTCTAACCCCTACAGTGCTTTCCAGGTGGATATAACAA
TTGATATAAGACCTTCTCAAGAGGATCTTGAAGTGGTCAAAAATCTCATC
CTGATCTTGAAGTGCAAAAAGTCTGTCAACTGGGTGATCAAATCTTTTGA
TGTTAAGGGAAGCCTGAAAATTATTGCTCCTAACAGTATTGGCTTTGGAA
AAGAGAGTGAAAGATCTATGACAATGACCAAATCAATAAGAGATGACATT
CCTTCAACCCAAGGGAATCTGGTGAAGTGGGCTTTGGACAATGGCTATAG
TCCAATAACTTCATACACAATGGCTCCTGTGGCTAATAGATTTCATCTTC
GGCTTGAAAATAATGAGGAGATGGGAGATGAGGAAGTCCACACTATTCCT
CCTGAGCTACGGATCCTGCTGGACCCTGGTGCCCTGCCTGCCCTGCAGAA
CCCGCCCATCCGGGGAGGGGAAGGCCAAAATGGAGGCCTTCCGTTTCCTT
TCCCAGATATTTCCAGGAGAGTCTGGAATGAAGAGGGAGAAGATGGGCTC
CCTCGGCCAAAGGACCCTGTCATTCCCAGCATACAACTGTTTCCTGGTCT
CAGAGAGCCAGAAGAGGTGCAAGGGAGCGTGGATATTGCCCTGTCTGTCA
AATGTGACAATGAGAAGATGATCGTGGCTGTAGAAAAAGATTCTTTTCAG
GCCAGTGGCTACTCGGGGATGGACGTCACCCTGTTGGATCCTACCTGCAA
GGCCAAGATGAATGGCACACACTTTGTTTTGGAGTCTCCTCTGAATGGCT
GCGGTACTCGGCCCCGGTGGTCAGCCCTTGATGGTGTGGTCTACTATAAC
TCCATTGTGATACAGGTTCCAGCCCTTGGGGACAGTAGTGGTTGGCCAGA
TGGTTATGAAGATCTGGAGTCAGGTGATAATGGATTTCCGGGAGATATGG
ATGAAGGAGATGCTTCCCTGTTCACCCGACCTGAAATCGTGGTGTTTAAT
TGCAGCCTTCAGCAGGTGAGGAACCCCAGCAGCTTCCAGGAACAGCCCCA
CGGAAACATCACCTTCAACATGGAGCTATACAACACTGACCTCTTTTTGG
TGCCCTCCCAGGGCGTCTTCTCTGTGCCAGAGAATGGACACGTTTATGTT
GAGGTATCTGTTACTAAGGCTGAACAAGAACTGGGATTTGCCATCCAAAC
GTGCTTTATCTCTCCATATTCGAACCCTGATAGGATGTCTCATTACACCA
TTATTGAGAATATTTGTCCTAAAGATGAATCTGTGAAATTCTACAGTCCC
AAGAGAGTGCACTTTCCTATCCCGCAAGCTGACATGGATAAGAAGCGATT
CAGCTTTGTCTTCAAGCCTGTCTTCAACACCTCACTGCTCTTTCTACAGT
GTGAGCTGACGCTGTGTACGAAGATGGAGAAGCACCCCCAGAAGTTGCCT
AAGTGTGTGCCTCCTGACGAAGCCTGCACCTCGCTGGACGCCTCGATAAT
CTGGGCCATGATGCAGAATAAGAAGACGTTCACTAAGCCCCTTGCTGTGA
TCCACCATGAAGCAGAATCTAAAGAAAAAGGTCCAAGCATGAAGGAACCA
AATCCAATTTCTCCACCAATTTTCCATGGTCTGGACACCCTAACCGTG

In certain embodiments, the disclosure relates to bi- or tri-functional fusion proteins that comprise at least one betaglycan polypeptide, which includes fragments, functional variants, and modified forms thereof. Preferably, betaglycan polypeptides for use in accordance with inventions of the disclosure are soluble (e.g., an extracellular, ligand-binding domain of betaglycan). In other preferred embodiments, betaglycan polypeptides for use in accordance with the inventions of the disclosure bind to and inhibit activity (e.g., Smad signaling) of one or more TGFβ isoforms (TGFβ1, TGFβ2, and/or TGFβ3). In some embodiments, bi- or tri-functional fusion proteins of the disclosure comprise of at least one betaglycan polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 121 or 125. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 of SEQ ID NO: 121, and ends at any one of amino acids 381-787 of SEQ ID NO: 121. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-381 of SEQ ID NO: 121. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-787 of SEQ ID NO: 121. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-381 of SEQ ID NO: 121. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-787 of SEQ ID NO: 121. In some embodiments, bi- or tri-functional fusion proteins of the disclosure comprise of at least one betaglycan polypeptide that is at least 70%, 75%, 80%, 85%, 90%, 91%, 92%, 93%, 94%, 95%, 96%, 97%, 98%, 99%, or 100% identical to amino acids of 21-781 of SEQ ID NO: 121. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-781 of SEQ ID NO: 121. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 of SEQ ID NO: 125, and ends at any one of amino acids 380-786 of SEQ ID NO: 125. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-380 of SEQ ID NO: 125. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-786 of SEQ ID NO: 125. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-380 of SEQ ID NO: 125. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-786 of SEQ ID NO: 125. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-780 of SEQ ID NO: 125. In some embodiments, bi- or tri-functional fusion proteins of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-780 of SEQ ID NO: 125.

As described above, the disclosure provides TβRII, ActRIIB, ActRIIA, or betaglycan polypeptides sharing a specified degree of sequence identity or similarity to a naturally occurring TβRII, ActRIIB, ActRIIA, or betaglycan polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In one embodiment, the percent identity between two amino acid sequences is determined using the Needleman and Wunsch (J Mol. Biol. (48):444-453 (1970)) algorithm which has been incorporated into the GAP program in the GCG software package (available at http://www.gcg.com). In a specific embodiment, the following parameters are used in the GAP program: either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux, J., et al., Nucleic Acids Res. 12(1):387 (1984)) (available at http://www.gcg.com). Exemplary parameters include using a NWSgapdna.CMP matrix and a gap weight of 40, 50, 60, 70, or 80 and a length weight of 1, 2, 3, 4, 5, or 6. Unless otherwise specified, percent identity between two amino acid sequences is to be determined using the GAP program using a Blosum 62 matrix, a GAP weight of 10 and a length weight of 3, and if such algorithm cannot compute the desired percent identity, a suitable alternative disclosed herein should be selected.

In another embodiment, the percent identity between two amino acid sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

Another embodiment for determining the best overall alignment between two amino acid sequences can be determined using the FASTDB computer program based on the algorithm of Brutlag et al. (Comp. App. Biosci., 6:237-245 (1990)). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty=0.05.

Polypeptides of the disclosure (e.g., TβRII, ActRIIA, ActRIIB, or betaglycan polypeptides) may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native signal sequence (e.g., native TβRII, ActRIIA, ActRIIB, or betaglycan signal sequence) may be used to effect extrusion from the cell. Possible leader sequences include native leaders, tissue plasminogen activator (TPA) and honeybee mellitin (SEQ ID NOs. 22-24, respectively). Examples of fusion proteins incorporating a TPA leader sequence include SEQ ID NOs: 9, 11, 13, 15, 17, 82, 85, 88, 91, and 110. Processing of signal peptides may vary depending on the leader sequence chosen, the cell type used and culture conditions, among other variables, and therefore actual N-terminal start sites for processed polypeptides may shift by 1, 2, 3, 4 or 5 amino acids in either the N-terminal or C-terminal direction. It will be understood by one of skill in the art that corresponding variants based on the long isoform of TβRII will include the 25-amino acid insertion along with a conservative Val-Ile substitution at the flanking position C-terminal to the insertion.

In certain embodiments, the present disclosure contemplates specific mutations of the polypeptides (e.g., TβRII, ActRIIA, ActRIIB, betaglycan polypeptides) so as to alter the glycosylation of the polypeptide. Such mutations may be selected to introduce or eliminate one or more glycosylation sites, such as O-linked or N-linked glycosylation sites. Asparagine-linked glycosylation recognition sites generally comprise a tripeptide sequence, asparagine-X-threonine (or asparagine-X-serine) (where “X” is any amino acid) which is specifically recognized by appropriate cellular glycosylation enzymes. The alteration may also be made by the addition of, or substitution by, one or more serine or threonine residues to the sequence of the wild-type polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on a polypeptide may be accomplished chemically and/or enzymatically. Chemical deglycosylation may involve, for example, exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on polypeptides can be achieved by the use of a variety of endo- and exo-glycosidases as described by Thotakura et al. (1987) Meth. Enzymol. 138:350. The sequence of a polypeptide may be adjusted, as appropriate, depending on the type of expression system used, as mammalian, yeast, insect and plant cells may all introduce differing glycosylation patterns that can be affected by the amino acid sequence of the peptide. In general, polypeptides (e.g., TβRII, ActRIIA, ActRIIB, or betaglycan polypeptides) for use in humans will be expressed in a mammalian cell line that provides proper glycosylation, such as HEK293 or CHO cell lines, although other mammalian expression cell lines, yeast cell lines with engineered glycosylation enzymes, and insect cells are expected to be useful as well.

This disclosure further contemplates a method of generating mutants, particularly sets of combinatorial mutants of a polypeptide (e.g., TβRII, ActRIIA ActRIIB, or betaglycan polypeptides), as well as truncation mutants; pools of combinatorial mutants are especially useful for identifying functional variant sequences. The purpose of screening such combinatorial libraries may be to generate, for example, polypeptide variants which can act as either agonists or antagonist, or alternatively, which possess novel activities all together. A variety of screening assays are provided below, and such assays may be used to evaluate variants. For example, a bi- or tri-functional fusion protein comprising an ActRIIB, ActRIIA, betaglycan, and/or TβRII polypeptide variant may be screened for ability to bind to an AcRIIB, ActRIIA, betaglycan, or TβRII ligand, to prevent binding of an ActRIIB, ActRIIA, betaglycan, or TβRII ligand to an ActRIIB, ActRIIA, betaglycan or TβRII polypeptide or to interfere with signaling caused by an ActRIIB, ActRIIA, betaglycan or TβRII ligand.

Combinatorially-derived variants can be generated which have a selective or generally increased potency relative to a polypeptide (e.g., TβRII, ActRIIA, ActRIIB, or betaglycan polypeptides) comprising an extracellular domain of a naturally occurring polypeptide. Likewise, mutagenesis can give rise to variants which have serum half-lives dramatically different than the corresponding wild-type polypeptide. For example, the altered protein can be rendered either more stable or less stable to proteolytic degradation or other processes which result in destruction of, or otherwise elimination or inactivation of, a native TβRII polypeptide. Such variants, and the genes which encode them, can be utilized to alter TβRII polypeptide levels by modulating the half-life of the TβRII polypeptides. For instance, a short half-life can give rise to more transient biological effects and can allow tighter control of recombinant polypeptide levels within the patient. In an Fc fusion protein, mutations may be made in the linker (if any) and/or the Fc portion to alter the half-life of the protein.

A combinatorial library may be produced by way of a degenerate library of genes encoding a library of polypeptides which each include at least a portion of potential polypeptide (e.g., TβRII, ActRIIA, ActRIIB, or betaglycan polypeptides) sequences. For instance, a mixture of synthetic oligonucleotides can be enzymatically ligated into gene sequences such that the degenerate set of potential polypeptide nucleotide sequences are expressible as individual polypeptides, or alternatively, as a set of larger fusion proteins (e.g., for phage display).

There are many ways by which the library of potential polypeptide (e.g., TβRII, ActRIIA, ActRIIB, or betaglycan polypeptide) variants can be generated from a degenerate oligonucleotide sequence. Chemical synthesis of a degenerate gene sequence can be carried out in an automatic DNA synthesizer, and the synthetic genes then be ligated into an appropriate vector for expression. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, polypeptide (e.g., TβRII, ActRIIA, ActRIIB, or betaglycan polypeptide) variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of polypeptides (e.g., TβRII, ActRIIA, ActRIIB, or betaglycan polypeptides). The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include ligand binding assays and ligand-mediated cell signaling assays.

In certain embodiments, the polypeptides (e.g., TβRII, ActRIIA, ActRIIB, betaglycan polypeptides) of the disclosure may further comprise post-translational modifications in addition to any that are naturally present in the native polypeptides. Such modifications include, but are not limited to, acetylation, carboxylation, glycosylation, phosphorylation, lipidation, pegylation (polyethylene glycol) and acylation. As a result, the modified polypeptides may contain non-amino acid elements, such as polyethylene glycols, lipids, mono- or poly-saccharides, and phosphates. Effects of such non-amino acid elements on the functionality of a polypeptide may be tested as described herein for other polypeptide variants. When a polypeptide is produced in cells by cleaving a nascent form of the polypeptide, post-translational processing may also be important for correct folding and/or function of the protein. Different cells (such as CHO, HeLa, MDCK, 293, WI38, NIH-3T3 or HEK-293) have specific cellular machinery and characteristic mechanisms for such post-translational activities and may be chosen to ensure the correct modification and processing of the polypeptides.

In certain aspects, the disclosure provides for fusion proteins, and in some embodiments, a first portion is connected to a heterologous portion (e.g., Fc portion) by means of a linker. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In some preferred embodiments, the linker comprises the amino acid sequence GlyGlyGlyGlySer (GGGGS) (SEQ ID NO: 19), or repetitions thereof (GGGGS)n, where n≥2. In particular embodiments n≥3, or n=3-10. The application teaches the surprising finding that proteins comprising a TβRII portion and a heterologous portion fused together by means of a (GGGGS)₄ linker were associated with a stronger affinity for TGFβ1 and TGFβ 3 as compared to a TβRII fusion protein where n<4. As such, in preferred embodiments, n≥4, or n=4-10. The application also teaches that proteins comprising (GGGGS)ₙ linkers in which n>4 had similar inhibitory properties as proteins having the (GGGGS)₄ linker. As such, in some embodiments, n is not greater than 4 in a (GGGGS)ₙ linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)ₙ sequence also comprises an N-terminal threonine. In some embodiments, the linker is any one of the following:

(SEQ ID NO: 21)
GGGGSGGGGS
(SEQ ID NO: 4)
TGGGGSGGGGS
(SEQ ID NO: 5)
TGGGGSGGGGSGGGGS
(SEQ ID NO: 6)
TGGGGSGGGGSGGGGSGGGGS
(SEQ ID NO: 25)
TGGGGSGGGGSGGGGSGGGGSGGGGS
(SEQ ID NO: 26)
TGGGGSGGGGSGGGGSGGGGSGGGGSGGGGS
or
(SEQ ID NO: 40)
TGGGGSGGGGSGGGGSGGGGSGGGGSGGGGSGGGGS.

In some embodiments, the linker comprises the amino acid sequence of TGGGPKSCDK (SEQ ID NO: 7). In some embodiments, the linker is any one of SEQ ID NOs: 21, 4-7, 25-26 or 40 lacking the N-terminal threonine. In some embodiments, a linker may be rich in glycine (e.g., 2-10, 2-5, 2-4, 2-3 glycine residues) and may, for example, contain a single sequence of threonine/serine and glycines or repeating sequences of threonine/serine and/or glycines, e.g., GGG (SEQ ID NO: 63), GGGG (SEQ ID NO: 64), TGGGG (SEQ ID NO: 65), SGGGG (SEQ ID NO: 66), or SGGG (SEQ ID NO: 67) singlets, or repeats. In some embodiments, the linker does not comprise the amino acid sequence of SEQ ID NO: 26 or 40.

In certain aspects, functional variants or modified forms of the polypeptides disclosed herein include fusion proteins having at least a portion of the polypeptide (e.g., an activin antagonist polypeptide, a TGFβ antagonist polypeptide, or immune checkpoint antagonist polypeptide) and one or more heterologous portions. Well-known examples of such heterologous portions include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A heterologous portion may be selected so as to confer a desired property. For example, some heterologous portions are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a heterologous portion may be selected so as to facilitate detection of the polypeptide (e.g., an activin antagonist polypeptide, a TGFβ antagonist polypeptide, or immune checkpoint antagonist polypeptide). Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the heterologous portions have a protease cleavage site, such as for Factor Xa or Thrombin, which allows the relevant protease to partially digest the fusion proteins and thereby liberate the recombinant proteins therefrom. The liberated proteins can then be isolated from the heterologous portion by subsequent chromatographic separation. In certain preferred embodiments, a polypeptide of the disclosure (e.g., an activin antagonist polypeptide, a TGFβ antagonist polypeptide, or immune checkpoint antagonist polypeptide) is fused with a domain that stabilizes the polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of heterologous portions that may be selected include multimerizing (e.g., dimerizing, tetramerizing) domains and functional domains.

It is understood that different elements of the fusion proteins may be arranged in any manner that is consistent with the desired functionality. For example, an activin antagonist polypeptide, a TGFβ antagonist polypeptide, or immune checkpoint antagonist polypeptide may be placed C-terminal to a heterologous domain, or, alternatively, a heterologous domain may be placed C-terminal to an activin antagonist polypeptide, a TGFβ antagonist polypeptide, or immune checkpoint antagonist polypeptide. The activin antagonist polypeptide, TGFβ antagonist polypeptide, or immune checkpoint antagonist polypeptide domain and the heterologous domain need not be adjacent in a fusion protein, and additional domains or amino acid sequences may be included C- or N-terminal to either domain or between the domains.

As used herein, the term “immunoglobulin Fc domain” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain. In some embodiments, the immunoglobulin Fc region is a human immunoglobulin Fc region.

In one embodiment, the class of immunoglobulin from which the heavy chain constant region is derived is IgG (Igγ) (γ subclasses 1, 2, 3, or 4).

An example of a native amino acid sequence that may be used for the Fc portion of human IgG1 (G1Fc) is shown below (SEQ ID NO: 58). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 58. Naturally occurring variants in G1Fc would include E134D and M136L according to the numbering system used in SEQ ID NO: 58 (see Uniprot P01857).

(SEQ ID NO: 58)

| | |
|---|---|
| 1 | 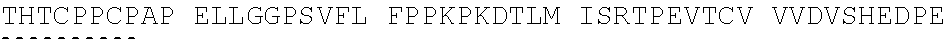 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 59). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 59.

(SEQ ID NO: 59)

| | |
|---|---|
| 1 | 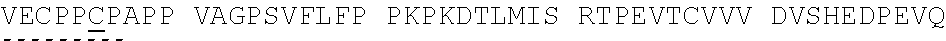 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 60) contains a short hinge region consisting of a single 15-residue segment, whereas the second G3Fc sequence (SEQ ID NO: 61) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 60 or 61.

(SEQ ID NO: 60)

| | |
|---|---|
| 1 | 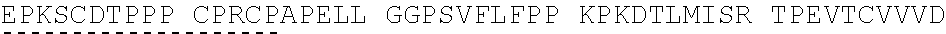 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |

(SEQ ID NO: 61)

| | |
|---|---|
| 1 | 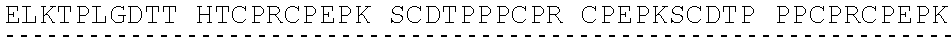 |
| 51 | 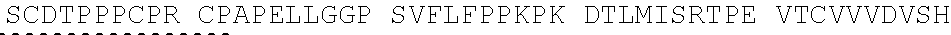 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 60, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 62). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 62.

(SEQ ID NO: 62)

| | |
|---|---|
| 1 | 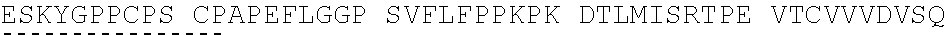 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

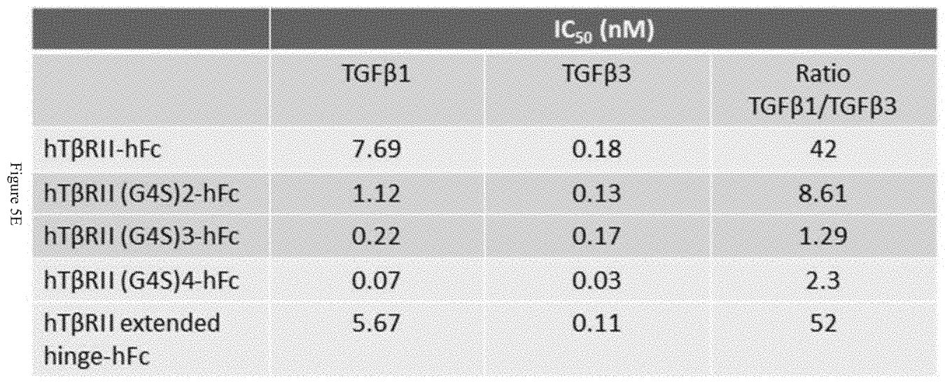

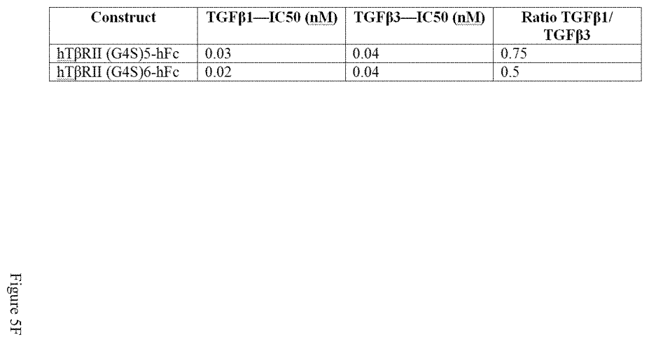

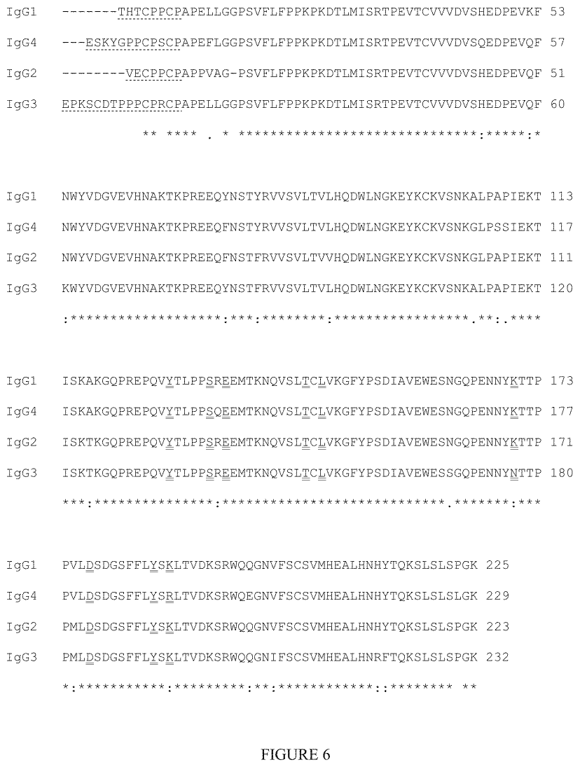

A variety of engineered mutations in the Fc domain are presented herein with respect to the G1Fc sequence (SEQ ID NO: 58), and analogous mutations in G2Fc, G3Fc, and G4Fc can be derived from their alignment with G1Fc in FIG. 6. Due to unequal hinge lengths, analogous Fc positions based on isotype alignment (FIG. 6) possess different amino acid numbers in SEQ ID NOs: 58, 59, 60, 61, and 62. It can also be appreciated that a given amino acid position in an immunoglobulin sequence consisting of hinge, C_{H}2, and C_{H}3 regions (e.g., SEQ ID NOs: 58, 59, 60, 61, and 62) will be identified by a different number than the same position when numbering encompasses the entire IgG1 heavy-chain constant domain (consisting of the C_{H}1, hinge, C_{H}2, and C_{H}3 regions) as in the Uniprot database.

Other classes of immunoglobulin, IgA (Igα), IgD (Igδ), IgE (Igε) and IgM (Igμ), may be used. The choice of appropriate immunoglobulin heavy chain constant region is discussed in detail in U.S. Pat. Nos. 5,541,087 and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc gamma or the homologous domains in any of IgA, IgD, IgE, or IgM.

Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al. (1997) J. Immunol. 159:3613).

For example, the application further provides Fc fusion proteins with engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus, in certain embodiments, Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

In certain preferred embodiments, bi- or tri-functional fusion proteins of the disclosure are heteromultimers comprising at least two or more polypeptide domains selected from an activin antagonist polypeptide, a TGFβ antagonist polypeptide, or an immune checkpoint antagonist polypeptide, wherein the two or more polypeptide domains are associated, covalently or non-covalently. In some embodiments, such bi- or tri-functional fusion protein heteromultimers are heterodimeric complexes, although higher order heteromultimeric complexes are also included such as, but not limited to, heterotrimers, heterotetramers, and further oligomeric structures. In some embodiments, bi- or tri-functional fusion proteins of the disclosure comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a first polypeptide and a second polypeptide to promote heteromultimer formation (e.g., heterodimer formation), and optionally hinders or otherwise disfavors homomultimer formation (e.g., homodimer formation), thereby increasing the yield of desired heteromultimer.

Many methods known in the art can be used to generate protein heteromultimers. For example, non-naturally occurring disulfide bonds may be constructed by replacing on a first polypeptide (a naturally occurring amino acid with a free thiol-containing residue, such as cysteine, such that the free thiol interacts with another free thiol-containing residue on a second polypeptide (such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kalman et al., U.S. Pat. No. 8,592,562; coiled-coil interactions such as described in Christensen et al., U.S.20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

The first and second members of the interaction pair may be an asymmetric pair, meaning that the members of the pair preferentially associate with each other rather than self-associate. Accordingly, first and second members of an asymmetric interaction pair may associate to form a heterodimeric complex. Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

A problem that arises in large-scale production of asymmetric immunoglobulin-based proteins from a single cell line is known as the “chain association issue”. As confronted prominently in the production of bispecific antibodies, the chain association issue concerns the challenge of efficiently producing a desired multichain protein from among the multiple combinations that inherently result when different heavy chains and/or light chains are produced in a single cell line [Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605]. As described herein, these methods may be used to generate ActRIIB-Fc:TβRII-Fc heteromultimer.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation.

Examples of Pair-Wise Charged Residue Mutations
to Enhance Heterodimer Formation

| | | Interacting | Corresponding |
|---|---|---|---|
| Position in | Mutation in | position in | mutation in |
| first chain | first chain | second chain | second chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments, one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to a first polypeptide or second polypeptide of the construct, with or without an optional linker, to generate a heteromultimer. This single chain can be coexpressed in a cell of choice along with the Fc sequence complementary to the first Fc to favor generation of the desired multichain construct. In this example based on electrostatic steering, SEQ ID NO: 68 [human G1Fc(E134K/D177K)] and SEQ ID NO: 69 [human G1Fc(K170D/K187D)] are examples of complementary Fc sequences in which the engineered amino acid substitutions are double underlined, and the first polypeptide or the second polypeptide of the construct can be fused to either SEQ ID NO: 68 or SEQ ID NO: 69, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 68 and 69).

| (SEQ ID NO: 68) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSRKEMTKNQ VSLTCLVKGF |
| 151 | YPSDIAVEWE SNGQPENNYK TTPPVLKSDG SFFLYSKLTV DKSRWQQGNV |
| 201 | FSCSVMHEAL HNHYTQKSLS LSPGK |
| (SEQ ID NO: 69) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYD TTPPVLDSDG SFFLYSDLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to a first polypeptide or a second polypeptide of the construct, with or without an optional linker, to generate a heteromultimer. This single chain can be co-expressed in a cell of choice along with the Fc sequence complementary to the first Fc to favor generation of the desired multi-chain construct. In this example based on knobs-into-holes pairing, SEQ ID NO: 70 [human G1Fc(T144Y)] and SEQ ID NO: 71 [human G1Fc(Y185T)] are examples of complementary Fc sequences in which the engineered amino acid substitutions are double underlined, and the TβRII or ActRIIB polypeptide of the construct can be fused to either SEQ ID NO: 70 or SEQ ID NO: 71, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 70 and 71).

| (SEQ ID NO: 70) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLYCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 71) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLTSKLTV DKSRWQQGNV |
| 201 | FSCSVMHEAL HNHYTQKSLS LSPGK |

An example of Fc complementarity based on knobs-into-holes pairing combined with an engineered disulfide bond is disclosed in SEQ ID NO: 72 [hG1Fc(S132C/T144W)] and SEQ ID NO: 73 [hG1Fc(Y127C/T144S/L146A/Y185V)]. The engineered amino acid substitutions in these sequences are double underlined, and the TGFβ superfamily type I or type II polypeptide of the construct can be fused to either SEQ ID NO: 72 or SEQ ID NO: 73, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 72 and 73).

| (SEQ ID NO: 72) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 73) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SNGQPENNYK TTPPVLDSDG SFFLVSKLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [Davis et al. (2010) Protein Eng Design Sel 23:195-202]. One of a pair of Fc sequences with SEEDbody complementarity can be arbitrarily fused to a first polypeptide or a second polypeptide of the construct, with or without an optional linker, to generate a first polypeptide or second polypeptide fusion protein. This single chain can be co-expressed in a cell of choice along with the Fc sequence complementary to the first Fc to favor generation of the desired multi-chain construct. In this example based on SEEDbody (Sb) pairing, SEQ ID NO: 74 [hG1Fc(Sb_{AG})] and SEQ ID NO: 75 [hG1Fc(Sb_{GA})] are examples of complementary IgG Fc sequences in which the engineered amino acid substitutions from IgA Fc are double underlined, and the first polypeptide or the second polypeptide of the construct can be fused to either SEQ ID NO: 74 or SEQ ID NO: 75, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 6) will generate an Fc monomer which may be used in the complementary IgG-IgA pair below (SEQ ID NOs: 74 and 75).

| (SEQ ID NO: 74) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PFRPEVHLLP PSREEMTKNQ VSLTCLARGF |
| 151 | YPKDIAVEWE SNGQPENNYK TTPSRQEPSQ GTTTFAVTSK LTVDKSRWQQ |
| 201 | GNVFSCSVMH EALHNHYTQK TISLSPGK |
| (SEQ ID NO: 75) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PPSEELALNE LVTLTCLVKG |
| 151 | FYPSDIAVEW ESNGQELPRE KYLTWAPVLD SDGSFFLYSI LRVAAEDWKK |
| 201 | GDTFSCSVMH EALHNHYTQK SLDRSPGK |

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains [Wranik et al (2012) J Biol Chem 287:43331-43339]. As disclosed herein, one of a pair of Fc sequences attached to a leucine zipper-forming strand can be arbitrarily fused to a first polypeptide or a second polypeptide of the construct, with or without an optional linker, to generate a first polypeptide or second polypeptide fusion protein. This single chain can be co-expressed in a cell of choice along with the Fc sequence attached to a complementary leucine zipper-forming strand to favor generation of the desired multi-chain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 76 [hG1Fc-Ap1 (acidic)] and SEQ ID NO: 77 [hG1Fc-Bp1 (basic)] are examples of complementary IgG Fc sequences in which the engineered complimentary leucine zipper sequences are underlined, and the first polypeptide or the second polypeptide of the construct can be fused to either SEQ ID NO: 76 or SEQ ID NO: 77, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc (see FIG. 6) will generate an Fc monomer which may be used in the complementary leucine zipper-forming pair below (SEQ ID NOs: 76 and 77).

| (SEQ ID NO: 76) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LEKELQALEK ENAQLEWELQ |
| 251 | ALEKELAQGA T |
| (SEQ ID NO: 77) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LKKKLQALKK KNAQLKWKLQ |
| 251 | ALKKKLAQGA T |

In certain aspects, the disclosure relates to a first polypeptide comprising one or more amino acid modifications that alter the isoelectric point (pI) of the first polypeptide and/or a second polypeptide comprising one or more amino acid modifications that alter the isoelectric point of the second polypeptide. In some embodiments, one or more candidate domains that have a pI value higher than about 5.0, 5.5, 6.0, 6.5, 7.0, 7.5, 8.0, 8.5, or 9.0 are selected for construction of the full multidomain protein. In other embodiments, one or more candidate domains that have a pI value less than about 9.0, 8.5, 8.0, 7.5, 7.0, 6.5, 6.0, 5.5, or 5.0 are selected for construction of the full multidomain protein. It will be understood by one skilled in the art that a single protein will have multiple charge forms. Without wishing to be bound by any particular theory, the charge of a protein can be modified by a number of different mechanisms including but not limited to, amino acid substitution, cationization, deamination, carboxyl-terminal amino acid heterogeneity, phosphorylation and glycosylation.

The pI of a protein may be determined by a variety of methods including but not limited to, isoelectric focusing and various computer algorithms (see for example Bjellqvist et al., 1993, Electrophoresis 14:1023). In one embodiment, pI is determined using a Pharmacia Biotech Multiphor 2 electrophoresis system with a multi temp refrigerated bath recirculation unit and an EPS 3501 XL power supply. Pre-cast ampholine gels (e.g., Amersham Biosciences, pI range 2.5-10) are loaded with protein samples. Broad range pI marker standards (e.g., Amersham, pI range 3-10, 8 .mu.L) are used to determine relative pI for the proteins. Electrophoresis may be performed, for example, at 1500 V, 50 mA for 105 minutes. The gel is fixed using, for example, a Sigma fixing solution (5×) diluted with purified water to 1× Staining is performed, for example, overnight at room temperature using Simply Blue stain (Invitrogen). Destaining is carried out, for example, with a solution that consisted of 25% ethanol, 8% acetic acid and 67% purified water. Isoelectric points are determined using, for example, a Bio-Rad Densitometer relative to calibration curves of the standards. The one or more metrics may further include metrics characterizing stability of the domain under one or more different conditions selected from the group consisting of different pH values, different temperatures, different shear stresses, and different freeze/thaw cycles.

In part, the disclosure provides desired pairing of asymmetric Fc-containing polypeptide chains by methods described above in combination with additional mutations in the Fc domain which facilitate purification of the desired heteromeric species. An example is complementarity of Fc domains based on knobs-into-holes pairing combined with an engineered disulfide bond, as disclosed in SEQ ID NOs: 72-73, plus additional substitution of two negatively charged amino acids (aspartic acid or glutamic acid) in one Fc-containing polypeptide chain and two positively charged amino acids (e.g., arginine) in the complementary Fc-containing polypeptide chain (SEQ ID NOs: 78-79). These four amino acid substitutions facilitate selective purification of the desired heteromeric fusion protein from a heterogeneous polypeptide mixture based on differences in isoelectric point or net molecular charge. The engineered amino acid substitutions in these sequences are double underlined below, and a first polypeptide or a second polypeptide of the construct can be fused to either SEQ ID NO: 78 or SEQ ID NO: 79, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 78-79).

| (SEQ ID NO: 78) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTENQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQDSLS LSPGK |
| (SEQ ID NO: 79) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SRGQPENNYK TTPPVLDSRG SFFLVSKLTV DKSRWQQGNV |
| 201 | FSCSVMHEAL HNHYTQKSLS LSPGK |

Another example involves complementarity of Fc domains based on knobs-into-holes pairing combined with an engineered disulfide bond, as disclosed in SEQ ID NOs: 72-73, plus a histidine-to-arginine substitution at position 213 in one Fc-containing polypeptide chain (SEQ ID NO: 80). This substitution (denoted H435R in the numbering system of Kabat et al.) facilitates separation of desired heteromer from undesirable homodimer based on differences in affinity for protein A. The engineered amino acid substitution is indicated by double underline, and the first polypeptide or the second polypeptide of the construct can be fused to either SEQ ID NO: 80 or SEQ ID NO: 73, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see Figure: 6) will generate complementary Fc pairs which may be used instead of the complementary hG1Fc pair of SEQ ID NO: 80 (below) and SEQ ID NO: 73.

| (SEQ ID NO: 80) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNRYTQKSLS LSPGK |

### 3. Nucleic Acids and Methods of Manufacture

In certain embodiments, the present disclosure makes available isolated and/or purified forms of polypeptides, as well as bi- or tri-functional fusion proteins, homodimer, and heterodimers comprising the same, which are isolated from, or otherwise substantially free of (e.g., at least 80%, 85%, 90%, 91%, 92%, 93%, 94%, 95%, 96%, 97%, 98%, or 99% free of), other proteins and/or other polypeptide species. Polypeptides will generally be produced by expression from recombinant nucleic acids. Thus, polypeptides of the disclosure, as well as bi- or tri-functional fusion proteins, homodimer, and heterodimers comprising the same, will generally be recombinant.

In certain embodiments, the disclosure includes nucleic acids encoding soluble polypeptides, as well as bi- or tri-functional fusion proteins, homodimer, and heterodimers comprising the same, comprising the coding sequence for a portion of a protein (e.g., an extracellular, ligand-binding domain of a receptor or a ligand-binding domain of an antibody). In further embodiments, this disclosure also pertains to a host cell comprising such nucleic acids. The host cell may be any prokaryotic or eukaryotic cell. For example, a polypeptide of the present disclosure may be expressed in bacterial cells such as E. coli, insect cells (e.g., using a baculovirus expression system), yeast, or mammalian cells. Other suitable host cells are known to those skilled in the art. Accordingly, some embodiments of the present disclosure further pertain to methods of producing the polypeptides.

In certain aspects, the disclosure provides isolated and/or recombinant nucleic acids encoding any of the polypeptides, as well as bi- or tri-functional fusion proteins, homodimer, and heterodimers comprising the same, including fragments, functional variants and fusion proteins disclosed herein. SEQ ID NOs: 8, 10, 12, 14, 16, 46, 47, 56, 57, 83, 86, 89, 92, 113, 114, 122, 123, 126, 127, 129, 132, 135, 138, and 142 encode TβRII, ActRIIA, ActRIIB or betaglycan polypeptides as well as variants thereof comprising an extracellular domain fused to an IgG Fc domain. The subject nucleic acids may be single-stranded or double stranded. Such nucleic acids may be DNA or RNA molecules. These nucleic acids may be used, for example, in methods for making polypeptides or as direct therapeutic agents (e.g., in an antisense, RNAi or gene therapy approach).

In certain aspects, the subject nucleic acids encoding polypeptides are further understood to include nucleic acids that are variants of SEQ ID NOs: 8, 10, 12, 14, 16, 46, 47, 56, 57, 83, 86, 89, 92, 113, 114, 122, 123, 126, 127, 129, 132, 135, 138, and 142. Variant nucleotide sequences include sequences that differ by one or more nucleotide substitutions, additions or deletions, such as allelic variants.

In certain embodiments, the disclosure provides isolated or recombinant nucleic acid sequences that are at least 70%, 75%, 80%, 85%, 90%, 91%, 92%, 93%, 94%, 95%, 96%, 97%, 98%, 99% or 100% identical to SEQ ID NOs: 8, 10, 12, 14, 16, 46, 47, 56, 57, 83, 86, 89, 92, 113, 114, 122, 123, 126, 127, 129, 132, 135, 138, and 142. One of ordinary skill in the art will appreciate that nucleic acid sequences complementary to SEQ ID NOs: 8, 10, 12, 14, 16, 46, 47, 56, 57, 83, 86, 89, 92, 113, 114, 122, 123, 126, 127, 129, 132, 135, 138, and 142, and variants of SEQ ID NOs: 8, 10, 12, 14, 16, 46, 47, 56, 57, 83, 86, 89, 92, 113, 114, 122, 123, 126, 127, 129, 132, 135, 138, and 142 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to the nucleotide sequences designated in SEQ ID NOs: 8, 10, 12, 14, 16, 46, 47, 56, 57, 83, 86, 89, 92, 113, 114, 122, 123, 126, 127, 129, 132, 135, 138, and 142 complement sequences of SEQ ID NOs: 8, 10, 12, 14, 16, 46, 47, 56, 57, 83, 86, 89, 92, 113, 114, 122, 123, 126, 127, 129, 132, 135, 138, and 142, or fragments thereof. As discussed above, one of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In some embodiments, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 8, 10, 12, 14, 16, 46, 47, 56, 57, 83, 86, 89, 92, 113, 114, 122, 123, 126, 127, 129, 132, 135, 138, and 142 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

It will be appreciated by one of skill in the art that corresponding variants based on the long isoform of TβRII will include nucleotide sequences encoding the 25-amino acid insertion along with a conservative Val-Ile substitution at the flanking position C-terminal to the insertion. It will also be appreciated that corresponding variants based on either the long (A) or short (B) isoforms of TβRII will include variant nucleotide sequences comprising an insertion of 108 nucleotides, encoding a 36-amino-acid insertion (SEQ ID NO: 41), at the same location described for naturally occurring TβRII isoform C.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects disclosed herein, the subject nucleic acid is provided in an expression vector comprising a nucleotide sequence encoding a polypeptide (e.g., bi- or tri-functional fusion proteins) and operably linked to at least one regulatory sequence. Regulatory sequences are art-recognized and are selected to direct expression of the T polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a polypeptide (e.g., bi- or tri-functional fusion proteins). Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid included in the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide (e.g., bi- or tri-functional fusion proteins) include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

In certain embodiments, a vector will be designed for production of the subject polypeptides (e.g., bi- or tri-functional fusion proteins) in CHO cells, such as a Pcmv-Script vector (Stratagene, La Jolla, Calif.), pcDN4 vectors (Invitrogen, Carlsbad, Calif.) and pCI-neo vectors (Promega, Madison, Wis.). In a preferred embodiment, a vector will be designed for production of the subject polypeptides in HEK-293 cells. As will be apparent, the subject gene constructs can be used to cause expression of the subject polypeptides in cells propagated in culture, e.g., to produce proteins, including fusion proteins or variant proteins, for purification.

This disclosure also pertains to a host cell transfected with a recombinant gene including a coding sequence (e.g., SEQ ID NOs: 8, 10, 12, 14, 16, 46, 47, 56, 57, 83, 86, 89, 92, 113, 114, 122, 123, 126, 127, 129, 132, 135, 138, and 142) for one or more of the subject polypeptides (e.g., bi- or tri-functional fusion proteins). The host cell may be any prokaryotic or eukaryotic cell. For example, a bi- or tri-functional fusion protein disclosed herein may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject polypeptides (e.g., bi- or tri-functional fusion proteins). For example, a host cell transfected with an expression vector encoding a polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically or in a membrane fraction and the cells harvested, lysed and the protein isolated. A cell culture includes host cells, and media. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the polypeptides and affinity purification with an agent that binds to a domain fused to the polypeptide (e.g., a protein A column may be used to purify an Fc fusion). In a preferred embodiment, the polypeptide is a fusion protein containing a domain which facilitates its purification. As an example, purification may be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

In another embodiment, a fusion gene coding for a purification leader sequence, such as a poly-(His)/enterokinase cleavage site sequence at the N-terminus of the desired portion of the recombinant polypeptide (e.g., bi- or tri-functional fusion proteins), can allow purification of the expressed fusion protein by affinity chromatography using a Ni²⁺ metal resin. The purification leader sequence can then be subsequently removed by treatment with enterokinase to provide the purified polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 4. Antibody Antagonists

In certain aspects, the present disclosure relates to bi- or tri-functional fusion proteins comprising two or more of an activin antagonist domain, a TGFβ antagonist domain, and an immune checkpoint antagonist domain, wherein one or more of the activin antagonist domain, the TGFβ antagonist domain, and the immune checkpoint antagonist domain is an antibody, or antigen-binding domain thereof, that binds to one or more of activin (e.g., activin A, activin B, activin C, activin E, activin AB, activin AC), TGFβ (e.g., TGFβ L TGFβ2, and/or TGFβ3), ActRII (e.g., ActRIIA and/or ActRIIB), TβRII, betaglycan, and an immune checkpoint inhibitor (e.g., PD-1, PD-L1, CTLA4, BTLA, LAG3, TIM3, LAIR1, B7-DC, HVEM, TIM4, B7-H3, and/or B7-H4). In particular, such bi- or tri-functional fusion proteins comprising an antibody, or antigen-binding domain thereof, that binds to one or more of activin, TGFβ, TβRII, betaglycan, and an immune checkpoint inhibitor, may be used to treat or prevent one or more diseases or conditions described herein (e.g., cancer, tumors, pre-neoplastic disorders, hyperproliferative disorders, and dysplastic disorders).

In some embodiments, an activin antagonist domain is an antibody, or antigen-binding domain thereof, that binds to activin (e.g., activin A, activin B, activin C, activin E, activin AB, and/or activin AC). As used herein, an activin antibody (anti-activin antibody) generally refers to an antibody that binds to activin with sufficient affinity such that the antibody is useful as a diagnostic and/or therapeutic agent in targeting activin. In certain embodiments, the extent of binding of an anti-activin antibody to an unrelated, non-activin protein is less than about 10%, 9%, 8%, 7%, 6%, 5%, 4%, 3%, 2%, or less than about 1% of the binding of the antibody to activin as measured, for example, by a radioimmunoassay (RIA), Biacore, or other protein-protein interaction or binding affinity assay. In certain embodiments, an anti-activin antibody binds to an epitope of activin that is conserved among activin from different species. In certain preferred embodiments, an anti-activin antibody binds to human activin. In other preferred embodiments, an anti-activin antibody may inhibit activin from binding to a cognate type I and/or type II receptor (e.g., ActRIIA, ActRIIB, and ALK4) and thus inhibit activin-mediated signaling (e.g., Smad signaling) via these receptors. It should be noted that activins share sequence homology and therefore antibodies that bind to one activin (e.g., activin A) may bind to one or more additional activins (e.g., activin B, activin AB, activin C, activin E, activin AC). In some embodiments, an anti-activin antibody binds to at least activin A and/or activin B. Examples of activin antibodies are disclosed in International Patent Application Publication No. WO 2015/017576, which is incorporated herein by reference in their entirety. Any of these antibodies, or antigen-binding fragments thereof, may be incorporated into the bi- or tri-functional fusion proteins as well as methods of the instant disclosure. In some embodiments, the activin antibody is REGN2477, or an antigen-binding fragment thereof.

In some embodiments, an activin antagonist domain is an antibody, or antigen-binding domain thereof, that binds to an ActRII receptor (e.g., ActRIIA and/or ActRIIB). As used herein, an ActRII antibody (anti-ActRII antibody) generally refers to an antibody that binds to ActRII (e.g., ActRIIA and/or ActRIIB) with sufficient affinity such that the antibody is useful as a diagnostic and/or therapeutic agent in targeting ActRII. In some embodiments, an ActRII antibody binds to ActRIIA, but does not bind or does not substantially bind to ActRIIB (e.g., binds to ActRIIB with a K_{D} of greater than 1×10⁻⁷ M or has relatively modest binding, for example, about 1×10⁻⁸ M or about 1×10⁻⁹ M). In other embodiments, an ActRII antibody binds to ActRIIB, but does not bind or does not substantially bind to ActRIIA (e.g., binds to ActRIIA with a K_{D} of greater than 1×10⁻⁷ M or has relatively modest binding, for example, about 1×10⁻⁸ M or about 1×10⁻⁹ M). In still other embodiments, an ActRII antibody binds to ActRIIA and ActRIIB. In certain embodiments, the extent of binding of an anti-ActRII antibody to an unrelated, non-ActRII protein is less than about 10%, 9%, 8%, 7%, 6%, 5%, 4%, 3%, 2%, or less than about 1% of the binding of the antibody to ActRII as measured, for example, by a radioimmunoassay (RIA), Biacore, or other protein-protein interaction or binding affinity assay. In certain embodiments, an anti-ActRII antibody binds to an epitope of ActRII (e.g., ActRIIA and/or ActRIIB) that is conserved among ActRII from different species. In certain preferred embodiments, an anti-ActRII antibody binds to human ActRII (e.g., ActRIIA and/or ActRIIB). It should be noted that ActRIIA has sequence homology to ActRIIB and therefore antibodies that bind to ActRIIA, in some cases, may also bind to and/or inhibit ActRIIB, the reverse is also true. Examples of such ActRIIB and/or ActRIIA antibodies are disclosed in International Patent Application Publication Nos. WO 2012/064771, WO 2013/063536, WO 2017/156488, WO 2010/125003, and WO 2013/188448, which are incorporated herein by reference in their entirety. Any of these antibodies, or antigen-binding fragments thereof, may be incorporated into the bi- or tri-functional fusion proteins as well as methods of the instant disclosure. In some embodiments, the ActRIIA/B antibody is bimagrumab, or an antigen-binding fragment thereof.

In some embodiments, a TGFβ antagonist domain is an antibody, or antigen-binding domain thereof, that binds to TGFβ (e.g., TGFβ L TGFβ 2, and/or TGFβ3). As used herein, a TGFβ antibody (anti-TGFβ antibody) generally refers to an antibody that binds to TGFβ with sufficient affinity such that the antibody is useful as a diagnostic and/or therapeutic agent in targeting TGFβ. In certain embodiments, the extent of binding of an anti-TGFβ antibody to an unrelated, non-activin protein is less than about 10%, 9%, 8%, 7%, 6%, 5%, 4%, 3%, 2%, or less than about 1% of the binding of the antibody to TGFβ as measured, for example, by a radioimmunoassay (RIA), Biacore, or other protein-protein interaction or binding affinity assay. In certain embodiments, an anti-TGFβ antibody binds to an epitope of TGFβ that is conserved among TGFβ from different species. In certain preferred embodiments, an anti-TGFβ antibody binds to human TGFβ. In other preferred embodiments, an anti-TGFβ antibody may inhibit TGFβ from binding to a cognate type I, type II, or co-receptor (e.g., TβRII, ALK5, and betaglycan) and thus inhibit TGFβ-mediated signaling (e.g., Smad signaling) via these receptors. It should be noted that TGFβ share sequence homology and therefore antibodies that bind to one TGFβ (e.g., TGFβ1) may bind to one or more additional TGFβ s (e.g., TGFβ 2 and/or TGFβ3). In some embodiments, an anti-TGFβ antibody binds to TGFβ L TGFβ2, and TGFβ3. In alternative embodiments, an anti-TGFβ antibody binds to TGFβ1 and TGFβ3, but does not bind or does not substantially bind to TGFβ2 (e.g., binds to TGFβ2 with a K_{D} of greater than 1×10⁻⁷ M or has relatively modest binding, for example, about 1×10⁻⁸ M or about 1×10⁻⁹ M). In some embodiments, the TGFβ antibody is fresolimumab, or an antigen-binding fragment thereof. In some embodiments, the TGFβ antibody is metelimumab, or an antigen-binding fragment thereof.

In some embodiments, a TGFβ antagonist domain is an antibody, or antigen-binding domain thereof, that binds to TβRII. As used herein, a TβRII antibody (anti-TβRII antibody) generally refers to an antibody that binds to TβRII with sufficient affinity such that the antibody is useful as a diagnostic and/or therapeutic agent in targeting TβRII. In certain embodiments, the extent of binding of an anti-TβRII antibody to an unrelated, non-TβRII protein is less than about 10%, 9%, 8%, 7%, 6%, 5%, 4%, 3%, 2%, or less than about 1% of the binding of the antibody to TβRII as measured, for example, by a radioimmunoassay (RIA), Biacore, or other protein-protein interaction or binding affinity assay. In certain embodiments, an anti-TβRII antibody binds to an epitope of TβRII that is conserved among TβRII from different species. In certain preferred embodiments, an anti-TβRII antibody binds to human TβRII.

In some embodiments, a TGFβ antagonist domain is an antibody, or antigen-binding domain thereof, that binds to betaglycan. As used herein, a betaglycan antibody (anti-betaglycan antibody) generally refers to an antibody that binds to betaglycan with sufficient affinity such that the antibody is useful as a diagnostic and/or therapeutic agent in targeting betaglycan. In certain embodiments, the extent of binding of an anti-betaglycan antibody to an unrelated, non-betaglycan protein is less than about 10%, 9%, 8%, 7%, 6%, 5%, 4%, 3%, 2%, or less than about 1% of the binding of the antibody to betaglycan as measured, for example, by a radioimmunoassay (RIA), Biacore, or other protein-protein interaction or binding affinity assay. In certain embodiments, an anti-betaglycan antibody binds to an epitope of betaglycan that is conserved among betaglycan from different species. In certain preferred embodiments, an anti betaglycan antibody binds to human betaglycan.

In some embodiments, an immune checkpoint antagonist domain is an antibody, or antigen-binding domain thereof, that binds to PD-1. As used herein, a PD-1 antibody (anti-PD-1 antibody) generally refers to an antibody that binds to PD-1 with sufficient affinity such that the antibody is useful as a diagnostic and/or therapeutic agent in targeting PD-1 (e.g., inhibit PD-1-mediated immune suppression activities). In certain embodiments, the extent of binding of an anti-PD-1 antibody to an unrelated, non-betaglycan protein is less than about 10%, 9%, 8%, 7%, 6%, 5%, 4%, 3%, 2%, or less than about 1% of the binding of the antibody to PD-1 as measured, for example, by a radioimmunoassay (RIA), Biacore, or other protein-protein interaction or binding affinity assay. In certain embodiments, an anti-PD-1 antibody binds to an epitope of PD-1 that is conserved among PD-1 from different species. In certain preferred embodiments, an anti-PD-1 antibody binds to human PD-1. In other preferred embodiments, an anti-PD-1 antibody may inhibit PD-1 from binding to PD-L1. In some embodiments, the anti-PD-1 antibody is nivolumab, or an antigen-binding fragment thereof. In some embodiments, the anti-PD-1 antibody is pembrolizumab, or an antigen-binding fragment thereof.

In some embodiments, an immune checkpoint antagonist domain is an antibody, or antigen-binding domain thereof, that binds to PD-L1. As used herein, a PD-L1 antibody (anti-PD-L1 antibody) generally refers to an antibody that binds to PD-L1 with sufficient affinity such that the antibody is useful as a diagnostic and/or therapeutic agent in targeting PD-L1 (e.g., inhibit PD-L1-mediated immune suppression activities). In certain embodiments, the extent of binding of an anti-PD-L1 antibody to an unrelated, non-betaglycan protein is less than about 10%, 9%, 8%, 7%, 6%, 5%, 4%, 3%, 2%, or less than about 1% of the binding of the antibody to PD-L1 as measured, for example, by a radioimmunoassay (RIA), Biacore, or other protein-protein interaction or binding affinity assay. In certain embodiments, an anti-PD-L1 antibody binds to an epitope of PD-L1 that is conserved among PD-L1 from different species. In certain preferred embodiments, an anti-PD-L 1 antibody binds to human PD-L1. In other preferred embodiments, an anti-PD-L1 antibody may inhibit PD-L1 from binding to PD-1. In some embodiments, the anti-PD-L1 antibody is atezolizumab, or an antigen-binding fragment thereof. In some embodiments, the anti-PD-L1 antibody is avelumab, or an antigen-binding fragment thereof. In some embodiments, the anti-PD-L1 antibody is durvalumab, or an antigen-binding fragment thereof.

In some embodiments, an immune checkpoint antagonist domain is an antibody, or antigen-binding domain thereof, that binds to CTLA4. As used herein, a CTLA4 antibody (anti-CTLA4 antibody) generally refers to an antibody that binds to CTLA4 with sufficient affinity such that the antibody is useful as a diagnostic and/or therapeutic agent in targeting CTLA4 (e.g., inhibit CTLA4-mediated immune suppression activities). In certain embodiments, the extent of binding of an anti-CTLA4 antibody to an unrelated, non-betaglycan protein is less than about 10%, 9%, 8%, 7%, 6%, 5%, 4%, 3%, 2%, or less than about 1% of the binding of the antibody to CTLA4 as measured, for example, by a radioimmunoassay (RIA), Biacore, or other protein-protein interaction or binding affinity assay. In certain embodiments, an anti-CTLA4 antibody binds to an epitope of CTLA4 that is conserved among CTLA4 from different species. In certain preferred embodiments, an anti-CTLA4antibody binds to human CTLA4. In other preferred embodiments, an anti-CTLA4antibody may inhibit CTLA4 from binding to MHC class II molecules. In some embodiments, the anti-PD-L1 antibody is ipilimumab, or an antigen-binding fragment thereof.

In some embodiments, an immune checkpoint antagonist domain is an antibody, or antigen-binding domain thereof, that binds to BTLA. As used herein, a BTLA antibody (anti-BTLA antibody) generally refers to an antibody that binds to BTLA with sufficient affinity such that the antibody is useful as a diagnostic and/or therapeutic agent in targeting BTLA (e.g., inhibit BTLA-mediated immune suppression activities). In certain embodiments, the extent of binding of an anti-BTLA antibody to an unrelated, non-betaglycan protein is less than about 10%, 9%, 8%, 7%, 6%, 5%, 4%, 3%, 2%, or less than about 1% of the binding of the antibody to BTLA as measured, for example, by a radioimmunoassay (RIA), Biacore, or other protein-protein interaction or binding affinity assay. In certain embodiments, an anti-BTLA antibody binds to an epitope of BTLA that is conserved among BTLA from different species. In certain preferred embodiments, an anti-BTLA antibody binds to human BTLA.

In some embodiments, an immune checkpoint antagonist domain is an antibody, or antigen-binding domain thereof, that binds to LAG3. As used herein, a LAG3 antibody (anti-LAG3 antibody) generally refers to an antibody that binds to LAG3 with sufficient affinity such that the antibody is useful as a diagnostic and/or therapeutic agent in targeting LAG3 (e.g., inhibit LAG3-mediated immune suppression activities). In certain embodiments, the extent of binding of an anti-LAG3 antibody to an unrelated, non-betaglycan protein is less than about 10%, 9%, 8%, 7%, 6%, 5%, 4%, 3%, 2%, or less than about 1% of the binding of the antibody to LAG3 as measured, for example, by a radioimmunoassay (RIA), Biacore, or other protein-protein interaction or binding affinity assay. In certain embodiments, an anti-LAG3 antibody binds to an epitope of LAG3 that is conserved among LAG3 from different species. In certain preferred embodiments, an anti-LAG3 antibody binds to human LAG3.

In some embodiments, an immune checkpoint antagonist domain is an antibody, or antigen-binding domain thereof, that binds to TIM3. As used herein, a TIM3 antibody (anti-TIM3 antibody) generally refers to an antibody that binds to TIM3 with sufficient affinity such that the antibody is useful as a diagnostic and/or therapeutic agent in targeting TIM3 (e.g., inhibit TIM3-mediated immune suppression activities). In certain embodiments, the extent of binding of an anti-TIM3 antibody to an unrelated, non-betaglycan protein is less than about 10%, 9%, 8%, 7%, 6%, 5%, 4%, 3%, 2%, or less than about 1% of the binding of the antibody to TIM3 as measured, for example, by a radioimmunoassay (RIA), Biacore, or other protein-protein interaction or binding affinity assay. In certain embodiments, an anti-TIM3 antibody binds to an epitope of TIM3 that is conserved among TIM3 from different species. In certain preferred embodiments, an anti-TIM3 antibody binds to human TIM3.

In some embodiments, an immune checkpoint antagonist domain is an antibody, or antigen-binding domain thereof, that binds to LAIR1. As used herein, a LAIR1 antibody (anti-LAIR1 antibody) generally refers to an antibody that binds to LAIR1 with sufficient affinity such that the antibody is useful as a diagnostic and/or therapeutic agent in targeting LAIR1 (e.g., inhibit LAIR1-mediated immune suppression activities). In certain embodiments, the extent of binding of an anti-LAIR1 antibody to an unrelated, non-betaglycan protein is less than about 10%, 9%, 8%, 7%, 6%, 5%, 4%, 3%, 2%, or less than about 1% of the binding of the antibody to LAIR1 as measured, for example, by a radioimmunoassay (RIA), Biacore, or other protein-protein interaction or binding affinity assay. In certain embodiments, an anti-LAIR1 antibody binds to an epitope of LAIR1 that is conserved among LAIR1 from different species. In certain preferred embodiments, an anti-LAIR1 antibody binds to human LAIR1.

In some embodiments, an immune checkpoint antagonist domain is an antibody, or antigen-binding domain thereof, that binds to B7-DC. As used herein, a B7-DC antibody (anti-B7-DC antibody) generally refers to an antibody that binds to B7-DC with sufficient affinity such that the antibody is useful as a diagnostic and/or therapeutic agent in targeting B7-DC (e.g., inhibit B7-DC-mediated immune suppression activities). In certain embodiments, the extent of binding of an anti-B7-DC antibody to an unrelated, non-betaglycan protein is less than about 10%, 9%, 8%, 7%, 6%, 5%, 4%, 3%, 2%, or less than about 1% of the binding of the antibody to B7-DC as measured, for example, by a radioimmunoassay (RIA), Biacore, or other protein-protein interaction or binding affinity assay. In certain embodiments, an anti-B7-DC antibody binds to an epitope of B7-DC that is conserved among B7-DC from different species. In certain preferred embodiments, an anti-B7-DC antibody binds to human B7-DC.

In some embodiments, an immune checkpoint antagonist domain is an antibody, or antigen-binding domain thereof, that binds to HVEM. As used herein, a HVEM antibody (anti-HVEM antibody) generally refers to an antibody that binds to HVEM with sufficient affinity such that the antibody is useful as a diagnostic and/or therapeutic agent in targeting HVEM (e.g., inhibit HVEM-mediated immune suppression activities). In certain embodiments, the extent of binding of an anti-HVEM antibody to an unrelated, non-betaglycan protein is less than about 10%, 9%, 8%, 7%, 6%, 5%, 4%, 3%, 2%, or less than about 1% of the binding of the antibody to HVEM as measured, for example, by a radioimmunoassay (RIA), Biacore, or other protein-protein interaction or binding affinity assay. In certain embodiments, an anti-HVEM antibody binds to an epitope of HVEM that is conserved among HVEM from different species. In certain preferred embodiments, an anti-HVEM antibody binds to human HVEM.

In some embodiments, an immune checkpoint antagonist domain is an antibody, or antigen-binding domain thereof, that binds to TIM4. As used herein, a TIM4 antibody (anti-TIM4 antibody) generally refers to an antibody that binds to TIM4 with sufficient affinity such that the antibody is useful as a diagnostic and/or therapeutic agent in targeting TIM4 (e.g., inhibit TIM4-mediated immune suppression activities). In certain embodiments, the extent of binding of an anti-TIM4 antibody to an unrelated, non-betaglycan protein is less than about 10%, 9%, 8%, 7%, 6%, 5%, 4%, 3%, 2%, or less than about 1% of the binding of the antibody to TIM4 as measured, for example, by a radioimmunoassay (RIA), Biacore, or other protein-protein interaction or binding affinity assay. In certain embodiments, an anti-TIM4 antibody binds to an epitope of TIM4 that is conserved among TIM4 from different species. In certain preferred embodiments, an anti-TIM4 antibody binds to human TIM4.

In some embodiments, an immune checkpoint antagonist domain is an antibody, or antigen-binding domain thereof, that binds to B7-H3. As used herein, a B7-H3 antibody (anti-B7-H3 antibody) generally refers to an antibody that binds to B7-H3 with sufficient affinity such that the antibody is useful as a diagnostic and/or therapeutic agent in targeting B7-H3 (e.g., inhibit B7-H3-mediated immune suppression activities). In certain embodiments, the extent of binding of an anti-B7-H3 antibody to an unrelated, non-betaglycan protein is less than about 10%, 9%, 8%, 7%, 6%, 5%, 4%, 3%, 2%, or less than about 1% of the binding of the antibody to B7-H3 as measured, for example, by a radioimmunoassay (RIA), Biacore, or other protein-protein interaction or binding affinity assay. In certain embodiments, an anti-B7-H3 antibody binds to an epitope of B7-H3 that is conserved among B7-H3 from different species. In certain preferred embodiments, an anti-B7-H3 antibody binds to human B7-H3.

In some embodiments, an immune checkpoint antagonist domain is an antibody, or antigen-binding domain thereof, that binds to B7-H4. As used herein, a B7-H4 antibody (anti-B7-H4 antibody) generally refers to an antibody that binds to B7-H4 with sufficient affinity such that the antibody is useful as a diagnostic and/or therapeutic agent in targeting B7-H4 (e.g., inhibit B7-H4-mediated immune suppression activities). In certain embodiments, the extent of binding of an anti-B7-H4 antibody to an unrelated, non-betaglycan protein is less than about 10%, 9%, 8%, 7%, 6%, 5%, 4%, 3%, 2%, or less than about 1% of the binding of the antibody to B7-H4 as measured, for example, by a radioimmunoassay (RIA), Biacore, or other protein-protein interaction or binding affinity assay. In certain embodiments, an anti-B7-H4 antibody binds to an epitope of B7-H4 that is conserved among B7-H4 from different species. In certain preferred embodiments, an anti-B7-H4 antibody binds to human B7-H4.

The term antibody is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody fragment refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments. See, e.g., Hudson et al. (2003) Nat. Med. 9:129-134; Plückthun, in The Pharmacology of Monoclonal Antibodies, vol. 113, Rosenburg and Moore eds., (Springer-Verlag, New York), pp. 269-315 (1994); WO 93/16185; and U.S. Pat. Nos. 5,571,894, 5,587,458, and 5,869,046. Antibodies disclosed herein may be polyclonal antibodies or monoclonal antibodies. In certain embodiments, the antibodies of the present disclosure comprise a label attached thereto and able to be detected (e.g., the label can be a radioisotope, fluorescent compound, enzyme, or enzyme co-factor). In preferred embodiments, the antibodies of the present disclosure are isolated antibodies.

Diabodies are antibody fragments with two antigen-binding sites that may be bivalent or bispecific. See, e.g., EP 404,097; WO 1993/01161; Hudson et al. (2003) Nat. Med. 9:129-134 (2003); and Hollinger et al. (1993) Proc. Natl. Acad. Sci. USA 90: 6444-6448. Triabodies and tetrabodies are also described in Hudson et al. (2003) Nat. Med. 9:129-134.

Single-domain antibodies are antibody fragments comprising all or a portion of the heavy-chain variable domain or all or a portion of the light-chain variable domain of an antibody. In certain embodiments, a single-domain antibody is a human single-domain antibody. See, e.g., U.S. Pat. No. 6,248,516.

Antibody fragments can be made by various techniques, including but not limited to proteolytic digestion of an intact antibody as well as production by recombinant host cells (e.g., E. coli or phage), as described herein.

The antibodies herein may be of any class. The class of an antibody refers to the type of constant domain or constant region possessed by its heavy chain. There are five major classes of antibodies: IgA, IgD, IgE, IgG, and IgM, and several of these may be further divided into subclasses (isotypes), for example, IgG₁, IgG₂, IgG₃, IgG₄, IgA₁, and IgA₂. The heavy-chain constant domains that correspond to the different classes of immunoglobulins are called alpha, delta, epsilon, gamma, and mu.

In general, an antibody for use in the methods disclosed herein specifically binds to its target antigen, preferably with high binding affinity. Affinity may be expressed as a K_{D} value and reflects the intrinsic binding affinity (e.g., with minimized avidity effects). Typically, binding affinity is measured in vitro, whether in a cell-free or cell-associated setting. Any of a number of assays known in the art, including those disclosed herein, can be used to obtain binding affinity measurements including, for example, surface plasmon resonance (Biacore™ assay), radiolabeled antigen binding assay (RIA), and ELISA. In some embodiments, antibodies of the present disclosure bind to their target antigens [e.g., ActRIIB, ActRIIA, activin (e.g., activin A, activin B, activin C, activin E, activin AB, activin AC) TβRII, betaglycan, TGFβ (e.g., TGFβ1, TGFβ2, and TGFβ3), PD-1, PD-L1, CTLA4, BTLA, LAG3, TIM3, LAIR1, B7-DC, HVEM, TIM4, B7-H3, and B7-H4] with at least a K_{D} of 1×10⁻⁹ or stronger, 1×10⁻¹° or stronger, 1×10⁻¹¹ or stronger, 1×10⁻¹² or stronger, 1×10⁻¹³ or stronger, or 1×10⁻¹⁴ or stronger.

In certain embodiments, K_{D} is measured by RIA performed with the Fab version of an antibody of interest and its target antigen as described by the following assay. Solution binding affinity of Fabs for the antigen is measured by equilibrating Fab with a minimal concentration of radiolabeled antigen (e.g., ¹²⁵I-labeled) in the presence of a titration series of unlabeled antigen, then capturing bound antigen with an anti-Fab antibody-coated plate [see, e.g., Chen et al. (1999) J. Mol. Biol. 293:865-881]. To establish conditions for the assay, multi-well plates (e.g., MICROTITER® from Thermo Scientific) are coated (e.g., overnight) with a capturing anti-Fab antibody (e.g., from Cappel Labs) and subsequently blocked with bovine serum albumin, preferably at room temperature (e.g., approximately 23° C.). In a non-adsorbent plate, radiolabeled antigen are mixed with serial dilutions of a Fab of interest [e.g., consistent with assessment of the anti-VEGF antibody, Fab-12, in Presta et al., (1997) Cancer Res. 57:4593-4599]. The Fab of interest is then incubated, preferably overnight but the incubation may continue for a longer period (e.g., about 65 hours) to ensure that equilibrium is reached. Thereafter, the mixtures are transferred to the capture plate for incubation, preferably at room temperature for about one hour. The solution is then removed and the plate is washed times several times, preferably with polysorbate 20 and PBS mixture. When the plates have dried, scintillant (e.g., MICROSCINT® from Packard) is added, and the plates are counted on a gamma counter (e.g., TOPCOUNT® from Packard).

According to another embodiment, K_{D} is measured using surface plasmon resonance assays using, for example a BIACORE® 2000 or a BIACORE® 3000 (Biacore, Inc., Piscataway, N.J.) with immobilized antigen CM5 chips at about 10 response units (RU). Briefly, carboxymethylated dextran biosensor chips (CM5, Biacore, Inc.) are activated with N-ethyl-N′-(3-dimethylaminopropyl)-carbodiimide hydrochloride (EDC) and N-hydroxysuccinimide (NHS) according to the supplier's instructions. For example, an antigen can be diluted with 10 mM sodium acetate, pH 4.8, to 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20®) surfactant (PBST) at at a flow rate of approximately 25 μl/min. Association rates (kₒₙ) and dissociation rates (k_{off}) are calculated using, for example, a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ [see, e.g., Chen et al., (1999) J. Mol. Biol. 293:865-881]. If the on-rate exceeds, for example, 10⁶ M⁻¹ s⁻¹ by the surface plasmon resonance assay above, then the on-rate can be determined by using a fluorescent quenching technique that measures the increase or decrease in fluorescence emission intensity (e.g., excitation=295 nm; emission=340 nm, 16 nm band-pass) of a 20 nM anti-antigen antibody (Fab form) in PBS in the presence of increasing concentrations of antigen as measured in a spectrometer, such as a stop-flow equipped spectrophometer (Aviv Instruments) or a 8000-series SLM-AMINCO® spectrophotometer (ThermoSpectronic) with a stirred cuvette.

The nucleic acid and amino acid sequences of human ActRIIB, ActRIIA, activin (e.g., activin A, activin B, activin C, activin E, activin AB, and activin AC), TGFβ (e.g., TGFβ1, TGFβ2, and TGFβ3) TβRII, betaglycan, PD-1, PD-L1, CTLA4, BTLA, LAG3, TIM3, LAIR1, B7-DC, HVEM, TIM4, B7-H3, and B7-H4 are well known in the art and thus antibody antagonists for use in accordance with this disclosure may be routinely made by the skilled artisan based on the knowledge in the art and teachings provided herein.

In certain embodiments, an antibody provided herein is a chimeric antibody. A chimeric antibody refers to an antibody in which a portion of the heavy and/or light chain is derived from a particular source or species, while the remainder of the heavy and/or light chain is derived from a different source or species. Certain chimeric antibodies are described, for example, in U.S. Pat. No. 4,816,567; and Morrison et al., (1984) Proc. Natl. Acad. Sci. USA, 81:6851-6855. In some embodiments, a chimeric antibody comprises a non-human variable region (e.g., a variable region derived from a mouse, rat, hamster, rabbit, or non-human primate, such as a monkey) and a human constant region. In some embodiments, a chimeric antibody is a “class switched” antibody in which the class or subclass has been changed from that of the parent antibody. In general, chimeric antibodies include antigen-binding fragments thereof.

In certain embodiments, a chimeric antibody provided herein is a humanized antibody. A humanized antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

Humanized antibodies and methods of making them are reviewed, for example, in Almagro and Fransson (2008) Front. Biosci. 13:1619-1633 and are further described, for example, in Riechmann et al., (1988) Nature 332:323-329; Queen et al. (1989) Proc. Nat'l Acad. Sci. USA 86:10029-10033; U.S. Pat. Nos. 5,821,337, 7,527,791, 6,982,321, and 7,087,409; Kashmiri et al., (2005) Methods 36:25-34 [describing SDR (a-CDR) grafting]; Padlan, Mol. Immunol. (1991) 28:489-498 (describing “resurfacing”); Dall'Acqua et al. (2005) Methods 36:43-60 (describing “FR shuffling”); Osbourn et al. (2005) Methods 36:61-68; and Klimka et al. Br. J. Cancer (2000) 83:252-260 (describing the “guided selection” approach to FR shuffling).

Human framework regions that may be used for humanization include but are not limited to: framework regions selected using the “best-fit” method [see, e.g., Sims et al. (1993) J. Immunol. 151:2296]; framework regions derived from the consensus sequence of human antibodies of a particular subgroup of light-chain or heavy-chain variable regions [see, e.g., Carter et al. (1992) Proc. Natl. Acad. Sci. USA, 89:4285; and Presta et al. (1993) J. Immunol., 151:2623]; human mature (somatically mutated) framework regions or human germline framework regions [see, e.g., Almagro and Fransson (2008) Front. Biosci. 13:1619-1633]; and framework regions derived from screening FR libraries [see, e.g., Baca et cd., (1997) J. Biol. Chem. 272:10678-10684; and Rosok et cd., (1996) J. Biol. Chem. 271:22611-22618].

In certain embodiments, an antibody provided herein is a human antibody. Human antibodies can be produced using various techniques known in the art. Human antibodies are described generally in van Dijk and van de Winkel (2001) Curr. Opin. Pharmacol. 5: 368-74 and Lonberg (2008) Curr. Opin. Immunol. 20:450-459.

Human antibodies may be prepared by administering an immunogen [e.g., ActRIIB, ActRIIA, activin (e.g., activin A, activin B, activin C, activin E, activin AB, activin AC), TGFβ (e.g., TGFβ1, TGFβ2, and TGFβ3) TβRII, betaglycan, PD-1, PD-L1, CTLA4, BTLA, LAG3, TIM3, LAIR1, B7-DC, HVEM, TIM4, B7-H3, and B7-H4] to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. For a review of methods for obtaining human antibodies from transgenic animals, see, for example, Lonberg (2005) Nat. Biotechnol. 23:1117-1125; U.S. Pat. Nos. 6,075,181 and 6,150,584 (describing XENOMOUSE™ technology); U.S. Pat. No. 5,770,429 (describing HuMab® technology); U.S. Pat. No. 7,041,870 (describing K-M MOUSE® technology); and U.S. Patent Application Publication No. 2007/0061900 (describing VelociMouse® technology). Human variable regions from intact antibodies generated by such animals may be further modified, for example, by combining with a different human constant region.

Human antibodies provided herein can also be made by hybridoma-based methods. Human myeloma and mouse-human heteromyeloma cell lines for the production of human monoclonal antibodies have been described [see, e.g., Kozbor J. Immunol., (1984) 133: 3001; Brodeur et al. (1987) Monoclonal Antibody Production Techniques and Applications, pp. 51-63, Marcel Dekker, Inc., New York; and Boerner et al. (1991) J. Immunol., 147: 86]. Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, Xiandai Mianyixue (2006) 26(4):265-268 (2006) (describing human-human hybridomas). Human hybridoma technology (Trioma technology) is also described in Vollmers and Brandlein (2005) Histol. Histopathol., 20(3):927-937 (2005) and Vollmers and Brandlein (2005) Methods Find Exp.

Clin. Pharmacol., 27(3):185-91.

Human antibodies provided herein may also be generated by isolating Fv clone variable-domain sequences selected from human-derived phage display libraries. Such variable-domain sequences may then be combined with a desired human constant domain. Techniques for selecting human antibodies from antibody libraries are described herein.

For example, antibodies of the present disclosure may be isolated by screening combinatorial libraries for antibodies with the desired activity or activities. A variety of methods are known in the art for generating phage-display libraries and screening such libraries for antibodies possessing the desired binding characteristics. Such methods are reviewed, for example, in Hoogenboom et al. (2001) in Methods in Molecular Biology 178:1-37, O'Brien et al., ed., Human Press, Totowa, N.J. and further described, for example, in the McCafferty et al. (1991) Nature 348:552-554; Clackson et al., (1991) Nature 352: 624-628; Marks et al. (1992) J. Mol. Biol. 222:581-597; Marks and Bradbury (2003) in Methods in Molecular Biology 248:161-175, Lo, ed., Human Press, Totowa, N.J.; Sidhu et al. (2004) J. Mol. Biol. 338(2):299-310; Lee et al. (2004) J. Mol. Biol. 340(5):1073-1093; Fellouse (2004) Proc. Natl. Acad. Sci. USA 101(34):12467-12472; and Lee et al. (2004) J. Immunol. Methods 284(1-2): 119-132.

In certain phage display methods, repertoires of VH and VL genes are separately cloned by polymerase chain reaction (PCR) and recombined randomly in phage libraries, which can then be screened for antigen-binding phage as described in Winter et al. (1994) Ann. Rev. Immunol., 12: 433-455. Phage typically display antibody fragments, either as single-chain Fv (scFv) fragments or as Fab fragments. Libraries from immunized sources provide high-affinity antibodies to the immunogen [e.g., ActRIIB, ActRIIA, activin (e.g., activin A, activin B, activin C, activin E, activin AB, activin AC), TGFβ (e.g., TGFβ1, TGFβ2, and TGFβ3) TβRII, betaglycan, PD-1, PD-L1, CTLA4, BTLA, LAG3, TIM3, LAIR1, B7-DC, HVEM, TIM4, B7-H3, and B7-H4] without the requirement of constructing hybridomas. Alternatively, the naive repertoire can be cloned (e.g., from human) to provide a single source of antibodies directed against a wide range of non-self and also self-antigens without any immunization as described by Griffiths et al. (1993) EMBO J, 12: 725-734. Finally, naive libraries can also be made synthetically by cloning un-rearranged V-gene segments from stem cells and using PCR primers containing random sequence to encode the highly variable CDR3 regions and to accomplish rearrangement in vitro, as described by Hoogenboom and Winter (1992) J. Mol. Biol., 227: 381-388. Patent publications describing human antibody phage libraries include, for example: U.S. Pat. No. 5,750,373, and U.S. Patent Publication Nos. 2005/0079574, 2005/0119455, 2005/0266000, 2007/0117126, 2007/0160598, 2007/0237764, 2007/0292936, and 2009/0002360.

In certain embodiments, an antibody provided herein is a multispecific antibody, for example, a bispecific antibody. Multispecific antibodies (typically monoclonal antibodies) have binding specificities for at least two different epitopes (e.g., two, three, four, five, or six or more) on one or more (e.g., two, three, four, five, six or more) antigens.

Engineered antibodies with three or more functional antigen binding sites, including “octopus antibodies,” are also included herein (see, e.g., US 2006/0025576A1).

In certain embodiments, the antibodies disclosed herein are monoclonal antibodies. Monoclonal antibody refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present methods may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

For example, by using immunogens derived from activin A, anti-protein/anti-peptide antisera or monoclonal antibodies can be made by standard protocols [see, e.g., Antibodies: A Laboratory Manual (1988) ed. by Harlow and Lane, Cold Spring Harbor Press]. A mammal, such as a mouse, hamster, or rabbit can be immunized with an immunogenic form of the activin A polypeptide, an antigenic fragment which is capable of eliciting an antibody response, or a fusion protein. Techniques for conferring immunogenicity on a protein or peptide include conjugation to carriers or other techniques well known in the art. An immunogenic portion of an activin A polypeptide can be administered in the presence of adjuvant. The progress of immunization can be monitored by detection of antibody titers in plasma or serum. Standard ELISA or other immunoassays can be used with the immunogen as antigen to assess the levels of antibody production and/or level of binding affinity.

Following immunization of an animal with an antigenic preparation of activin A, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique [see, e.g., Kohler and Milstein (1975) Nature, 256: 495-497], the human B cell hybridoma technique [see, e.g., Kozbar et al. (1983) Immunology Today, 4:72], and the EBV-hybridoma technique to produce human monoclonal antibodies [Cole et al. (1985) Monoclonal Antibodies and Cancer Therapy, Alan R. Liss, Inc. pp. 77-96]. Hybridoma cells can be screened immunochemically for production of antibodies specifically reactive with a activin A polypeptide, and monoclonal antibodies isolated from a culture comprising such hybridoma cells.

In certain embodiments, one or more amino acid modifications may be introduced into the Fc region of an antibody provided herein thereby generating an Fc-region variant. The Fc-region variant may comprise a human Fc-region sequence (e.g., a human IgG₁, IgG₂, IgG₃ or IgG₄ Fc region) comprising an amino acid modification (e.g., a substitution, deletion, and/or addition) at one or more amino acid positions.

For example, the present disclosure contemplates an antibody variant that possesses some but not all effector functions, which make it a desirable candidate for applications in which the half-life of the antibody in vivo is important yet for which certain effector functions [e.g., complement-dependent cytotoxicity (CDC) and antibody-dependent cellular cytotoxicity (ADCC)] are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγR1, FcγRII and FcγRIII. FcR expression on hematopoietic cells is summarized in, for example, Ravetch and Kinet (1991) Annu. Rev. Immunol. 9:457-492. Non-limiting examples of in vitro assays to assess ADCC activity of a molecule of interest are described in U.S. Pat. No. 5,500,362; Hellstrom, I. et al. (1986) Proc. Nat'l Acad. Sci. USA 83:7059-7063; Hellstrom, I et al. (1985) Proc. Nat'l Acad. Sci. USA 82:1499-1502; U.S. Pat. No. 5,821,337; and Bruggemann, M. et al. (1987) J. Exp. Med. 166:1351-1361. Alternatively, non-radioactive assay methods may be employed (e.g., ACTI™, non-radioactive cytotoxicity assay for flow cytometry; CellTechnology, Inc. Mountain View, Calif.; and CytoTox 96® non-radioactive cytotoxicity assay, Promega, Madison, Wis.). Useful effector cells for such assays include peripheral blood mononuclear cells (PBMC) and natural killer (NK) cells. Alternatively, or additionally, ADCC activity of the molecule of interest may be assessed in vivo, for example, in an animal model such as that disclosed in Clynes et al. (1998) Proc. Nat'l Acad. Sci. USA 95:652-656. C1q binding assays may also be carried out to confirm that the antibody is unable to bind C1q and hence lacks CDC activity [see, e.g., C1q and C3c binding ELISA in WO 2006/029879 and WO 2005/100402]. To assess complement activation, a CDC assay may be performed [see, e.g., Gazzano-Santoro et al. (1996) J. Immunol. Methods 202:163; Cragg, M. S. et al. (2003) Blood 101:1045-1052; and Cragg, M. S, and M. J. Glennie (2004) Blood 103:2738-2743]. FcRn binding and in vivo clearance/half-life determinations can also be performed using methods known in the art [see, e.g., Petkova, S. B. et al. (2006) Int. Immunol. 18(12):1759-1769].

Antibodies of the present disclosure with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, it may be desirable to create cysteine-engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy-chain Fc region. Cysteine engineered antibodies may be generated as described, for example, in U.S. Pat. No. 7,521,541.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore™ binding assay, Biacore AB, Uppsala, Sweden), sandwich assays (e.g., the paramagnetic bead system of IGEN International, Inc., Gaithersburg, Md.), western blots, immunoprecipitation assays, and immunohistochemistry.

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into, and/or substitutions of residues within, the amino acid sequences of the antibody and/or binding polypeptide. Any combination of deletion, insertion, and substitution can be made to arrive at the final construct, provided that the final construct possesses the desired characteristics, e.g., target-binding (e.g., ActRIIB, ActRIIA, activin (e.g., activin A, activin B, activin C, activin E, activin AB, and activin AC), TGFβ (TGFβ1, TGFβ2, and TGFβ3) TβRII, betaglycan, PD-1, PD-L1, CTLA4, BTLA, LAG3, TIM3, LAIR1, B7-DC, HVEM, TIM4, B7-H3, and B7-H4 binding).

Alterations (e.g., substitutions) may be made in HVRs, for example, to improve antibody affinity. Such alterations may be made in HVR “hotspots,” i.e., residues encoded by codons that undergo mutation at high frequency during the somatic maturation process (see, e.g., Chowdhury (2008) Methods Mol. Biol. 207:179-196 (2008)), and/or SDRs (a-CDRs), with the resulting variant VH or VL being tested for binding affinity. Affinity maturation by constructing and reselecting from secondary libraries has been described in the art [see, e.g., Hoogenboom et al., in Methods in Molecular Biology 178:1-37, O'Brien et al., ed., Human Press, Totowa, N.J., (2001)]. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind to the antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two, or three amino acid substitutions.

A useful method for identification of residues or regions of the antibody and/or the binding polypeptide that may be targeted for mutagenesis is called “alanine scanning mutagenesis”, as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or binding polypeptide with antigen is affected. Further substitutions may be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues may be targeted or eliminated as candidates for substitution. Variants may be screened to determine whether they contain the desired properties.

Amino-acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include fusion of the N- or C-terminus of the antibody to an enzyme (e.g., for ADEPT) or a polypeptide which increases the serum half-life of the antibody.

In certain embodiments, an antibody and/or binding polypeptide provided herein may be further modified to contain additional non-proteinaceous moieties that are known in the art and readily available. The moieties suitable for derivatization of the antibody and/or binding polypeptide include but are not limited to water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions.

### 5. Small Molecule Antagonists

In other aspects, the present disclosure relates to an activin antagonist, a TGFβ antagonist, and/or an immune checkpoint antagonist that is small molecule, or combination of small molecules. In particular, the disclosure relates in part to using such small molecules in combination with bi- or tri-functional fusion proteins comprising two or more of an activin antagonist domain, a TGFβ antagonist domain, and an immune checkpoint antagonist domain treat or prevent one or more diseases or conditions described herein (e.g., cancer, tumors, pre-neoplastic disorders, hyperproliferative disorders, and dysplastic disorders).

In certain aspects, the small molecule is an activin antagonist. In some embodiments, the small molecule inhibits activin A, activin B, activin C, activin E, activin AB, and/or activin AC. In some embodiments, the small molecule inhibits activin A, but does not inhibit or sustainably inhibit activin B. In some embodiments, the small molecule inhibits activin B, but does not inhibit or sustainably inhibit activin A. In some embodiments, the small molecule inhibits activin A and activin B. In some embodiments, the small molecule inhibits ActRIIA, but does not inhibit or sustainably inhibit ActRIIB. In some embodiments, the small molecule inhibits ActRIIB, but does not inhibit or sustainably inhibit ActRIIA. In some embodiments, the small molecule inhibits ActRIIA and ActRIIB.

In certain aspects, the small molecule is a TGFβ antagonist. In some embodiments, the small molecule inhibits at least TGFβ (e.g., TGFβ1, TGFβ2, and/or TGFβ3). In some embodiments, the small molecule inhibits TGFβ1 and TGFβ3, but does not inhibit or sustainably inhibit TGFβ2. In some embodiments, the small molecule inhibits TGFβ1, TGFβ2, and TGFβ3. In some embodiments, the small molecule inhibits TβRII. In some embodiments, the small molecule inhibits betaglycan.

In certain aspects, the small molecule is an immune checkpoint antagonist. In some embodiments, the small molecule inhibits PD-1. In some embodiments, the small molecule inhibits PD-L1. In some embodiments, the small molecule inhibits CTLA4. In some embodiments, the small molecule inhibits BTLA. In some embodiments, the small molecule inhibits LAG3. In some embodiments, the small molecule inhibits TIM3. In some embodiments, the small molecule inhibits LAIR1. In some embodiments, the small molecule inhibits B7-DC. In some embodiments, the small molecule inhibits HVEM. In some embodiments, the small molecule inhibits TIM4. In some embodiments, the small molecule inhibits B7-H3. In some embodiments, the small molecule inhibits B7-H4.

Small molecule antagonists can be direct or indirect inhibitors. For example, an indirect small molecule antagonist, or combination of small molecule antagonists, may inhibit the expression (e.g., transcription, translation, cellular secretion, or combinations thereof) of at least one or more proteins [e.g., activin A, activin B, activin C, activin E, activin AB, activin AC), TGFβ1, TGFβ2, TGFβ 3 TβRII, betaglycan, PD-1, PD-L1, CTLA4, BTLA, LAG3, TIM3, LAIR1, B7-DC, HVEM, TIM4, B7-H3, and B7-H4]. Alternatively, a direct small molecule antagonist, or combination of small molecule antagonists, may directly bind to, for example, one or more proteins [e.g., activin A, activin B, activin C, activin E, activin AB, activin AC), TGFβ1, TGFβ2, TGFβ 3 TβRII, betaglycan, PD-1, PD-L1, CTLA4, BTLA, LAG3, TIM3, LAIR1, B7-DC, HVEM, TIM4, B7-H3, and B7-H4] and thereby inhibit activity of said one or more proteins. Combinations of one or more indirect and one or more direct small molecule antagonists may be used in accordance with the methods disclosed herein.

Binding organic small molecule antagonists of the present disclosure may be identified and chemically synthesized using known methodology (see, e.g., PCT Publication Nos. WO 00/00823 and WO 00/39585). In general, small molecule antagonists of the disclosure are usually less than about 2000 daltons in size, alternatively less than about 1500, 750, 500, 250 or 200 daltons in size, wherein such organic small molecules that are capable of binding, preferably specifically, to a polypeptide as described herein. Such small molecule antagonists may be identified without undue experimentation using well-known techniques. In this regard, it is noted that techniques for screening organic small molecule libraries for molecules that are capable of binding to a polypeptide target are well-known in the art (see, e.g., international patent publication Nos. WO00/00823 and WO00/39585).

Binding organic small molecules of the present disclosure may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkones, alkynes, diols, amino alcohols, oxazolidines, oxazolines, thiazolidines, thiazolines, enamines, sulfonamides, epoxides, aziridines, isocyanates, sulfonyl chlorides, diazo compounds, and acid chlorides.

### 6. Polynucleotide Antagonists

In other aspects, the present disclosure relates to an activin antagonist, a TGFβ antagonist, and/or an immune checkpoint antagonist that is a polynucleotide, or combination of polynucleotides. In particular, the disclosure relates in part to using such polynucleotides in combination with bi- or tri-functional fusion proteins comprising two or more of an activin antagonist domain, a TGFβ antagonist domain, and an immune checkpoint antagonist domain treat or prevent one or more diseases or conditions described herein (e.g., cancer, tumors, pre-neoplastic disorders, hyperproliferative disorders, and dysplastic disorders).

In certain aspects, the polynucleotides is an activin antagonist. In some embodiments, the polynucleotide inhibits at least activin (e.g., activin A, activin B, activin C, activin E, activin AB, and/or activin AC). In some embodiments, the polynucleotide inhibits activin A, but does not inhibit or sustainably inhibit activin B. In some embodiments, the polynucleotide inhibits activin B, but does not inhibit or sustainably inhibit activin A. In some embodiments, the polynucleotide inhibits activin A and activin B. In some embodiments, the polynucleotide inhibits ActRIIA, but does not inhibit or sustainably inhibit ActRIIB. In some embodiments, the polynucleotide inhibits ActRIIB, but does not inhibit or sustainably inhibit ActRIIA. In some embodiments, the polynucleotide inhibits ActRIIA and ActRIIB.

In certain aspects, the polynucleotide is a TGFβ antagonist. In some embodiments, the polynucleotide inhibits at least TGFβ (e.g., TGFβ1, TGFβ2, and/or TGFβ 3). In some embodiments, the polynucleotide inhibits TGFβ1 and TGFβ3, but does not inhibit or sustainably inhibit TGFβ2. In some embodiments, the polynucleotide inhibits TGFβ1, TGFβ2, and TGFβ3. In some embodiments, the polynucleotide inhibits TβRII. In some embodiments, the polynucleotide inhibits betaglycan.

In certain aspects, the polynucleotide is an immune checkpoint antagonist. In some embodiments, the polynucleotide inhibits PD-1. In some embodiments, the polynucleotide inhibits PD-L1. In some embodiments, the polynucleotide inhibits CTLA4. In some embodiments, the polynucleotide inhibits BTLA. In some embodiments, the polynucleotide inhibits LAG3. In some embodiments, the polynucleotide inhibits TIM3. In some embodiments, the polynucleotide inhibits LAIR1. In some embodiments, the polynucleotide inhibits B7-DC. In some embodiments, the polynucleotide inhibits HVEM. In some embodiments, the polynucleotide inhibits TIM4. In some embodiments, the polynucleotide inhibits B7-H3. In some embodiments, the polynucleotide inhibits B7-H4.

The polynucleotide antagonists of the present disclosure may be an antisense nucleic acid, an RNAi molecule [e.g., small interfering RNA (siRNA), small-hairpin RNA (shRNA), microRNA (miRNA)], an aptamer and/or a ribozyme. The nucleic acid and amino acid sequences of human activin A, activin B, activin C, activin E, activin AB, activin AC), TGFβ L TGFβ2, TGFβ 3 TβRII, betaglycan, PD-1, PD-L1, CTLA4, BTLA, LAG3, TIM3, LAIR1, B7-DC, HVEM, TIM4, B7-H3, and B7-H4, are known in the art and thus polynucleotide antagonists for use in accordance with methods of the present disclosure may be routinely made by the skilled artisan based on the knowledge in the art and teachings provided herein.

For example, antisense technology can be used to control gene expression through antisense DNA or RNA, or through triple-helix formation. Antisense techniques are discussed, for example, in Okano (1991) J. Neurochem. 56:560; Oligodeoxynucleotides as Antisense Inhibitors of Gene Expression, CRC Press, Boca Raton, Fla. (1988). Triple helix formation is discussed in, for instance, Cooney et al. (1988) Science 241:456; and Dervan et al., (1991) Science 251:1300. The methods are based on binding of a polynucleotide to a complementary DNA or RNA. In some embodiments, the antisense nucleic acids comprise a single-stranded RNA or DNA sequence that is complementary to at least a portion of an RNA transcript of a desired gene. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double-stranded antisense nucleic acids of a gene disclosed herein, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with an RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, for example, the 5′-untranslated sequence up to and including the AUG initiation codon, should work most efficiently at inhibiting translation. However, sequences complementary to the 3′-untranslated sequences of mRNAs have been shown to be effective at inhibiting translation of mRNAs as well [see, e.g., Wagner, R., (1994) Nature 372:333-335]. Thus, oligonucleotides complementary to either the 5′- or 3′-untranslated, noncoding regions of a gene of the disclosure, could be used in an antisense approach to inhibit translation of an endogenous mRNA. Polynucleotides complementary to the 5′-untranslated region of the mRNA should include the complement of the AUG start codon. Antisense polynucleotides complementary to mRNA coding regions are less efficient inhibitors of translation but could be used in accordance with the methods of the present disclosure. Whether designed to hybridize to the 5′-untranslated, 3′-untranslated, or coding regions of an mRNA of the disclosure, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides.

In one embodiment, the antisense nucleic acid of the present disclosure is produced intracellularly by transcription from an exogenous sequence. For example, a vector or a portion thereof, is transcribed, producing an antisense nucleic acid (RNA) of a gene of the disclosure. Such a vector would contain a sequence encoding the desired antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding desired genes of the instant disclosure, or fragments thereof, can be by any promoter known in the art to act in vertebrate, preferably human cells. Such promoters can be inducible or constitutive. Such promoters include, but are not limited to, the SV40 early promoter region [see, e.g., Benoist and Chambon (1981) Nature 29:304-310], the promoter contained in the 3′ long terminal repeat of Rous sarcoma virus [see, e.g., Yamamoto et al. (1980) Cell 22:787-797], the herpes thymidine promoter [see, e.g., Wagner et al. (1981) Proc. Natl. Acad. Sci. U.S.A. 78:1441-1445], and the regulatory sequences of the metallothionein gene [see, e.g., Brinster, et al. (1982) Nature 296:39-42].

In some embodiments, the polynucleotide antagonists are interfering RNA or RNAi molecules that target the expression of one or more genes. RNAi refers to the expression of an RNA which interferes with the expression of the targeted mRNA. Specifically, RNAi silences a targeted gene via interacting with the specific mRNA through a siRNA (small interfering RNA). The ds RNA complex is then targeted for degradation by the cell. An siRNA molecule is a double-stranded RNA duplex of 10 to 50 nucleotides in length, which interferes with the expression of a target gene which is sufficiently complementary (e.g. at least 80% identity to the gene). In some embodiments, the siRNA molecule comprises a nucleotide sequence that is at least 85, 90, 95, 96, 97, 98, 99, or 100% identical to the nucleotide sequence of the target gene.

Additional RNAi molecules include short-hairpin RNA (shRNA); also short-interfering hairpin and microRNA (miRNA). The shRNA molecule contains sense and antisense sequences from a target gene connected by a loop. The shRNA is transported from the nucleus into the cytoplasm, and it is degraded along with the mRNA. Pol III or U6 promoters can be used to express RNAs for RNAi. Paddison et al. [Genes & Dev. (2002) 16:948-958, 2002] have used small RNA molecules folded into hairpins as a means to effect RNAi. Accordingly, such short hairpin RNA (shRNA) molecules are also advantageously used in the methods described herein. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the double-stranded RNA (dsRNA) products of the DICER RNase and, in any event, have the same capacity for inhibiting expression of a specific gene. The shRNA can be expressed from a lentiviral vector. An miRNA is a single-stranded RNA of about 10 to 70 nucleotides in length that are initially transcribed as pre-miRNA characterized by a “stem-loop” structure and which are subsequently processed into mature miRNA after further processing through the RISC.

Molecules that mediate RNAi, including without limitation siRNA, can be produced in vitro by chemical synthesis (Hohjoh, FEBS Lett 521:195-199, 2002), hydrolysis of dsRNA (Yang et al., Proc Natl Acad Sci USA 99:9942-9947, 2002), by in vitro transcription with T7 RNA polymerase (Donzeet et al., Nucleic Acids Res 30:e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947, 2002).

According to another aspect, the disclosure provides polynucleotide antagonists including but not limited to, a decoy DNA, a double-stranded DNA, a single-stranded DNA, a complexed DNA, an encapsulated DNA, a viral DNA, a plasmid DNA, a naked RNA, an encapsulated RNA, a viral RNA, a double-stranded RNA, a molecule capable of generating RNA interference, or combinations thereof.

In some embodiments, the polynucleotide antagonists of the disclosure are aptamers. Aptamers are nucleic acid molecules, including double-stranded DNA and single-stranded RNA molecules, which bind to and form tertiary structures that specifically bind to a target molecule, such as a activin A, activin B, activin C, activin E, activin AB, activin AC), TGFβ1, TGFβ2, TGFβ3 TβRII, betaglycan, PD-1, PD-L1, CTLA4, BTLA, LAG3, TIM3, LAIR1, B7-DC, HVEM, TIM4, B7-H3, and B7-H4 polypeptide. The generation and therapeutic use of aptamers are well established in the art. See, e.g., U.S. Pat. No. 5,475,096. Additional information on aptamers can be found in U.S. Patent Application Publication No. 20060148748. Nucleic acid aptamers are selected using methods known in the art, for example via the Systematic Evolution of Ligands by Exponential Enrichment (SELEX) process. SELEX is a method for the in vitro evolution of nucleic acid molecules with highly specific binding to target molecules as described in, e.g., U.S. Pat. Nos. 5,475,096, 5,580,737, 5,567,588, 5,707,796, 5,763,177, 6,011,577, and 6,699,843. Another screening method to identify aptamers is described in U.S. Pat. No. 5,270,163. The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets. The SELEX method involves selection from a mixture of candidate oligonucleotides and step-wise iterations of binding, partitioning and amplification, using the same general selection scheme, to achieve desired binding affinity and selectivity. Starting from a mixture of nucleic acids, which can comprise a segment of randomized sequence, the SELEX method includes steps of contacting the mixture with the target under conditions favorable for binding; partitioning unbound nucleic acids from those nucleic acids which have bound specifically to target molecules; dissociating the nucleic acid-target complexes; amplifying the nucleic acids dissociated from the nucleic acid-target complexes to yield a ligand enriched mixture of nucleic acids. The steps of binding, partitioning, dissociating and amplifying are repeated through as many cycles as desired to yield highly specific high affinity nucleic acid ligands to the target molecule.

Typically, such binding molecules are separately administered to the animal [see, e.g., O'Connor (1991) J. Neurochem. 56:560], but such binding molecules can also be expressed in vivo from polynucleotides taken up by a host cell and expressed in vivo [see, e.g., Oligodeoxynucleotides as Antisense Inhibitors of Gene Expression, CRC Press, Boca Raton, Fla. (1988)].

### 8. Exemplary Therapeutic Uses

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treatment”, “treating”, “alleviation” and the like are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

As used herein, “combination”, “in combination with”, “conjoint administration” and the like refers to any form of administration such that the second therapy is still effective in the body (e.g., the two compounds are simultaneously effective in the patient, which may include synergistic effects of the two compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, an individual who receives such treatment can benefit from a combined effect of different therapies. One or more bi- or tri-functional fusion proteins of the disclosure can be administered concurrently with, prior to, or subsequent to, one or more other additional agents or supportive therapies. In general, each therapeutic agent will be administered at a dose and/or on a time schedule determined for that particular agent. The particular combination to employ in a regimen will take into account compatibility of the antagonist of the present disclosure with the therapy and/or the desired therapeutic effect to be achieved.

In part, the data presented herein demonstrates that activin antagonists and TGFβ antagonists may be used alone or in combination to treat cancer. In particular, it was shown that treatment with an ActRIIA polypeptide, an ActRIIB polypeptide, or a pan-specific TGFβ antibody, separately, decreased tumor burden and increased survival time a cancer model. Moreover, it was shown that an activin antagonist in combination with a TGFβ antagonist may be used to synergistically increase antitumor activity compared to the effects observed with either agent alone. While not wishing to be bound by any particular theory, it is believed that such activin and TGFβ antagonist, alone or in combination, may be particularly useful in treating cancer when used in combination with an immune checkpoint antagonist (e.g., an antibody, or antigen-binding fragment thereof, that binds and inhibits one or more of (e.g., PD-1, PD-L1, CTLA4, BTLA, LAG3, TIM3, LAIR1, B7-DC, HVEM, TIM4, B7-H3, and/or B7-H4). Accordingly, the disclosure provides, in part, bi- and tri-functional fusion proteins comprising two or more domains selected from an activin antagonist domain, a TGFβ antagonist domain, and an immune checkpoint antagonist domain. The disclosure further provides, for example, methods of using such bi- and tri-functional fusion proteins to treat cancer, a tumor, a pre-neoplastic disorder, a hyperproliferative disorder, or a dysplastic disorder. Optionally, such methods further comprise administering to the patient an additional active agent or supportive therapy for treating the cancer, tumor, pre-neoplastic disorder, hyperproliferative disorder, or dysplastic disorder. As with other known immuno-oncology agents, the ability of such bi- and tri-functional fusion proteins to potentiate an immune response in a patient may have broader therapeutic implications outside the cancer field. For example, it has been proposed that immune potentiating agents may be useful in treating a wide variety of infectious diseases, particularly pathogenic agents which promote immunosuppression and/or immune exhaustion. Also, such immune potentiating agents may be useful in boosting the immunization efficacy of vaccines (e.g., infectious disease and cancer vaccines).

In general, “tumors” refers to benign and malignant cancers, as well as dormant tumors. In general, “cancer” refers to primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Metastasis can be local or distant. Metastases are most often detected through the sole or combined use of magnetic resonance imaging (MRI) scans, computed tomography (CT) scans, blood and platelet counts, liver function studies, chest X-rays, bone scans in addition to the monitoring of specific symptoms, and combinations thereof.

Bi- and tri-functional fusion proteins of the disclosure may be used to treat various forms of cancer, tumors, pre-neoplastic disorders, hyperproliferative disorders, and dysplastic disorders including, but not limited to, cancer of the bladder, breast, colon, kidney, liver, lung, ovary, cervix, pancreas, rectum, prostate, stomach, epidermis, and brain. Examples of cancers that may be treated by bi- and tri-functional fusion proteins of the disclosure include, but are not limited to, a hematopoietic tumor of lymphoid or myeloid lineage tumor of mesenchymal origin such as a fibrosarcoma or rhabdomyosarcoma, melanoma, intraocular melanoma, nonmelanoma skin cancer, teratocarci-noma, neuroblastoma, glioma, brain stem glioma, visual pathway and hypothalamic glioma, oligodendroglioma, adenocarcinoma, papillary adenocarcinomas, cystadenocarcinoma, carcinoma, non-small lung cell carcinoma, hepatoma, hepatocellular carcinoma, endometrial cancer or uterine carcinoma, salivary gland carcinoma, differentiated thyroid carcinoma, carcinoma of the lung, penile carcinoma, adrenocortical carcinoma, endocrine pancreas islet cell carcinoma, colon carcinoma, squamous cell carcinoma, basal cell carcinoma, sweat gland carcinoma, sebaceous gland carcinoma, papillary carcinoma, medullary carcinoma, bronchogenic carcinoma, renal cell carcinoma, anal carcinoma, bile duct carcinoma, choriocarcinoma, embryonal carcinoma, epithelial carcinoma, lymphoma, adult Hodgkin's lymphoma, adult non-Hodgkin's lymphoma, AIDS-related lymphoma, central nervous system lymphoma, cutaneous T-cell lymphoma, T-Cell lymphoma, seminoma, glioblastoma, glioblastoma multiforme, sarcoma, Ewing sarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, soft tissue sarcoma, Kaposi's sarcoma, osteo/malignant fibrous sarcoma, osteosarcoma/malignant fibrous histiocytoma, sarcoidosis sarcoma, uterine sarcoma, lymphangioendotheliosarcoma, leukemia, acute lymphoblastic leukemia, acute lymphocytic leukemia, acute myeloid leukemia, chronic lymphocytic leukemia, chronic myelogenous leukemia, hairy cell leukemia, myelogenous leukemia, myeloid leukemia, myeloblastic leukemia, promyelocytic leukemia, myelomonocytic leukemia, monocytic leukemia, a erythroleukemia, chronic myelocytic leukemia, leukemia myeloma, multiple myeloma, lymphoid malignancies, squamous cell cancer, epithelial squamous cell cancer, squamous cancer of the peritoneum, squamous neck cancer, metastatic squamous neck cancer, metastatic squamous neck cancer, occult metastatic squamous neck cancer, Wilms tumor, astrocytomas, lung cancer, small-cell lung cancer, non-small cell lung cancer, hepatocellular cancer, gastric or stomach cancer, gastrointestinal cancer, gastrointestinal carcinoid tumor, pancreatic cancer, exocrine pancreatic cancer, islet cell pancreatic cancer, cervical cancer, cervical dysplasia, ovarian cancer, ovarian epithelial cancer, ovarian germ cell tumor, ovarian low malignant potential tumor, liver cancer, neuroendocrine tumors, medullary thyroid cancer, parathyroid cancer, breast cancer, colon cancer, rectal cancer, kidney or renal cancer, prostate cancer, vulvar cancer, head-and-neck cancer, AIDS-related malignancies, anal cancer, astrocytoma, cerebellar astrocytoma, cerebral astrocytoma, bile duct cancer, extrahepatic bile duct cancer, bone cancer, fibrous dysplasia of bone, brain tumors, extracranial germ cell tumors, extragonadal germ cell tumor, germ cell tumors, Hodgkin's disease, medulloblastoma, pineal tumors, pinealoma, supratentorial neuroectodermal tumors, ependymoma, epithelial cancer, epithelial dysplasia, mucoepithelial dysplasia, esophageal cancer, esophageal dysplasia, eye cancer, Gaucher's disease, gallbladder cancer, gestational TROPhoblastic tumor, TROPhoblastic tumors, hypergammaglobulinemia, hypopharyngeal cancer, intestinal cancers, intestinal polyps or adenomas, small intestine cancer, large intestine cancer, laryngeal cancer, lip or oral cavity cancer, lymphoproliferative disorders, macroglobulinemia, Waldenstrom's macroglobulinemia, mesothelioma, malignant thymoma, thymoma, metastatic occult plasma cell neoplasm, myelodysplastic syndrome, myeloproliferative disorders, nasal cavity or paranasal sinus cancer, nasopharyngeal cancer, oropharyngeal cancer, paraproteinemias, penile cancer, pheochromocytoma, pituitary tumor, retinoblastoma, salivary gland cancer, Sezary syndrome, skin cancer, testicular cancer, urethral cancer, uterine cancer, vaginal cancer, anhidrotic ectodermal dysplasia, anterofacial dysplasia, asphyxiating thoracic dysplasia, atriodigital dysplasia, bronchopulmonary dysplasia, cerebral dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, faciodigitogenital dysplasia, familial fibrous dysplasia of jaws, familial white folded dysplasia, fibromuscular dysplasia, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, ventriculoradial dysplasia, benign dysproliferative disorders (e.g., benign tumors, fibrocystic conditions, tissue hypertrophy, and), leukoplakia, keratoses, Bowen's disease, Farmer's skin, solar cheilitis, solar keratosis, heavy chain disease, synovioma, craniopharyngioma, emangioblastoma, acoustic neuroma, and meningioma.

In certain aspects, bi- and tri-functional fusion proteins of the disclosure may be used in combination with one or more additional active agents or supportive therapies to treat various forms of cancer, tumors, pre-neoplastic disorders, hyperproliferative disorders, and dysplastic disorders. For example, additional therapeutic agents to treat one or more of cancer, tumors, pre-neoplastic disorders, hyperproliferative disorders, and dysplastic disorders included, but are not limited to cytotoxic agents, anti-angiogenic agents, pro-apoptotic agents, immunomodulator agents, antibiotics, hormones, hormone antagonists, chemokines, prodrugs, toxins, enzymes or other active agents. Additional active agents of use may possess a pharmaceutical property selected from, for example: antimitotic, anti-kinase, alkylating, antimetabolite, antibiotic, alkaloid, anti-angiogenic, pro-apoptotic agents, and combinations thereof.

In some embodiments, additional active agents or supportive therapies include, for example, one or more of fluorouracil, afatinib, aplidin, azaribine, anastrozole, anthracyclines, axitinib, aminoglutethimide, amsacrine, AVL-101, AVL-291, bendamustine, bleomycin, buserelin, bortezomib, bosutinib, bicalutamide, bryostatin-1, busulfan, capecitabine, calicheamycin, camptothecin, carboplatin, 10-hydroxycamptothecin, carmustine, celebrex, chlorambucil, cisplatin (CDDP), Cox-2 inhibitors, irinotecan (CPT-11), SN-38, cladribine, camptothecans, crizotinib, colchicine, cyclophosphamide, cytarabine, cyproterone, clodronate, dacarbazine, dasatinib, dienestrol, dinaciclib, docetaxel, dactinomycin, daunorubicin, diethylstilbestrol, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, filgrastim, fingolimod, floxuridine (FUdR), fluoxymesterone, 3′,5′-O-dioleoyl-FudR (FUdR-dO), fludrocortisone, fludarabine, flutamide, goserelin, farnesyl-protein transferase inhibitors, flavopiridol, fostamatinib, ganetespib, GDC-0834, GS-1101, gefitinib, gemcitabine, hydroxyurea, ibrutinib, idarubicin, levamisole, idelalisib, ifosfamide, imatinib, letrozole, asparaginase, leuprolide, lapatinib, lenolidamide, leucovorin, ironotecan, LFM-A13, lomustine, mechlorethamine, melphalan, mercaptopurine, 6-mercaptopurine, megestrol, methotrexate, mitoxantrone, nilutamide, mithramycin, mitomycin, nocodazole, octreotide, mitotane, navelbine, neratinib, nilotinib, nitrosurea, olaparib, plicomycin, procarbazine, paclitaxel, oxaliplatin, PCI-32765, pentostatin, plicamycin, PSI-341, raloxifene, semustine, sorafenib, streptozocin, SU11248, sunitinib, tamoxifen, porfimer, temozolomide, mesna, temazolomide (an aqueous form of DTIC), transplatinum, thalidomide, thioguanine, raltitrexed, thiotepa, teniposide, topotecan, uracil mustard, vatalanib, vinorelbine, vinblastine, rituximab, pamidronate, vincristine, vinca alkaloids, ZD1839, ricin, abrin, alpha toxin, saporin, ribonucleases (e.g., onconase) DNase I, Staphylococcal enterotoxin-A, pokeweed antiviral protein, gelonin, diphtheria toxin, Pseudomonas exotoxin, Pseudomonas endotoxin, RANTES, MCAF, MIP 1-alpha, MIP 1-beta, IP-10, angiostatin, baculostatin, canstatin, maspin, anti-VEGF antibodies, anti-P1GF peptides and antibodies, anti-vascular growth factor antibodies, anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-beta, thrombospondin, 2-methoxyoestradiol, proliferin-related protein, carboxiamidotriazole, CM101, Marimastat, pentosan polysulphate, angiopoietin-2, interferon-alpha, herbimycin A, PNU145156E, 16K prolactin fragment, Linomide (roquinimex), thalidomide, pentoxifylline, genistein, TNP-470, endostatin, paclitaxel, accutin, angiostatin, cidofovir, vincristine, bleomycin, AGM-1470, platelet factor 4, ALK1 polypeptides (e.g., dalantercept), minocycline, a cytokine, a stem cell growth factor, a lymphotoxin, a hematopoietic factor, a colony stimulating factor (CSF), an interferon (IFN), erythropoietin, thrombopoietin, lymphotoxins, tumor necrosis factor (TNF), hematopoietic factors, interleukin (IL), colony stimulating factor, granulocyte-colony stimulating factor (G-CSF), granulocyte macrophage-colony stimulating factor (GM-CSF), interferons, interferons-alpha, -beta or -lamda, stem cell growth factor, Si factor, human growth hormone, N-methionyl human growth hormone, bovine growth hormone, parathyroid hormone, thyroxine, insulin, proinsulin, relaxin, prorelaxin, glycoprotein hormones, follicle stimulating hormone (FSH), thyroid stimulating hormone (TSH), luteinizing hormone (LH), hepatic growth factor, prostaglandin, fibroblast growth factor, prolactin, placental lactogen, OB protein, tumor necrosis factor-alpha and/or -beta, mullerian-inhibiting substance, mouse gonadotropin-associated peptide, vascular endothelial growth factor, integrin, thrombopoietin (TPO), nerve growth factors such as NGF-beta, platelet-growth factor, insulin-like growth factor-I and/or —II, erythropoietin (EPO), osteoinductive factors, interleukins (ILs) (e.g., IL-1, IL-1alpha, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3), angiostatin, thrombospondin, endostatin, tumor necrosis factor, LT, an alkylating agent, a nitrosourea, an anti-metabolite, a topoisomerase inhibitor, a mitotic inhibitor, an anthracycline, a corticosteroid hormone, a sex hormone, a targeted anti-tumor compound, imatinib (Gleevec), gefitinib (Iressa), erlotinib (Tarceva), rituximab (Rituxan), bevacizumab (Avastin), busulfan, cisplatin, carboplatin, chlorambucil, cyclophosphamide, ifosfamide, dacarbazine (DTIC), mechlorethamine (nitrogen mustard), melphalan, temozolomide, 5-fluorouracil, capecitabine, 6-mercaptopurine, methotrexate, gemcitabine, cytarabine (ara-C), fludarabine, pemetrexed, topotecan, irinotecan, etoposide (VP-16), teniposide, daunorubicin, doxorubicin (Adriamycin), epirubicin, idarubicin, or mitoxantrone.

In some embodiments, additional active agents or supportive therapies include, for example, one or more radionuclides. Radionuclides that may be used in accordance with the methods of the disclosure include, but are not limited to, ¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²¹¹Pb, and ²²⁷Th. In some embodiments, the therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. In general, maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³I, ¹⁰³Ru, ¹⁰⁵Ru, ¹⁰⁷Hg, ²⁰³Hg, ¹²¹mTe, ¹²²mTe, ¹²⁵mTe, ¹⁶⁵Tm, ¹⁶⁷Tm, ⁷⁷Br, ¹¹³mIn, ⁹⁵Ru, ⁹⁷Ru, ¹⁶⁸Tm, ¹⁹⁷Pr, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵⁵e, ²⁰¹Tl, ²²⁵Ac, ⁷⁶Br, and ¹⁶⁹Yb.

In some embodiments, additional active agents or supportive therapies include, for example, one or more photoactive agents or dyes. Fluorescent compositions, such as fluorochrome, and other chromogens, or dyes, such as porphyrins sensitive to visible light, may be used detect and to treat lesions by directing the suitable light to the lesion. In therapy, this has been termed photoradiation, phototherapy, or photodynamic therapy. See Joni et al. (eds.), (Libreria Progetto 1985); van den Bergh, Chem. Britain (1986), 22:430. Moreover, monoclonal antibodies may be coupled with photoactivated dyes for achieving phototherapy. See Mew et al., J. Immunol. (1983), 130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Some cancers/tumors can escape immune surveillance by co-opting certain immune-checkpoint pathways, particularly in T cells that are specific for tumor antigens (Pardoll, 2012, Nature Reviews Cancer 12:252-264). Studies with checkpoint inhibitor antibodies for cancer therapy have been successful in treating cancers previously thought to be resistant to cancer treatment (see, e.g., Ott & Bhardwaj, 2013, Frontiers in Immunology 4:346; Menzies & Long, 2013, Ther Adv Med Oncol 5:278-85; Pardoll, 2012, Nature Reviews Cancer 12:252-64; Mavilio & Lugli). In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264). Because such inhibitors act primarily by regulating the immune response to diseased cells, tissues or pathogens, they may be used in combination with other therapeutic modalities, ADCs, and/or interferons to enhance the anti-tumor effect of such agents.

Anti-PD1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD1 antibodies include pembrolizumab (MK-3475, Merck), nivolumab (BMS-936558, Bristol-Myers Squibb), AMP-224 (GlaxoSmithKline), AMP-514 (GlaxoSmithKline), pidilizumab (CT-011, Curetech Ltd.), PDR001 (Novartis), cemiplimab (Regeneron and Sanofi). Anti-PD1 antibodies are commercially available, for example from ABCAM (AB137132), Biolegend (EH12.2H7, RMP1-14) and Affymetrix Ebioscience (J105, J116, MIH4).

Anti-PD-L1 antibodies have been used for the treatment of urothelial carcinoma, non-small cell lung cancer, metastatic merkel-cell carcinoma, and gastric cancer. Exemplary anti-PD-L1 antibodies include atezolizumab (Roche Genetech), avelumab (Merck Serono and Pfizer), and durvalumab (AstraZeneca).

Anti-CTL4A antibodies have been used in clinical trials for treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). Exemplary anti-CTLA4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

Although checkpoint inhibitor against CTLA4, PD1 and PD-L1 are the most clinically advanced, other potential checkpoint antigens are known and may be used as the target of therapeutic inhibitors including, for example, LAG3, B7-H3, B7-H4, TIM3, BTLA, LAIR1, B7-DC, HVEM, and TIM4 (e.g., Pardoll, 2012, Nature Reviews Cancer 12:252-264).

In certain aspects, bi- or tri-functional fusion proteins of the disclosure may comprise, or be used in combination with, one or more checkpoint inhibitors. Exemplary checkpoint inhibitors that may comprise a portion of the bi- or tri-functional fusion proteins of the disclosure, or may be used in combination with the bi- or tri-functional fusion proteins of the disclosure, include inhibitors of one or more of CTLA4, PD1, PD-L1, LAG3, B7-H3, B7-H4, TIM3, BTLA, LAIR1, B7-DC, HVEM, and TIM4. In some embodiments, the checkpoint inhibitor that may comprise a portion of the bi- or tri-functional fusion proteins of the disclosure, or may be used in combination with the bi- or tri-functional fusion proteins of the disclosure, include inhibitors of one or more of CTLA4, PD1, and PD-L1. In some embodiments, a CTLA4 inhibitor (e.g., a CTLA4 antibody) comprises a portion of the bi- or tri-functional fusion proteins of the disclosure, or may be used in combination with the bi- or tri-functional proteins of the disclosure. In some embodiments, a PD1 inhibitor (e.g., a PD1 antibody) comprises a portion of the bi- or tri-functional fusion proteins of the disclosure, or may be used in combination with the bi- or tri-functional proteins of the disclosure. In some embodiments, a PD-L1 inhibitor (e.g., a PD-L1 antibody) comprises a portion of the bi- or tri-functional fusion proteins of the disclosure, or may be used in combination with the bi- or tri-functional fusion proteins of the disclosure.

In certain aspects, bi- and tri-functional fusion proteins of the disclosure may be more effective in treating various forms of cancer, tumors, pre-neoplastic disorders, hyperproliferative disorders, and/or dysplastic disorders when combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogeneic tumor cells. These cellular vaccines have been shown to have increased effectiveness when the tumor cells are transduced to express GM-CSF (Dranoff et al. (1993) Proc. Natl. Acad. Sci U.S.A. 90: 3539-43). Therefore, in some embodiments, one or more bi- and tri-functional proteins may be combined with an immunogenic agent, such as cancerous cells, purified tumor antigens (including recombinant proteins, peptides, and carbohydrate molecules), cells, and cells transfected with genes encoding immune stimulating cytokines (He et al (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF.

In other aspects, methods of the disclosure directed to treating patients that have been exposed to particular toxins or pathogens. Accordingly, the disclosure further provides methods of treating or preventing an infectious disease (e.g., viral, bacterial or parasitic infection) in a subject comprising administering to an subject in need thereof an therapeutically effective amount of one or more bi- or tri-functional fusion proteins of the disclosure, optionally further comprising administering one or more additional supportive therapies and/or active agents for treating the infectious disease.

In some embodiments, bi- or tri-functional fusion proteins of the disclosure may be used to treat or prevent infection by one or more viruses, bacteria, or parasites selected from Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxyiridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses), chickonpox, common cold, viral bronchitis, cytomegalovirus infection, Colorado tick fever, Dengue fever, Ebola haemorrhagic fever, epidemic parotitis, “hand, foot and mouth” disease, hepatitis, herpes simplex, herpes zoster, HPV, Influenza (Flu), Lassa fever, measles, Marburg haemorrhagic fever, infectious mononucleosis, mumps, poliomyelitis, progressive multifocal leukoncephalopathy, rabies, rubella, SARS, smallpox, viral encephalitis, viral gastroenteritis, viral meningitis, viral pneumonia, West Nile disease, Yellow fever, Helicobacter pyloris, Borelia burgdorferi, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracia, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, and Actinomyces israelli, anthrax, bacterial adult respiratory distress syndrome, bacterial meningitis, brucellosis, campylobacteriosis, cat scratch disease, bronchitis, cholera, diphtheria, typhus, gonorrhea, legionellosis, leprosy (Hansen's Disease), leptospirosis, listeriosis, lyme disease, melioidosis, MRSA infection, mycobacterial infection, meningitis, nocardiosis, nephritis, glomerulonephritis, periodontal disease, pertussis (Whooping Cough), plague, pneumococcal pneumonia, psittacosis, Q fever, Rocky Mountain Spotted Fever (RMSF), salmonellosis, scarlet dever, shigellosis, syphilis, septic shock, haemodynamic shock, sepsis syndrome, tetanus, trachoma, tuberculosis, tularemia, typhoid Fever, Cryptococcus neoformans, Histoplasma capsulatum, Coccidioides immitis, Blastomyces dermatitidis, and Candida albicans, aspergillosis; thrush, cryptococcosis, blastomycosis, coccidioidomycosis, ahistoplasmosis, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, Nippostrongylus brasiliensis, African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, and Trypanosomiasis.

In some embodiments, the disclosure provides methods of treating an infectious disease by administering to a patient in need thereof an effective amount of a bi- or tri-functional fusion protein of the disclosure in combination with a second therapeutic agent to treat the pathogen, for example, an antibiotic, antifungal agent, antiviral agent, or anti-parasite drug.

### 9. Pharmaceutical Compositions

The agents described herein (e.g., bi- and tri-functional fusion proteins) may be formulated into pharmaceutical compositions. Pharmaceutical compositions for use in accordance with the present disclosure may be formulated in conventional manner using one or more physiologically acceptable carriers or excipients. Such formulations will generally be substantially pyrogen-free, in compliance with most regulatory requirements.

In certain embodiments, the therapeutic method of the disclosure includes administering the composition systemically, or locally as an implant or device. When administered, the therapeutic composition for use in this disclosure is in a pyrogen-free, physiologically acceptable form. Therapeutically useful agents other than the bi- or tri-functional fusion protein of the disclosure which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more bi- or tri-functional fusion proteins of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., bi- and tri-functional proteins) to a target tissue site, providing a structure for the developing tissue and optimally capable of being resorbed into the body. For example, the matrix may provide slow release of the bi- and tri-functional proteins of the disclosure. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above-mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the invention (e.g., bi- and tri-functional proteins). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling.

In certain embodiments, the present invention also provides gene therapy for the in vivo production of bi- and tri-functional proteins of the disclosure. Such therapy would achieve its therapeutic effect by introduction of the bi- or tri-functional polynucleotide sequences into cells or tissues having the disorders as listed above. Delivery bi- or tri-functional polynucleotide sequences can be achieved using a recombinant expression vector such as a chimeric virus or a colloidal dispersion system. Preferred for therapeutic delivery of bi- or tri-functional polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the bi- or tri-functional polynucleotide. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for bi- or tri-functional polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### EXEMPLIFICATION

The invention now being generally described, it will be more readily understood by reference to the following examples, which are included merely for purposes of illustration of certain embodiments and embodiments of the present invention, and are not intended to limit the invention.

### Example 1. Generation of TβRII Receptor Fusion Protein Variants TβRII ECD Variants

TβRII fusion proteins comprising a soluble extracellular portion of human TβRII and a human Fc portion were generated. For each fusion protein, a TβRII amino acid sequence having the amino acid sequence of SEQ ID NO: 18 was fused to an IgG Fc portion having the amino acid sequence of SEQ ID NO: 49 by means of one of several different linkers. Each of the fusion proteins also included a TPA leader sequence having the amino acid sequence of SEQ ID NO: 23 (below).

Tissue plasminogen activator (TPA):
(SEQ ID NO: 23)
MDAMKRGLCCVLLLCGAVEVSP

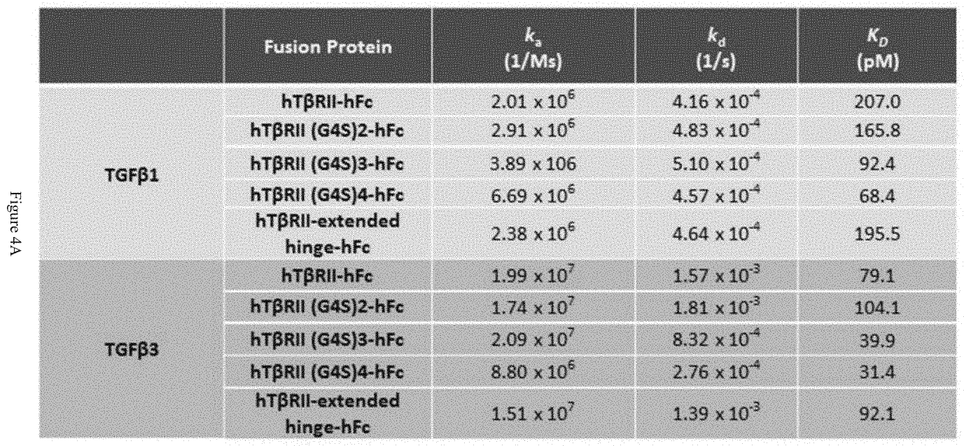

An illustration summary of several of the constructs designed is provided as FIG. 3. A table detailing the sequences for the different constructs tested in the Exemplification section is provided below:

| | Construct | |
|---|---|---|
| Construct | Amino Acid | |
| Name | Sequence | Linker Sequence |
| hTβRII-hFc | SEQ ID NO: 9 | TGGG (SEQ ID NO: 3) |
| hTβRII (G4S)2- | SEQ ID NO: 15 | TGGGGSGGGGS |
| hFc | | (SEQ ID NO: 4) |
| hTβRII (G4S)3- | SEQ ID NO: 11 | TGGGGSGGGGSGGGGS |
| hFc | | (SEQ ID NO: 5) |
| hTβRII (G4S)4- | SEQ ID NO: 13 | TGGGGSGGGGSGGGGSGG |
| hFc | | GGS (SEQ ID NO: 6) |
| hTβRII | SEQ ID NO: 17 | TGGGPKSCDK |
| extended | | (SEQ ID NO: 7) |
| hinge-hFc | | |
| hTβRII (G4S)5- | SEQ ID NO: 44 | TGGGGSGGGGSGGGGSGG |
| hFc | | GGSGGGGS (SEQ ID |
| | | NO: 25) |
| hTβRII (G4S)6- | SEQ ID NO: 45 | TGGGGSGGGGSGGGGSGG |
| hFc | | GGSGGGGSGGGGS (SEQ |
| | | ID NO: 26) |

The amino acid sequences for the construct components and each of the constructs, along with the nucleic acid sequence used to express these constructs, are provided below.

| TβRII Portion: Amino Acid Sequence | |
|---|---|
| (SEQ ID NO: 18) | |
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PD |
| Fe Portion: Amino Acid Sequence | |
| (SEQ ID NO: 49) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| hTβRII-hFc: Nucleic Acid Sequence | |
| (SEQ ID NO: 8) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA ACTCACACAT GCCCACCGTG CCCAGCACCT |
| 601 | GAACTCCTGG GGGGACCGTC AGTCTTCCTC TTCCCCCCAA AACCCAAGGA |
| 651 | CACCCTCATG ATCTCCCGGA CCCCTGAGGT CACATGCGTG GTGGTGGACG |
| 701 | TGAGCCACGA AGACCCTGAG GTCAAGTTCA ACTGGTACGT GGACGGCGTG |
| 751 | GAGGTGCATA ATGCCAAGAC AAAGCCGCGG GAGGAGCAGT ACAACAGCAC |
| 801 | GTACCGTGTG GTCAGCGTCC TCACCGTCCT GCACCAGGAC TGGCTGAATG |
| 851 | GCAAGGAGTA CAAGTGCAAG GTCTCCAACA AAGCCCTCCC AGCCCCCATC |
| 901 | GAGAAAACCA TCTCCAAAGC CAAAGGGCAG CCCCGAGAAC CACAGGTGTA |
| 951 | CACCCTGCCC CCATCCCGGG AGGAGATGAC CAAGAACCAG GTCAGCCTGA |
| 1001 | CCTGCCTGGT CAAAGGCTTC TATCCCAGCG ACATCGCCGT GGAGTGGGAG |
| 1051 | AGCAATGGGC AGCCGGAGAA CAACTACAAG ACCACGCCTC CCGTGCTGGA |
| 1101 | CTCCGACGGC TCCTTCTTCC TCTATAGCAA GCTCACCGTG GACAAGAGCA |
| 1151 | GGTGGCAGCA GGGGAACGTC TTCTCATGCT CCGTGATGCA TGAGGCTCTG |
| 1201 | CACAACCACT ACACGCAGAA GAGCCTCTCC CTGTCTCCGG GTAAATGA |
| hTβRII-hFc: Amino Acid Sequence | |
| (SEQ ID NO: 9) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG THTCPPCPAP |
| 201 | ELLGGPSVFL FPPKPKDTLM ISRTPEVTCV VVDVSHEDPE VKFNWYVDGV |
| 251 | EVHNAKTKPR EEQYNSTYRV VSVLTVLHQD WLNGKEYKCK VSNKALPAPI |
| 301 | EKTISKAKGQ PREPQVYTLP PSREEMTKNQ VSLTCLVKGF YPSDIAVEWE |
| 351 | SNGQPENNYK TTPPVLDSDG SFFLYSKLTV DKSRWQQGNV FSCSVMHEAL |
| 401 | HNHYTQKSLS LSPGK |

For animal experiments below, a variant form of SEQ ID NO: 9 was used wherein the human Fc domain was replaced by a mouse IgG₁ Fc domain. The variant is designated a hTβRII-mFc

| hTβRII (G4S)3-hFc: Nucleic Acid Sequence | |
|---|---|
| (SEQ ID NO: 10) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA GGAAGTGGTG GAGGTGGTTC TGGAGGTGGT |
| 60h | GGAAGTACTC ACACATGCCC ACCGTGCCCA GCACCTGAAC TCCTGGGGGG |
| 65h | ACCGTCAGTC TTCCTCTTCC CCCCAAAACC CAAGGACACC CTCATGATCT |
| 701 | CCCGGACCCC TGAGGTCACA TGCGTGGTGG TGGACGTGAG CCACGAAGAC |
| 751 | CCTGAGGTCA AGTTCAACTG GTACGTGGAC GGCGTGGAGG TGCATAATGC |
| 301 | CAAGACAAAG CCGCGGGAGG AGCAGTACAA CAGCACGTAC CGTGTGGTCA |
| 351 | GCGTCCTCAC CGTCCTGCAC CAGGACTGGC TGAATGGCAA GGAGTACAAG |
| 901 | TGCAAGGTCT CCAACAAAGC CCTCCCAGCC CCCATCGAGA AAACCATCTC |
| 951 | CAAAGCCAAA GGGCAGCCCC GAGAACCACA GGTGTACACC CTGCCCCCAT |
| 1001 | CCCGGGAGGA GATGACCAAG AACCAGGTCA GCCTGACCTG CCTGGTCAAA |
| 1051 | GGCTTCTATC CCAGCGACAT CGCCGTGGAG TGGGAGAGCA ATGGGCAGCC |
| 1101 | GGAGAACAAC TACAAGACCA CGCCTCCCGT GCTGGACTCC GACGGCTCCT |
| 1151 | TCTTCCTCTA TAGCAAGCTC ACCGTGGACA AGAGCAGGTG GCAGCAGGGG |
| 1201 | AACGTCTTCT CATGCTCCGT GATGCATGAG GCTCTGCACA ACCACTACAC |
| 1251 | GCAGAAGAGC CTCTCCCTGT CTCCGGGTAA ATGA |
| hTβRII (G4S)3-hFc: Amino Acid Sequence | |
| (SEQ ID NO: 11) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSTHTCPPCP APELLGGPSV FLFPPKPKDT LMISRTPEVT CVVVDVSHED |
| 251 | PEVKFNWYVD GVEVHNAKTK PREEQYNSTY RVVSVLTVLH QDWLNGKEYK |
| 301 | CKVSNKALPA PIEKTISKAK GQPREPQVYT LPPSREEMTK NQVSLTCLVK |
| 351 | GFYPSDIAVE WESNGQPENN YKTTPPVLDS DGSFFLYSKL TVDKSRWQQG |
| 401 | NVFSCSVMHE ALHNHYTQKS LSLSPGK |
| hTβRII (G4S)4-hFc: Nucleic Acid Sequence | |
| (SEQ ID NO: 12) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA GGTTCTGGAG GTGGAGGAAG TGGTGGAGGT |
| 601 | GGTTCTGGAG GTGGTGGAAG TACTCACACA TGCCCACCGT GCCCAGCACC |
| 651 | TGAACTCCTG GGGGGACCGT CAGTCTTCCT CTTCCCCCCA AAACCCAAGG |
| 701 | ACACCCTCAT GATCTCCCGG ACCCCTGAGG TCACATGCGT GGTGGTGGAC |
| 751 | GTGAGCCACG AAGACCCTGA GGTCAAGTTC AACTGGTACG TGGACGGCGT |
| 801 | GGAGGTGCAT AATGCCAAGA CAAAGCCGCG GGAGGAGCAG TACAACAGCA |
| 851 | CGTACCGTGT GGTCAGCGTC CTCACCGTCC TGCACCAGGA CTGGCTGAAT |
| 901 | GGCAAGGAGT ACAAGTGCAA GGTCTCCAAC AAAGCCCTCC CAGCCCCCAT |
| 951 | CGAGAAAACC ATCTCCAAAG CCAAAGGGCA GCCCCGAGAA CCACAGGTGT |
| 1001 | ACACCCTGCC CCCATCCCGG GAGGAGATGA CCAAGAACCA GGTCAGCCTG |
| 1051 | ACCTGCCTGG TCAAAGGCTT CTATCCCAGC GACATCGCCG TGGAGTGGGA |
| 1101 | GAGCAATGGG CAGCCGGAGA ACAACTACAA GACCACGCCT CCCGTGCTGG |
| 1151 | ACTCCGACGG CTCCTTCTTC CTCTATAGCA AGCTCACCGT GGACAAGAGC |
| 1201 | AGGTGGCAGC AGGGGAACGT CTTCTCATGC TCCGTGATGC ATGAGGCTCT |
| 1251 | GCACAACCAC TACACGCAGA AGAGCCTCTC CCTGTCTCCG GGTAAATGA |
| hTβRII (G4S)4-hFc: Amino Acid Sequence | |
| (SEQ ID NO: 13) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSTHT CPPCPAPELL GGPSVFLFPP KPKDTLMISR TPEVTCVVVD |
| 251 | VSHEDPEVKF NWYVDGVEVH NAKTKPREEQ YNSTYRVVSV LTVLHQDWLN |
| 301 | GKEYKCKVSN KALPAPIEKT ISKAKGQPRE PQVYTLPPSR EEMTKNQVSL |
| 351 | TCLVKGFYPS DIAVEWESNG QPENNYKTTP PVLDSDGSFF LYSKLTVDKS |
| 401 | RWQQGNVFSC SVMHEALHNH YTQKSLSLSP GK |
| hTβRII (G4S)4-hFc: Amino Acid Sequence lacking leader sequence | |
| (SEQ ID NO: 94) | |
| 1 | GATIPPHVQK SDVEMEAQKD EIICPSCNRT AHPLRHINND MIVTDNNGAV |
| 51 | KFPQLCKFCD VRFSTCDNQK SCMSNCSITS ICEKPQEVCV AVWRKNDENI |
| 101 | TLETVCHDPK LPYHDFILED AASPKCIMKE KKKPGETFFM CSCSSDECND |
| 151 | NIIFSEEYNT SNPDTGGGGS GGGGSGGGGS GGGGSTHTCP PCPAPELLGG |
| 201 | PSVFLFPPKP KDTLMISRTP EVTCVVVDVS HEDPEVKFNW YVDGVEVHNA |
| 251 | KTKPREEQYN STYRVVSVLT VLHQDWLNGK EYKCKVSNKA LPAPIEKTIS |
| 301 | KAKGQPREPQ VYTLPPSREE MTKNQVSLTC LVKGFYPSDI AVEWESNGQP |
| 351 | ENNYKTTPPV LDSDGSFFLY SKLTVDKSRW QQGNVFSCSV MHEALHNHYT |
| 401 | QKSLSLSPGK |
| hTβRII (G4S)4-hFc: Amino Acid Sequence lacking leader sequence | |
| and lacking glycine prior to hTβRII portion | |
| (SEQ ID NO: 95) | |
| 1 | ATIPPHVQKS DVEMEAQKDE IICPSCNRTA HPLRHINNDM IVTDNNGAVK |
| 51 | FPQLCKFCDV RFSTCDNQKS CMSNCSITSI CEKPQEVCVA VWRKNDENIT |
| 101 | LETVCHDPKL PYHDFILEDA ASPKCIMKEK KKPGETFFMC SCSSDECNDN |
| 151 | IIFSEEYNTS NPDTGGGGSG GGGSGGGGSG GGGSTHTCPP CPAPELLGGP |
| 201 | SVFLFPPKPK DTLMISRTPE VTCVVVDVSH EDPEVKFNWY VDGVEVHNAK |
| 251 | TKPREEQYNS TYRVVSVLTV LHQDWLNGKE YKCKVSNKAL PAPIEKTISK |
| 301 | AKGQPREPQV YTLPPSREEM TKNQVSLTCL VKGFYPSDIA VEWESNGQPE |
| 351 | NNYKTTPPVL DSDGSFFLYS KLTVDKSRWQ QGNVFSCSVM HEALHNHYTQ |
| 401 | KSLSLSPGK |
| hTβRII (G4S)4-hFc: Amino Acid Sequence lacking leader sequence | |
| and lacking glycine and alanine prior to hTβRII portion | |
| (SEQ ID NO: 96) | |
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PDTGGGGSGG GGSGGGGSGG GGSTHTCPPC RAPELLGGPS |
| 201 | VFLFPPKPKD TLMISRTPEV TCVVVDVSHE DPEVKFNWYV DGVEVHNAKT |
| 251 | KPREEQYNST YRVVSVLTVL HQDWLNGKEY KCKVSNKALP APIEKTISKA |
| 301 | KGQPREPQVY TLPPSREEMT KNQVSLTCLV KGFYPSDIAV EWESNGQPEN |
| 351 | NYKTTPPVLD SDGSFFLYSK LTVDKSRWQQ GNVFSCSVMH EALHNHYTQK |
| 401 | SLSLSPGK |
| hTβRII (G4S)4-hFc: Amino Acid Sequence lacking leader sequence | |
| and lacking glycine, alanine, and threonine prior to hTβRII | |
| portion | |
| (SEQ ID NO: 97) | |
| 1 | IPPHVQKSDV EMEAQKDEII CPSCNRTAHP LRHINNDMIV TDNNGAVKFP |
| 51 | QLCKFCDVRF STCDNQKSCM SNCSITSICE KPQEVCVAVW RKNDENITLE |
| 101 | TVCHDPKLPY HDFILEDAAS PKCIMKEKKK PGETFFMCSC SSDECNDNII |
| 151 | FSEEYNTSNP DTGGGGSGGG GSGGGGSGGG GSTHTCPPCP APELLGGPSV |
| 201 | FLFPPKPKDT LMISRTPEVT CVVVDVSHED PEVKFNWYVD GVEVHNAKTK |
| 251 | PREEQYNSTY RVVSVLTVLH QDWLNGKEYK CKVSNKALPA PIEKTISKAK |
| 301 | GQPREPQVYT LPPSREEMTK NQVSLTCLVK GFYPSDIAVE WESNGQPENN |
| 351 | YKTTPPVLDS DGSFFLYSKL TVDKSRWQQG NVFSCSVMHE ALHNHYTQKS |
| 401 | LSLSPGK |
| hTβRII (G4S)4-hFc: Amino Acid Sequence lacking leader sequence | |
| and lacking glycine, alanine, threonine, and isoleucine prior | |
| to hTβRII portion | |
| (SEQ ID NO: 98) | |
| 1 | PPHVQKSDVE MEAQKDEIIC PSCNRTAHPL RHINNDMIVT DNNGAVKFPQ |
| 51 | LCKFCDVRFS TCDNQKSCMS NCSITSICEK PQEVCVAVWR KNDENITLET |
| 101 | VCHDPKLPYH DFILEDAASP KCIMKEKKKP GETFFMCSCS SDECNDNIIF |
| 151 | SEEYNTSNPD TGGGGSGGGG SGGGGSGGGG STHTCPPCPA PELLGGPSVF |
| 201 | LFPPKPKDTL MISRTPEVTC VVVDVSHEDP EVKFNWYVDG VEVHNAKTKP |
| 251 | REEQYNSTYR VVSVLTVLHQ DWLNGKEYKC KVSNKALPAP IEKTISKAKG |
| 301 | QPREPQVYTL PPSREEMTKN QVSLTCLVKG FYPSDIAVEW ESNGQPENNY |
| 351 | KTTPPVLDSD GSFFLYSKLT VDKSRWQQGN VFSCSVMHEA LHNHYTQKSL |
| 401 | SLSPGK |
| hTβRII (G4S)4-hFc: Amino Acid Sequence lacking leader sequence | |
| and lacking glycine, alanine, threonine, isoleucine, and | |
| proline prior to hTβRII portion | |
| (SEQ ID NO: 99) | |
| 1 | PHVQKSDVEM EAQKDEIICP SCNRTAHPLR HINNDMIVTD NNGAVKFPQL |
| 51 | CKFCDVRFST CDNQKSCMSN CSITSICEKP QEVCVAVWRK NDENITLETV |
| 101 | CHDPKLPYHD FILEDAASPK CIMKEKKKPG ETFFMCSCSS DECNDNIIFS |
| 151 | EEYNTSNPDT GGGGSGGGGS GGGGSGGGGS THTCPPCPAP ELLGGPSVFL |
| 201 | FPPKPKDTLM ISRTPEVTCV VVDVSHEDPE VKFNWYVDGV EVHNAKTKPR |
| 251 | EEQYNSTYRV VSVLTVLHQD WLNGKEYKCK VSNKALPAPI EKTISKAKGQ |
| 301 | PREPQVYTLP PSREEMTKNQ VSLTCLVKGF YPSDIAVEWE SNGQPENNYK |
| 351 | TTPPVLDSDG SFFLYSKLTV DKSRWQQGNV FSCSVMHEAL HNHYTQKSLS |
| 401 | LSPGK |
| hTβRII (G4S)4-hFc: Amino Acid Sequence lacking leader sequence | |
| and lacking glycine, alanine, threonine, isoleucine, proline, | |
| and proline prior to hTβRII portion | |
| (SEQ ID NO: 100) | |
| 1 | HVQKSDVEME AQKDEIICPS CNRTAHPLRH INNDMIVTDN NGAVKFPQLC |
| 51 | KFCDVRFSTC DNQKSCMSNC SITSICEKPQ EVCVAVWRKN DENITLETVC |
| 101 | HDPKLPYHDF ILEDAASPKC IMKEKKKPGE TFFMCSCSSD ECNDNIIFSE |
| 151 | EYNTSNPDTG GGGSGGGGSG GGGSGGGGST HTCPPCPAPE LLGGPSVFLF |
| 201 | PPKPKDTLMI SRTPEVTCVV VDVSHEDPEV KFNWYVDGVE VHNAKTKPRE |
| 251 | EQYNSTYRVV SVLTVLHQDW LNGKEYKCKV SNKALPAPIE KTISKAKGQP |
| 301 | REPQVYTLPP SREEMTKNQV SLTCLVKGFY PSDIAVEWES NGQPENNYKT |
| 351 | TPPVLDSDGS FFLYSKLTVD KSRWQQGNVF SCSVMHEALH NHYTQKSLSL |
| 401 | SPGK |
| hTβRII (G4S)2-hFc: Nucleic Acid Sequence | |
| (SEQ ID NO: 14) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGAGGT GGTTCTGGAG GTGGTGGAAG TACTCACACA |
| 601 | TGCCCACCGT GCCCAGCACC TGAACTCCTG GGGGGACCGT CAGTCTTCCT |
| 651 | CTTCCCCCCA AAACCCAAGG ACACCCTCAT GATCTCCCGG ACCCCTGAGG |
| 701 | TCACATGCGT GGTGGTGGAC GTGAGCCACG AAGACCCTGA GGTCAAGTTC |
| 751 | AACTGGTACG TGGACGGCGT GGAGGTGCAT AATGCCAAGA CAAAGCCGCG |
| 601 | GGAGGAGCAG TACAACAGCA CGTACCGTGT GGTCAGCGTC CTCACCGTCC |
| 351 | TGCACCAGGA CTGGCTGAAT GGCAAGGAGT ACAAGTGCAA GGTCTCCAAC |
| 901 | AAAGCCCTCC CAGCCCCCAT CGAGAAAACC ATCTCCAAAG CCAAAGGGCA |
| 951 | GCCCCGAGAA CCACAGGTGT ACACCCTGCC CCCATCCCGG GAGGAGATGA |
| 1001 | CCAAGAACCA GGTCAGCCTG ACCTGCCTGG TCAAAGGCTT CTATCCCAGC |
| 1051 | GACATCGCCG TGGAGTGGGA GAGCAATGGG CAGCCGGAGA ACAACTACAA |
| 1101 | GACCACGCCT CCCGTGCTGG ACTCCGACGG CTCCTTCTTC CTCTATAGCA |
| 1151 | AGCTCACCGT GGACAAGAGC AGGTGGCAGC AGGGGAACGT CTTCTCATGC |
| 1201 | TCCGTGATGC ATGAGGCTCT GCACAACCAC TACACGCAGA AGAGCCTCTC |
| 1251 | CCTGTCTCCG GGTAAATGA |
| hTβRII (G4S)2-hFc: Amino Acid Sequence | |
| (SEQ ID NO: 15) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSTHT |
| 201 | CPPCPAPELL GGPSVFLFPP KPKDTLMISR TPEVTCVVVD VSHEDPEVKF |
| 251 | NWYVDGVEVH NAKTKPREEQ YNSTYRVVSV LTVLHQDWLN GKEYKCKVSN |
| 301 | KALPAPIEKT ISKAKGQPRE PQVYTLPPSR EEMTKNQVSL TCLVKGFYPS |
| 351 | DIAVEWESNG QPENNYKTTP PVLDSDGSFF LYSKLTVDKS RWQQGNVFSC |
| 401 | SVMHEALHNH YTQKSLSLSP GK |
| hTβRII extended hinge-hFc: Nucleic Acid Sequence | |
| (SEQ ID NO: 16) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 20h | CAACAACGGT GCAGTCAAGT TTCCACAACT GTGTAAATTT TGTGATGTGA |
| 25h | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA CCCAAATCTT GTGACAAAAC TCACACATGC |
| 601 | CCACCGTGCC CAGCACCTGA ACTCCTGGGG GGACCGTCAG TCTTCCTCTT |
| 651 | CCCCCCAAAA CCCAAGGACA CCCTCATGAT CTCCCGGACC CCTGAGGTCA |
| 701 | CATGCGTGGT GGTGGACGTG AGCCACGAAG ACCCTGAGGT CAAGTTCAAC |
| 751 | TGGTACGTGG ACGGCGTGGA GGTGCATAAT GCCAAGACAA AGCCGCGGGA |
| 801 | GGAGCAGTAC AACAGCACGT ACCGTGTGGT CAGCGTCCTC ACCGTCCTGC |
| 851 | ACCAGGACTG GCTGAATGGC AAGGAGTACA AGTGCAAGGT CTCCAACAAA |
| 901 | GCCCTCCCAG CCCCCATCGA GAAAACCATC TCCAAAGCCA AAGGGCAGCC |
| 951 | CCGAGAACCA CAGGTGTACA CCCTGCCCCC ATCCCGGGAG GAGATGACCA |
| 1001 | AGAACCAGGT CAGCCTGACC TGCCTGGTCA AAGGCTTCTA TCCCAGCGAC |
| 1051 | ATCGCCGTGG AGTGGGAGAG CAATGGGCAG CCGGAGAACA ACTACAAGAC |
| 1101 | CACGCCTCCC GTGCTGGACT CCGACGGCTC CTTCTTCCTC TATAGCAAGC |
| 1151 | TCACCGTGGA CAAGAGCAGG TGGCAGCAGG GGAACGTCTT CTCATGCTCC |
| 1201 | GTGATGCATG AGGCTCTGCA CAACCACTAC ACGCAGAAGA GCCTCTCCCT |
| 1251 | GTCCCCGGGT AAATGA |
| hTβRII extended hinge-hFc: Amino Acid Sequence | |
| (SEQ ID NO: 17) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| l01 | TSICEKPQEV CVAVWRKNDE NITLETVCHD PKLPYHDFIL EDAASPKCIM |
| 15h | KEKKKPGETF FMCSCSSDEC NDNIIFSEEY NTSNPDTGGG PKSCDKTHTC |
| 201 | PPCPAPELLG GPSVFLFPPK PKDTLMISRT PEVTCVVVDV SHEDPEVKFN |
| 251 | WYVDGVEVHN AKTKPREEQY NSTYRVVSVL TVLHQDWLNG KEYKCKVSNK |
| 301 | ALPAPIEKTI SKAKGQPREP QVYTLPPSRE EMTKNQVSLT CLVKGFYPSD |
| 351 | IAVEWESNGQ PENNYKTTPP VLDSDGSFFL YSKLTVDKSR WQQGNVFSCS |
| 401 | VMHEALHNHY TQKSLSLSPG K |
| hTβRII (G4S)5-hFc: Amino Acid Sequence | |
| (SEQ ID NO: 44) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSGGG GSTHTCPPCP APELLGGPSV FLFPPKPKDT LMISRTPEVT |
| 251 | CVVVDVSHED PEVKFNWYVD GVEVHNAKTK PREEQYNSTY RVVSVLTVLH |
| 301 | QDWLNGKEYK CKVSNKALPA PIEKTISKAK GQPREPQVYT LPPSREEMTK |
| 351 | NQVSLTCLVK GFYPSDIAVE WESNGQPENN YKTTPPVLDS DGSFFLYSKL |
| 401 | TVDKSRWQQG NVFSCSVMHE ALHNHYTQKS LSLSPGK |
| hTβRII (G4S)6-hFc: Amino Acid Sequence | |
| (SEQ ID NO: 45) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSGGG GSGGGGSTHT CPPCPAPELL GGPSVFLFPP KPKDTLMISR |
| 251 | TPEVTCVVVD VSHEDPEVKF NWYVDGVEVH NAKTKPREEQ YNSTYRVVSV |
| 301 | LTVLHQDWLN GKEYKCKVSN KALPAPIEKT ISKAKGQPRE PQVYTLPPSR |
| 351 | EEMTKNQVSL TCLVKGFYPS DIAVEWESNG QPENNYKTTP PVLDSDGSFF |
| 401 | LYSKLTVDKS RWQQGNVFSC SVMHEALHNH YTQKSLSLSP GK |
| hTβRII (G4S)5-hFc: Nucleotide Sequence | |
| (SEQ ID NO: 46) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGAGGA GGTTCTGGTG GTGGAGGTTC TGGAGGTGGA |
| 601 | GGAAGTGGTG GAGGTGGTTC TGGAGGTGGT GGAAGTACTC ACACATGCCC |
| 651 | ACCGTGCCCA GCACCTGAAC TCCTGGGGGG ACCGTCAGTC TTCCTCTTCC |
| 701 | CCCCAAAACC CAAGGACACC CTCATGATCT CCCGGACCCC TGAGGTCACA |
| 751 | TGCGTGGTGG TGGACGTGAG CCACGAAGAC CCTGAGGTCA AGTTCAACTG |
| 801 | GTACGTGGAC GGCGTGGAGG TGCATAATGC CAAGACAAAG CCGCGGGAGG |
| 851 | AGCAGTACAA CAGCACGTAC CGTGTGGTCA GCGTCCTCAC CGTCCTGCAC |
| 901 | CAGGACTGGC TGAATGGCAA GGAGTACAAG TGCAAGGTCT CCAACAAAGC |
| 951 | CCTCCCAGCC CCCATCGAGA AAACCATCTC CAAAGCCAAA GGGCAGCCCC |
| 1001 | GAGAACCACA GGTGTACACC CTGCCCCCAT CCCGGGAGGA GATGACCAAG |
| 1051 | AACCAGGTCA GCCTGACCTG CCTGGTCAAA GGCTTCTATC CCAGCGACAT |
| 1101 | CGCCGTGGAG TGGGAGAGCA ATGGGCAGCC GGAGAACAAC TACAAGACCA |
| 1151 | CGCCTCCCGT GCTGGACTCC GACGGCTCCT TCTTCCTCTA TAGCAAGCTC |
| 1201 | ACCGTGGACA AGAGCAGGTG GCAGCAGGGG AACGTCTTCT CATGCTCCGT |
| 1251 | GATGCATGAG GCTCTGCACA ACCACTACAC GCAGAAGAGC CTCTCCCTGT |
| 1301 | CTCCGGGTAA ATGA |
| hTβRII (G4S)6-hFc: Nucleotide Sequence | |
| (SEQ ID NO: 47) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGAGGT GGAAGTGGTG GAGGAGGTTC TGGTGGTGGA |
| 601 | GGTTCTGGAG GTGGAGGAAG TGGTGGAGGT GGTTCTGGAG GTGGTGGAAG |
| 651 | TACTCACACA TGCCCACCGT GCCCAGCACC TGAACTCCTG GGGGGACCGT |
| 701 | CAGTCTTCCT CTTCCCCCCA AAACCCAAGG ACACCCTCAT GATCTCCCGG |
| 751 | ACCCCTGAGG TCACATGCGT GGTGGTGGAC GTGAGCCACG AAGACCCTGA |
| 801 | GGTCAAGTTC AACTGGTACG TGGACGGCGT GGAGGTGCAT AATGCCAAGA |
| 851 | CAAAGCCGCG GGAGGAGCAG TACAACAGCA CGTACCGTGT GGTCAGCGTC |
| 901 | CTCACCGTCC TGCACCAGGA CTGGCTGAAT GGCAAGGAGT ACAAGTGCAA |
| 951 | GGTCTCCAAC AAAGCCCTCC CAGCCCCCAT CGAGAAAACC ATCTCCAAAG |
| 1001 | CCAAAGGGCA GCCCCGAGAA CCACAGGTGT ACACCCTGCC CCCATCCCGG |
| 1051 | GAGGAGATGA CCAAGAACCA GGTCAGCCTG ACCTGCCTGG TCAAAGGCTT |
| 1101 | CTATCCCAGC GACATCGCCG TGGAGTGGGA GAGCAATGGG CAGCCGGAGA |
| 1151 | ACAACTACAA GACCACGCCT CCCGTGCTGG ACTCCGACGG CTCCTTCTTC |
| 1201 | CTCTATAGCA AGCTCACCGT GGACAAGAGC AGGTGGCAGC AGGGGAACGT |
| 1251 | CTTCTCATGC TCCGTGATGC ATGAGGCTCT GCACAACCAC TACACGCAGA |
| 1301 | AGAGCCTCTC CCTGTCTCCG GGTAAATGA |

The various constructs were successfully expressed in CHO cells and were purified to a high degree of purity as determined by analytical size-exclusion chromatography and SDS-PAGE. The hTβRII (G4S)2-hFc, hTβRII (G4S)3-hFc, hTβRII (G4S)4-hFc, hTβRII (G4S)5-hFc and hTβRII (G4S)6-hFc proteins displayed similarly strong stability as determined by SDS-PAGE analysis when maintained in PBS for 13 days at 37° C. The hTβRII (G4S)2-hFc, hTβRII (G4S)3-hFc, hTβRII (G4S)4-hFc proteins were also maintained in rat, mouse or human serum and displayed similarly strong stability.

### TβRII ECD Variants

In addition to the TβRII domains included in the fusion proteins described above (e.g., SEQ ID NO: 18), the disclosure also contemplates fusion proteins comprising alternative TβRII domains. For example, the fusion protein may comprise the wild-type hTβRIIₛₕₒᵣₜ(23-159) sequence shown below (SEQ ID NO: 27) or any of the other TβRII polypeptides disclosed below:

| (SEQ ID NO: 27) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE YNTSNPD |

(1) The hTβRIIₛₕₒᵣₜ(23-159/D110K) amino acid sequence shown below (SEQ ID NO: 36), in which the substituted residue is underlined.

| (SEQ ID NO: 36) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHKFI LEDAASPKCI |
| 101 | MKEKKKPGET FFMCSCSSDE CNDNIIFSEE YNTSNPD |

(2) The N-terminally truncated hTβRIIₛₕₒᵣₜ(29-159) amino acid sequence shown below (SEQ ID NO: 28).

| (SEQ ID NO: 28) | |
|---|---|
| 1 | QKSVNNDMIV TDNNGAVKFP QLCKFCDVRF STCDNQKSCM SNCSITSICE |
| 51 | KPQEVCVAVW RKNDENITLE TVCHDPKLPY HDFILEDAAS PKCIMKEKKK |
| 101 | PGETFFMCSC SSDECNDNII FSEEYNTSNP D |

(3) The N-terminally truncated hTβRIIₛₕₒᵣₜ(35-159) amino acid sequence shown below (SEQ ID NO: 29).

| (SEQ ID NO: 29) | |
|---|---|
| 1 | DMIVTDNNGA VKFPQLCKFC DVRFSTCDNQ KSCMSNCSIT SICEKPQEVC |
| 51 | VAVWRKNDEN ITLETVCHDP KLPYHDFILE DAASPKCIMK EKKKPGETFF |
| 101 | MCSCSSDECN DNIIFSEEYN TSNPD |

(4) The C-terminally truncated hTβRIIₛₕₒᵣₜ(23-153) amino acid sequence shown below (SEQ ID NO: 30).

| (SEQ ID NO: 30) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE Y |

(5) The C-terminally truncated hTβRIIₛₕₒᵣₜ(23-153/N70D) amino acid sequence shown below (SEQ ID NO: 38), in which the substituted residue is underlined.

| (SEQ ID NO: 38) | |
|---|---|
| 1 | TIPPHVQKSV NNDMIVTDNN GAVKFPQLCK FCDVRFSTCD NQKSCMSDCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE Y |

Applicants also envision five corresponding variants (SEQ ID NOs: 37, 33, 34, 39) based on the wild-type hTβRII_{long}(23-184) sequence shown above and below (SEQ ID NO: 20), in which the 25 amino-acid insertion is underlined. Note that splicing results in a conservative amino acid substitution (Val→Ile) at the flanking position C-terminal to the insertion.

| (SEQ ID NO: 20) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PD |

(1) The hTβRII_{long}(23-184/D135K) amino acid sequence shown below (SEQ ID NO: 37), in which the substituted residue is double underlined.

| (SEQ ID NO: 37) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHKFILEDAA SPKCIMKEKK KPGETFFMCS CSSDECNDNI |
| 151 | IFSEEYNTSN PD |

(2) The N-terminally truncated hTβRII_{long}(29-184) amino acid sequence shown below (SEQ ID NO: 33).

| (SEQ ID NO: 33) | |
|---|---|
| 1 | QKSDVEMEAQ KDEIICPSCN RTAHPLRHIN NDMIVTDNNG AVKFPQLCKF |
| 51 | CDVRFSTCDN QKSCMSNCSI TSICEKPQEV CVAVWRKNDE NITLETVCHD |
| 101 | PKLPYHDFIL EDAASPKCIM KEKKKPGETF FMCSCSSDEC NDNIIFSEEY |
| 151 | NTSNPD |

(3) The N-terminally truncated hTβRII_{long}(60-184) amino acid sequence shown below (same as SEQ ID NO: 29).

| (same as SEQ ID NO: 29) | |
|---|---|
| 1 | DMIVTDNNGA VKFPQLCKFC DVRFSTCDNQ KSCMSNCSIT SICEKPQEVC |
| 51 | VAVWRKNDEN ITLETVCHDP KLPYHDFILE DAASPKCIMK EKKKPGETFF |
| 101 | MCSCSSDECN DNIIFSEEYN TSNPD |

(4) The C-terminally truncated hTβRII_{long}(23-178) amino acid sequence shown below (SEQ ID NO: 34).

| (SEQ ID NO: 34) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEY |

(5) The C-terminally truncated hTβRII_{long}(23-178/N95D) amino acid sequence shown below (SEQ ID NO: 39), in which the substituted residue is double underlined.

| (SEQ ID NO: 39) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSDCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEY |

Additional TβRII ECD variants include:

(A) The N- and C-terminally truncated hTβRIIₛₕₒᵣₜ(35-153) or hTβRII_{long}(60-178) amino acid sequence shown below (SEQ ID NO: 32).

| (SEQ ID NO: 32) | |
|---|---|
| 1 | DMIVTDNNGA VKFPQLCKFC DVRFSTCDNQ KSCMSNCSIT SICEKPQEVC |
| 51 | VAVWRKNDEN ITLETVCHDP KLPYHDFILE DAASPKCIMK EKKKPGETFF |
| 101 | MCSCSSDECN DNIIFSEEY |

(B) The N- and C-terminally truncated hTβRIIₛₕₒᵣₜ(29-153) amino acid sequence shown below (SEQ ID NO: 31).

| (SEQ ID NO: 31) | |
|---|---|
| 1 | QKSVNNDMIV TDNNGAVKFP QLCKFCDVRF STCDNQKSCM SNCSITSICE |
| 51 | KPQEVCVAVW RKNDENITLE TVCHDPKLPY HDFILEDAAS PKCIMKEKKK |
| 101 | PGETFFMCSC SSDECNDNII FSEEY |

(C) The N- and C-terminally truncated hTβRII_{long}(29-178) amino acid sequence shown below (SEQ ID NO: 35).

| (SEQ ID NO: 35) | |
|---|---|
| 1 | QKSDVEMEAQ KDEIICPSCN RTAHPLRHIN NDMIVTDNNG AVKFPQLCKF |
| 51 | CDVRFSTCDN QKSCMSNCSI TSICEKPQEV CVAVWRKNDE NITLETVCHD |
| 101 | PKLPYHDFIL EDAASPKCIM KEKKKPGETF FMCSCSSDEC NDNIIFSEEY |

Any of the above variants (SEQ ID NOs: 36, 28, 29, 30, 38, 37, 33, 34, 39, 32, 31, and 35) could incorporate an insertion of 36 amino acids (SEQ ID NO: 41) between the pair of glutamate residues (positions 151 and 152 of SEQ ID NO: 1, or positions 176 and 177 of SEQ ID NO: 2) located near the C-terminus of the hTβRII ECD, as occurs naturally in the hTβRII isoform C (Konrad et al., BMC Genomics 8:318, 2007).

(SEQ ID NO: 41)
GRCKIRHIGS NNRLQRSTCQ NTGWESAHVM KTPGFR

As an example, the paired glutamate residues flanking the optional insertion site are denoted below (underlined) for the hTβRIIₛₕₒᵣₜ(29-159) variant (SEQ ID NO: 28).

| (SEQ ID NO: 28) | |
|---|---|
| 1 | QKSVNNDMIV TDNNGAVKFP QLCKFCDVRF STCDNQKSCM SNCSITSICE |
| 51 | KPQEVCVAVW RKNDENITLE TVCHDPKLPY HDFILEDAAS PKCIMKEKKK |
| 101 | PGETFFMCSC SSDECNDNII FSEEYNTSNP D |

### Fc Domain Variants

While the constructs described above were generated with an Fc domain having the amino acid sequence of SEQ ID NO: 49, the disclosure contemplates hTβRII-hFc fusion proteins comprising alternative Fc domains, including a human IgG₂ Fc domain (SEQ ID NO: 42, below) or full-length human IgG₁ Fc (hG1Fc) (SEQ ID NO: 43, below). Optionally, a polypeptide unrelated to an Fc domain could be attached in place of the Fc domain.

| (SEQ ID NO: 42) | |
|---|---|
| 1 | VECPPCPAPP VAGPSVFLFP PKPKDTLMIS RTPEVTCVVV DVSHEDPEVQ |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |
| (SEQ ID NO: 43) | |
| 1 | GGPKSCDKTH TCPPCPAPEL LGGPSVFLFP PKPKDTLMIS RTPEVTCVVV |
| 51 | DVSHEDPEVK FNWYVDGVEV HNAKTKPREE QYNSTYRVVS VLTVLHQDWL |
| 101 | NGKEYKCKVS NKALPAPIEK TISKAKGQPR EPQVYTLPPS REEMTKNQVS |
| 151 | LTCLVKGFYP SDIAVEWESN GQPENNYKTT PPVLDSDGSF FLYSKLTVDK |
| 201 | SRWQQGNVFS CSVMHEALHN HYTQKSLSLS PGK |

### Leader Sequence Variants

While the generated constructs described above included the TPA leader sequence, alternative leader sequences may be used, such as the native leader sequence (SEQ ID NO: 22-below) or the honey bee melittin (SEQ ID NO: 24-below) leader sequences.

Native:
(SEQ ID NO: 22)
MGRGLLRGLTNPLHIVLYNTRIAS
Honey bee melittin (HBML):
(SEQ ID NO: 24
MKFLVNVALVFMVVYISYIYA

### Example 2. Differential Ligand Inhibition by Receptor Fusion Protein Variants in Cell-Based Assay

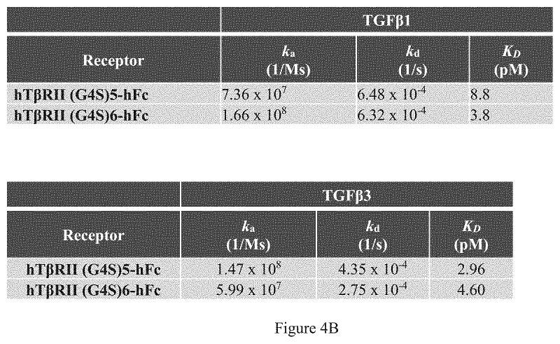

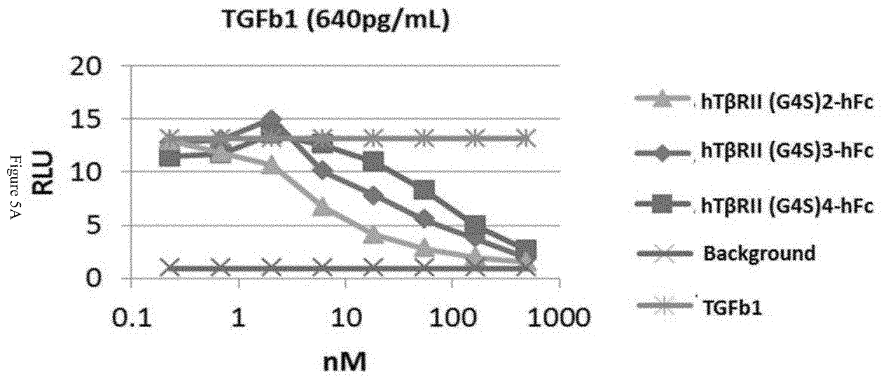

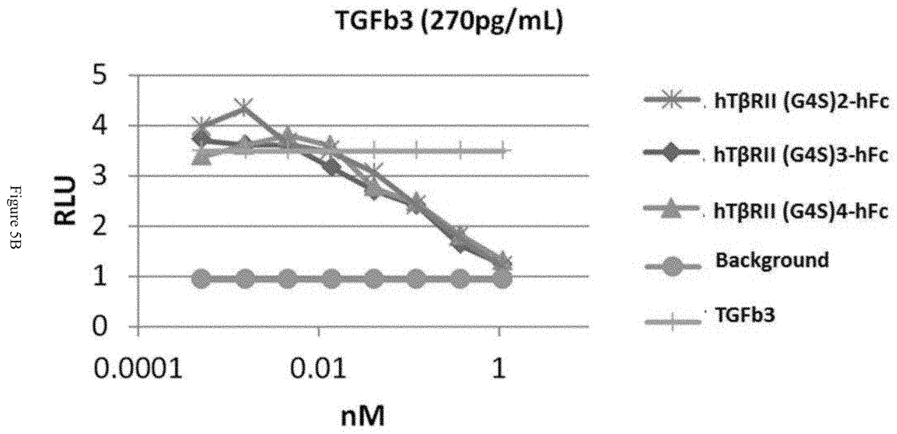

Affinities of TGFβ1, TGFβ2 and TGFβ3 for hTβRII (G4S)2-hFc; hTβRII (G4S)3-hFc; hTβRII (G4S)4-hFc; hTβRII-hFc; and hTβRII extended hinge-hFc proteins were evaluated in vitro with a Biacore™ instrument, and the results are summarized in FIGS. 4A and 4B. Each of the fusion proteins was capable of binding TGFβ1 and TGFβ3 with high affinity, but the constructs having linker lengths longer than or equal to (G4S)4 were surprisingly capable of binding to both TGFβ1 and TGFβ3 with higher affinity than constructs having linker lengths shorter than (G4S)4. Binding between TGFβ2 and any of the constructs was low or transient. Deglycosylation of the constructs did not change binding.

A reporter gene assay in A549 cells was used to determine the ability of hTβRII-hFc variants to inhibit activity of TGFβ1, TGFβ2 and TGFβ3. This assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates. On the second day, a solution containing pGL3(CAGA)12, pRLCMV, X-tremeGENE 9 (Roche Applied Science), and OptiMEM (Invitrogen) was preincubated, then added to Eagle's minimum essential medium (EMEM, ATCC®) supplemented with 0.1% BSA, which was applied to the plated cells for incubation overnight at 37° C., 5% CO₂. On the third day, medium was removed, and cells were incubated overnight at 37° C., 5% CO₂ with a mixture of ligands and inhibitors prepared as described below.

Serial dilutions of test articles were made in a 48-well plate in assay buffer (EMEM+0.1% BSA). An equal volume of assay buffer containing the test ligand was added to obtain a final ligand concentration equal to the EC50 determined previously. Human TGFβ1, human TGFβ2, and human TGFβ3 were obtained from PeproTech. Test solutions were incubated at 37° C. for 30 minutes, then a portion of the mixture was added to all wells. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred in duplicate to a chemiluminescence plate (96-well) and analyzed in a luminometer with reagents from a Dual-Luciferase Reporter Assay system (Promega E1980) to determine normalized luciferase activity.

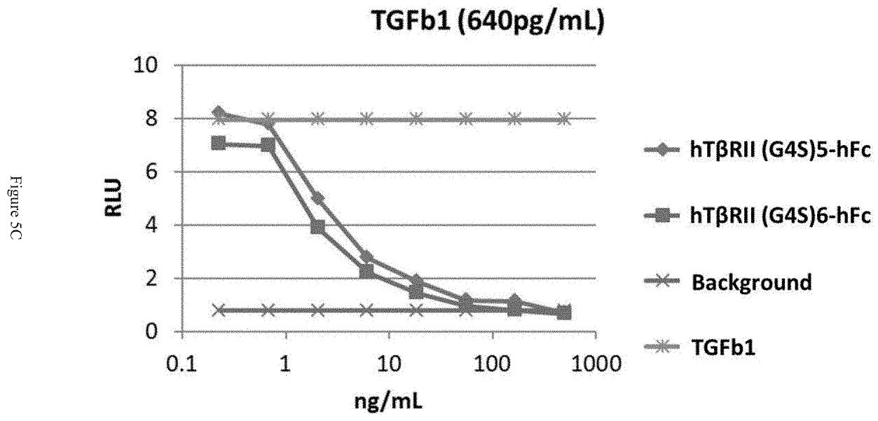

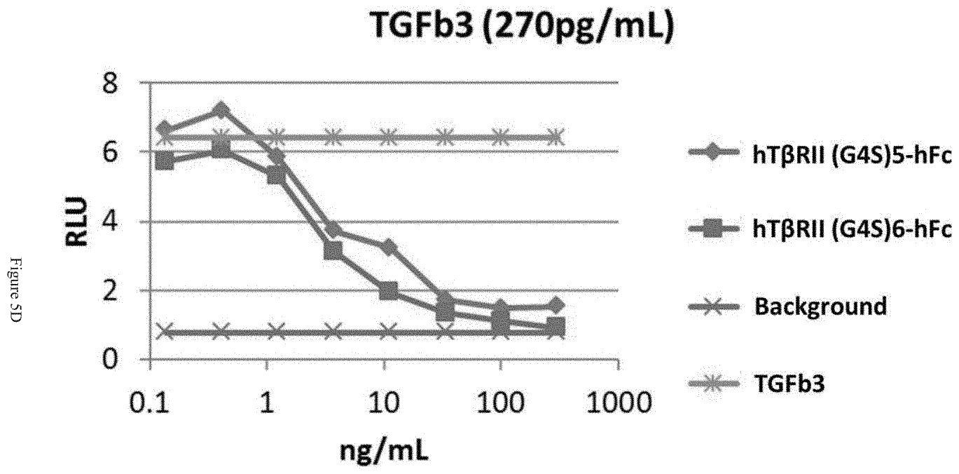

As illustrated in FIGS. 5A-5F, the hTβRII (G4S)2-hFc; hTβRII (G4S)3-hFc; hTβRII (G4S)4-hFc; hTβRII (G4S)5-hFc; hTβRII (G4S)6-hFc; hTβRII-hFc; and hTβRII extended hinge-hFc proteins all were capable of inhibiting both TGFβ1 and TGFβ3. Interestingly, while there was a correlation between improved TGFβ1 and TGFβ3 inhibition and linker length for the the hTβRII (G4S)2-hFc; hTβRII (G4S)3-hFc and hTβRII (G4S)4-hFc constructs (FIG. 5E), this improvement trend appeared to have plateaued for hTβRII (G4S)5-hFc and hTβRII (G4S)6-hFc constructs (FIG. 5F).

### Example 3. Generation of an ActRIIB:TβRII Heterodimer

Soluble ActRIIB-Fc:TβRII-Fc heteromeric complexes comprising the extracellular domains of human ActRIIB and human TβRII, which are each separately fused to an Fc domain with a linker positioned between the extracellular domain and the Fc domain, were constructed. The individual constructs are referred to as ActRIIB-Fc fusion polypeptide and TβRII-Fc fusion polypeptide, respectively, and the sequences for each are provided below.

A methodology for promoting formation of ActRIIB-Fc:TβRII-Fc heteromeric complexes, as opposed to ActRIIB-Fc or TβRII-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB-Fc and TβRII-Fc polypeptide sequences of SEQ ID NOs: 82, 84, 85, and 87, respectively, one Fc domain is altered to introduce cationic amino acids at the interaction face, while the other Fc domain is altered to introduce anionic amino acids at the interaction face. ActRIIB-Fc fusion polypeptide and TβRII-Fc fusion polypeptide each employ the tissue plasminogen activator (TPA) leader (SEQ ID NO: 23) and a (G4S)4 linker positioned between the ActRIIB or TβRII extracellular portion and the modified Fc portion.

The ActRIIB-Fc polypeptide sequence (SEQ ID NO: 82) is shown below:

| (SEQ ID NO: 82) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGGSGGGGS |
| 151 | GGGGSGGGGS THTCPPCPAP ELLGGPSVFL FPPKPKDTLM ISRTPEVTCV |
| 201 | VVDVSHEDPE VKFNWYVDGV EVHNAKTKPR EEQYNSTYRV VSVLTVLHQD |
| 251 | WLNGKEYKCK VSNKALPAPI EKTISKAKGQ PREPQVYTLP PSRKEMTKNQ |
| 301 | VSLTCLVKGF YPSDIAVEWE SNGQPENNYK TTPPVLKSDG SFFLYSKLTV |
| 351 | DKSRWQQGNV FSCSVMHEAL HNHYTQKSLS LSPGK |

The leader (signal) sequence and linker are underlined. To promote formation of ActRIIB-Fc:TβRII-Fc heterodimer rather than either of the possible homodimeric complexes, two amino acid substitutions (replacing acidic amino acids with lysine) can be introduced into the Fc domain of the ActRIIB fusion protein as indicated by double underline above. The amino acid sequence of SEQ ID NO: 82 may optionally be provided with lysine (K) removed from the C-terminus.

This ActRIIB-Fc fusion protein is encoded by the following nucleic acid sequence (SEQ ID NO: 83):

| (SEQ ID NO: 83) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAG GTTCTGGAGG TGGAGGAAGT |
| 451 | GGTGGAGGTG GTTCTGGAGG TGGTGGAAGT ACTCACACAT GCCCACCGTG |
| 501 | CCCAGCACCT GAACTCCTGG GGGGACCGTC AGTCTTCCTC TTCCCCCCAA |
| 551 | AACCCAAGGA CACCCTCATG ATCTCCCGGA CCCCTGAGGT CACATGCGTG |
| 601 | GTGGTGGACG TGAGCCACGA AGACCCTGAG GTCAAGTTCA ACTGGTACGT |
| 651 | GGACGGCGTG GAGGTGCATA ATGCCAAGAC AAAGCCGCGG GAGGAGCAGT |
| 701 | ACAACAGCAC GTACCGTGTG GTCAGCGTCC TCACCGTCCT GCACCAGGAC |
| 751 | TGGCTGAATG GCAAGGAGTA CAAGTGCAAG GTCTCCAACA AAGCCCTCCC |
| 801 | AGCCCCCATC GAGAAAACCA TCTCCAAAGC CAAAGGGCAG CCCCGAGAAC |
| 851 | CACAGGTGTA CACCCTGCCC CCATCCCGGA AGGAGATGAC CAAGAACCAG |
| 901 | GTCAGCCTGA CCTGCCTGGT CAAAGGCTTC TATCCCAGCG ACATCGCCGT |
| 951 | GGAGTGGGAG AGCAATGGGC AGCCGGAGAA CAACTACAAG ACCACGCCTC |
| 1001 | CCGTGCTGAA GTCCGACGGC TCCTTCTTCC TCTATAGCAA GCTCACCGTG |
| 1051 | GACAAGAGCA GGTGGCAGCA GGGGAACGTC TTCTCATGCT CCGTGATGCA |
| 1101 | TGAGGCTCTG CACAACCACT ACACGCAGAA GAGCCTCTCC CTGTCTCCGG |
| 1151 | GTAAATGA |

The processed ActRIIB-Fc fusion polypeptide (SEQ ID NO: 84) is as follows, and may optionally be provided with lysine (K) removed from the C-terminus.

| (SEQ ID NO: 84) | |
|---|---|
| 1 | GRGEAETREC IYYNANWELE RTNQSGLERC EGEQDKRLHC YASWRNSSGT IELVKKGCWL |
| 61 | DDFNCYDRQE CVATEENPQV YFCCCEGNFC NERFTHLPEA GGPEVTYEPP PTAPTGGGGS |
| 121 | GGGGSGGGGS GGGGSTHTCP PCPAPELLGG PSVFLFPPKP KDTLMISRTP EVTCVVVDVS |
| 181 | HEDPEVKFNW YVDGVEVHNA KTKPREEQYN STYRVVSVLT VLHQDWLNGK EYKCKVSNKA |
| 241 | LPAPIEKTIS KAKGQPREPQ VYTLPPSRKE MTKNQVSLTC LVKGFYPSDI AVEWESNGQP |
| 301 | ENNYKTTPPV LKSDGSFFLY SKLTVDKSRW QQGNVFSCSV MHEALHNHYT QKSLSLSPGK |

The complementary form of TβRII-Fc fusion polypeptide (SEQ ID NO: 85) is as follows:

| (SEQ ID NO: 85) | |
|---|---|
| 1 | MDAMKRGLCC VLLLCGAVFV SPGATIPPHV QKSDVEMEAQ KDEITCPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSTHT CPPCPAPELL GGPSVFLFPP KPKDTLMISR TPEVTCVVVD |
| 251 | VSHEDPEVKF NWYVDGVEVH NAKTKPREEQ YNSTYRVVSV LTVLHQDWLN |
| 301 | GKEYKCKVSN KALPAPIEKT ISKAKGQPRE PQVYTLPPSR EEMTKNQVSL |
| 351 | TCLVKGFYPS DIAVEWESNG QPENNY [symbol] TTP PVLDSDGSFF LYS [symbol] LTVDKS |
| 401 | RWQQGNVFSC SVMHEALHNH YTQKSLSLSP GK |

The leader sequence and linker are underlined. To guide heterodimer formation with the ActRIIB-Fc fusion polypeptide of SEQ ID NOs: 82 and 84 above, two amino acid substitutions (replacing lysines with aspartic acids) can be introduced into the Fc domain of the TβRII-Fc fusion polypeptide as indicated by double underline above. The amino acid sequence of SEQ ID NO: 85 may optionally be provided with lysine (K) added at the C-terminus.

This TβRII-Fc fusion protein is encoded by the following nucleic acid (SEQ ID NO: 86):

| (SEQ ID NO: 86) | |
|---|---|
| 1 | ATGGATGCGA TGAAACGCGG CCTGTGCTGC GTGCTGCTGC TGTGCGGCGC |
| 51 | GGTGTTTGTG AGCCCGGGCG CCACCATTCC GCCGCATGTG CAGAAAAGCG |
| 101 | ATGTGGAAAT GGAAGCGCAG AAAGATGAAA TTATTTGCCC GAGCTGCAAC |
| 151 | CGCACCGCGC ATCCGCTGCG CCATATTAAC AACGATATGA TTGTGACCGA |
| 201 | TAACAACGGC GCGGTGAAAT TTCCGCAGCT GTGCAAATTT TGCGATGTGC |
| 251 | GCTTTAGCAC CTGCGATAAC CAGAAAAGCT GCATGAGCAA CTGCAGCATT |
| 301 | ACCAGCATTT GCGAAAAACC GCAGGAAGTG TGCGTGGCGG TGTGGCGCAA |
| 351 | AAACGATGAA AACATTACCC TGGAAACCGT GTGCCATGAT CCGAAACTGC |
| 401 | CGTATCATGA TTTTATTCTG GAAGATGCGG CGAGCCCGAA ATGCATTATG |
| 451 | AAAGAAAAAA AAAAACCGGG CGAAACCTTT TTTATGTGCA GCTGCAGCAG |
| 501 | CGATGAATGC AACGATAACA TTATTTTTAG CGAAGAATAT AACACCAGCA |
| 551 | ACCCGGATAC CGGTGGCGGC GGCAGCGGCG GCGGCGGCAG CGGCGGCGGC |
| 601 | GGCAGCGGCG GCGGCGGCAG CACCCATACC TGCCCGCCGT GCCCGGCGCC |
| 651 | GGAACTGCTG GGCGGCCCGA GCGTGTTTCT GTTTCCGCCG AAACCGAAAG |
| 701 | ATACCCTGAT GATTAGCCGC ACCCCGGAAG TGACCTGCGT GGTGGTGGAT |
| 751 | GTGAGCCATG AAGATCCGGA AGTGAAATTT AACTGGTATG TGGATGGCGT |
| 801 | GGAAGTGCAT AACGCGAAAA CCAAACCGCG CGAAGAACAG TATAACAGCA |
| 851 | CCTATCGCGT GGTGAGCGTG CTGACCGTGC TGCATCAGGA TTGGCTGAAC |
| 901 | GGCAAAGAAT ATAAATGCAA AGTGAGCAAC AAAGCGCTGC CGGCGCCGAT |
| 951 | TGAAAAAACC ATTAGCAAAG CGAAAGGCCA GCCGCGCGAA CCGCAGGTGT |
| 1001 | ATACCCTGCC GCCGAGCCGC GAAGAAATGA CCAAAAACCA GGTGAGCCTG |
| 1051 | ACCTGCCTGG TGAAAGGCTT TTATCCGAGC GATATTGCGG TGGAATGGGA |
| 1101 | AAGCAACGGC CAGCCGGAAA ACAACTATGA TACCACCCCG CCGGTGCTGG |
| 1151 | ATAGCGATGG CAGCTTTTTT CTGTATAGCG ATCTGACCGT GGATAAAAGC |
| 1201 | CGCTGGCAGC AGGGCAACGT GTTTAGCTGC AGCGTGATGC ATGAAGCGCT |
| 1251 | GCATAACCAT TATACCCAGA AAAGCCTGAG CCTGAGCCCG GGCGATGATG |
| 1301 | ATGATAAAGC GCATCATCAT CATCATCATT AA |

The processed TβRII-Fc fusion protein sequence (SEQ ID NO: 87) is as follows and may optionally be provided with lysine (K) added at the C-terminus.

| (SEQ ID NO: 87) | |
|---|---|
| 1 | TIPPHVQKSD VEMEAQKDEI ICPSCNRTAH PLRHINNDMI VTDNNGAVKF PQLCKFCDVR |
| 61 | FSTCDNQKSC MSNCSITSIC EKPQEVCVAV WRKNDENITL ETVCHDPKLP YHDFILEDAA |
| 121 | SPKCIMKEKK KPGETFFMCS CSSDECNDNI IFSEEYNTSN PDTGGGGSGG GGSGGGGSGG |
| 181 | GGSTHTCPPC PAPELLGGPS VFLFPPKPKD TLMISRTPEV TCVVVDVSHE DPEVKFNWYV |
| 241 | DGVEVHNAKT KPREEQYNST YRVVSVLTVL HQDWLNGKEY KCKVSNKALP APIEKTISKA |
| 301 | KGQPREPQVY TLPPSREEMT KNQVSLTCLV KGFYPSDIAV EWESNGQPEN NYDTTPPVLD |
| 361 | SDGSFFLYSD LTVDKSRWQQ GNVFSCSVMH EALHNHYTQK SLSLSPGK |

The ActRIIB-Fc and TβRII-Fc proteins of SEQ ID NO: 84 and SEQ ID NO: 87, respectively, may be co-expressed and purified from a CHO cell line, to give rise to a heteromeric complex comprising ActRIIB-Fc:TβRII-Fc.

In another approach to promote the formation of heteromultimer complexes using asymmetric Fc fusion proteins the Fc domains are altered to introduce complementary hydrophobic interactions and an additional intermolecular disulfide bond as illustrated in the ActRIIB-Fc and TβRII-Fc polypeptide sequences of SEQ ID NOs: 88-90 and 91-93, respectively. The ActRIIB-Fc fusion polypeptide and TβRII-Fc fusion polypeptide each employ the tissue plasminogen activator (TPA) leader.

The ActRIIB-Fc polypeptide sequence (SEQ ID NO: 88) is shown below:

| (SEQ ID NO: 88) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGGSGGGGS |
| 151 | GGGGSGGGGS THTCPPCPAP ELLGGPSVFL FPPKPKDTLM ISRTPEVTCV |
| 201 | VVDVSHEDPE VKFNWYVDGV EVHNAKTKPR EEQYNSTYRV VSVLTVLHQD |
| 251 | WLNGKEYKCK VSNKALPAPI EKTISKAKGQ PREPQVYTLP PCREEMTKNQ |
| 301 | VSLWCLVKGF YPSDIAVEWE SNGQPENNYK TTPPVLDSDG SFFLYSKLTV |
| 351 | DKSRWQQGNV FSCSVMHEAL HNHYTQKSLS LSPGK |

The leader (signal) sequence and linker are underlined. To promote formation of the ActRIIB-Fc:TβRII-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 88 may optionally be provided with lysine (K) removed from the C-terminus.

This ActRIIB-Fc fusion protein is encoded by the following nucleic acid sequence (SEQ ID NO: 89):

| (SEQ ID NO: 89) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAG GTTCTGGAGG TGGAGGAAGT |
| 451 | GGTGGAGGTG GTTCTGGAGG TGGTGGAAGT ACTCACACAT GCCCACCGTG |
| 501 | CCCAGCACCT GAACTCCTGG GGGGGCCGTC AGTCTTCCTC TTCCCCCCAA |
| 551 | AACCCAAGGA CACCCTCATG ATCTCCCGGA CCCCTGAGGT CACATGCGTG |
| 601 | GTGGTGGACG TGAGCCACGA AGACCCTGAG GTCAAGTTCA ACTGGTACGT |
| 651 | GGACGGCGTG GAGGTGCATA ATGCCAAGAC AAAGCCGCGG GAGGAGCAGT |
| 701 | ACAACAGCAC GTACCGTGTG GTCAGCGTCC TCACCGTCCT GCACCAGGAC |
| 751 | TGGCTGAATG GCAAGGAGTA CAAGTGCAAG GTCTCCAACA AAGCCCTCCC |
| 801 | AGCCCCCATC GAGAAAACCA TCTCCAAAGC CAAAGGGCAG CCCCGAGAAC |
| 851 | CACAGGTGTA CACCCTGCCC CCATGCCGGG AGGAGATGAC CAAGAACCAG |
| 901 | GTCAGCCTGT GGTGCCTGGT CAAAGGCTTC TATCCCAGCG ACATCGCCGT |
| 951 | GGAGTGGGAG AGCAATGGGC AGCCGGAGAA CAACTACAAG ACCACGCCTC |
| 1001 | CCGTGCTGGA CTCCGACGGC TCCTTCTTCC TCTATAGCAA GCTCACCGTG |
| 1051 | GACAAGAGCA GGTGGCAGCA GGGGAACGTC TTCTCATGCT CCGTGATGCA |
| 1101 | TGAGGCTCTG CACAACCACT ACACGCAGAA GAGCCTCTCC CTGTCTCCGG |
| 1151 | GTAAATGA |

The processed ActRIIB-Fc fusion polypeptide is as follows:

| (SEQ ID NO: 90) | |
|---|---|
| 1 | GRGEAETREC IYYNANWELE RTNQSGLERC EGEQDKRLHC YASWRNSSGT IELVKKGCWL |
| 61 | DDFNCYDRQE CVATEENPQV YFCCCEGNFC NERFTHLPEA GGPEVTYEPP PTAPTGGGGS |
| 121 | GGGGSGGGGS GGGGSTHTCP PCPAPFLLGG PSVFLFPPKP KDTLMISRTP EVTCVVVDVS |
| 181 | HEDPEVKFNW YVDGVEVHNA KTKPREEQYN STYRVVSVLT VLHQDWLNGK EYKCKVSNKA |
| 241 | LPAPIEKTIS KAKGQPREPQ VYTLPPCREE MTKNQVSLWC LVKGFYPSDI AVEWESNGQP |
| 301 | ENNYKTTPPV LDSDGSFFLY SKLTVDKSRW QQGNVFSCSV MHEALHNHYT QKSLSLSPGK |

The complementary form of TβRII-Fc fusion polypeptide (SEQ ID NO: 91) is as follows and may optionally be provided with lysine (K) removed from the C-terminus.

| (SEQ ID NO: 91) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSTHT CPPCPAPELL GGPSVFLFPP KPKDTLMISR TPEVTCVVVD |
| 251 | VSHEDPEVKF NWYVDGVEVH NAKTKPREEQ YNSTYRVVSV LTVLHQDWLN |
| 301 | GKEYKCKVSN KALPAPIEKT ISKAKGQPRE PQVCTLPPSR EEMTKNQVSL |
| 351 | SCAVKGFYPS DIAVEWESNG QPENNYKTTP PVLDSDGSFF LVSKLTVDKS |
| 401 | RWQQGNVFSC SVMHEALHNH YTQKSLSLSP GK |

The leader sequence and the linker are underlined. To guide heterodimer formation with the ActRIIB-Fc fusion polypeptide of SEQ ID NOs: 88 and 91 above, four amino acid substitutions can be introduced into the Fc domain of the TβRII fusion polypeptide as indicated by double underline above. The amino acid sequence of SEQ ID NO: 91 may optionally be provided with lysine (K) removed from the C-terminus.

This A TβRII-Fc fusion protein is encoded by the following nucleic acid sequence (SEQ ID NO: 92):

| (SEQ ID NO: 92) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA GGTTCTGGAG GTGGAGGAAG TGGTGGAGGT |
| 601 | GGTTCTGGAG GTGGTGGAAG TACTCACACA TGCCCACCGT GCCCAGCACC |
| 651 | TGAACTCCTG GGGGGACCGT CAGTCTTCCT CTTCCCCCCA AAACCCAAGG |
| 701 | ACACCCTCAT GATCTCCCGG ACCCCTGAGG TCACATGCGT GGTGGTGGAC |
| 751 | GTGAGCCACG AAGACCCTGA GGTCAAGTTC AACTGGTACG TGGACGGCGT |
| 801 | GGAGGTGCAT AATGCCAAGA CAAAGCCGCG GGAGGAGCAG TACAACAGCA |
| 851 | CGTACCGTGT GGTCAGCGTC CTCACCGTCC TGCACCAGGA CTGGCTGAAT |
| 901 | GGCAAGGAGT ACAAGTGCAA GGTCTCCAAC AAAGCCCTCC CAGCCCCCAT |
| 951 | CGAGAAAACC ATCTCCAAAG CCAAAGGGCA GCCCCGAGAA CCACAGGTGT |
| 1001 | GCACCCTGCC CCCATCCCGG GAGGAGATGA CCAAGAACCA GGTCAGCCTG |
| 1051 | TCCTGCGCCG TCAAAGGCTT CTATCCCAGC GACATCGCCG TGGAGTGGGA |
| 1101 | GAGCAATGGG CAGCCGGAGA ACAACTACAA GACCACGCCT CCCGTGCTGG |
| 1151 | ACTCCGACGG CTCCTTCTTC CTCGTGAGCA AGCTCACCGT GGACAAGAGC |
| 1201 | AGGTGGCAGC AGGGGAACGT CTTCTCATGC TCCGTGATGC ATGAGGCTCT |
| 1251 | GCACAACCAC TACACGCAGA AGAGCCTCTC CCTGTCTCCG GGTAAATGA |

A processed TβRII-Fc fusion protein sequence is as follows and may optionally be provided with lysine (K) removed from the C-terminus.

| (SEQ ID NO: 93) | |
|---|---|
| 1 | TIPPHVQKSD VEMEAQKDEI ICPSCNRTAH PLRHINNDMI VTDNNGAVKF PQLCKFCDVR |
| 61 | FSTCDNQKSC MSNCSITSIC EKPQEVCVAV WRKNDENITL ETVCHDPKLP YHDFILEDAA |
| 121 | SPKCIMKEKK KPGETFFMCS CSSDECNDNI IFSEEYNTSN PDTGGGGSGG GGSGGGGSGG |
| 161 | GGSTHTCPPC PAPELLGGPS VFLFPPKPKD TLMISRTPEV TCVVVDVSHE DPEVKFNWYV |
| 241 | DGVEVHNAKT KPREEQYNST YRVVSVLTVL HQDWLNGKEY KCKVSNKALP APIEKTISKA |
| 301 | KGQPREPQVC TLPPSREEMT KNQVSLSCAV KGFYPSDIAV EWESNGQPEN NYKTTPPVLD |
| 361 | SDGSFFLVSK LTVDKSRWQQ GNVFSCSVMH EALHNHYTQK SLSLSPGK |

ActRIIB-Fc and TβRII-Fc proteins of SEQ ID NO: 90 and SEQ ID NO: 93, respectively, may be co-expressed and purified from a CHO cell line, to give rise to a heteromeric complex comprising ActRIIB-Fc:TβRII-Fc.

In order to compare the activity of the ActRIIB-Fc:TβRII-Fc heterodimers, ActRIIB-Fc and TβRII-Fc homodimers were generated, which each comprise either the ActRIIB or TβRII extracellular domains as present in any one of SEQ ID NO: 82, 84, 85, 87, 88, 90, 91, or 93; an unmodified hG1Fc domain (promotes homodimer formation); and a (G4S)4 linker positioned between the ActRIIB or TβRII extracellular portion and the unmodified Fc portion. Both of these homodimers were expressed using the TPA leader sequence of SEQ ID NO: 23.

Purification of various heterodimer and homodimers described above could be achieved by a series of column chromatography steps, including, for example, three or more of the following, in any order: protein A chromatography, Q sepharose chromatography, phenylsepharose chromatography, size exclusion chromatography and epitope-based affinity chromatography (e.g., with an antibody or functionally equivalent ligand directed against an epitope on TβRII or ActRIIB), and multimodal chromatography (e.g., with resin containing both electrostatic and hydrophobic ligands). The purification could be completed with viral filtration and buffer exchange.

### Example 4. Differential Ligand Inhibition by Receptor Fusion Protein Variants in Cell-Based Assay

A reporter gene assay in A549 cells was used to determine the ability of an ActRIIB-Fc:TβRII-Fc heterodimer to inhibit activity of TGFβ1, TGFβ2, TGFβ3, activin A, activin B, GDF11, GDF8, BMP9, and BMP10 and compared to the inhibiting activity of an ActRIIB-Fc homodimer and TβRII-Fc homodimer, which are all described above in Example 3. This assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates. On the second day, a solution containing pGL3(CAGA)12, pRLCMV, X-tremeGENE 9 (Roche Applied Science), and OptiMEM (Invitrogen) was preincubated, then added to Eagle's minimum essential medium (EMEM, ATCC®) supplemented with 0.1% BSA, which was applied to the plated cells for incubation overnight at 37° C., 5% CO₂. On the third day, medium was removed, and cells were incubated overnight at 37° C., 5% CO₂ with a mixture of ligands and inhibitors prepared as described below.

Serial dilutions of test articles were made in a 48-well plate in assay buffer (EMEM+0.1% BSA). An equal volume of assay buffer containing the test ligand was added to obtain a final ligand concentration equal to the EC50 determined previously. Test solutions were incubated at 37° C. for 30 minutes, then a portion of the mixture was added to all wells. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred in duplicate to a chemiluminescence plate (96-well) and analyzed in a luminometer with reagents from a Dual-Luciferase Reporter Assay system (Promega E1980) to determine normalized luciferase activity.

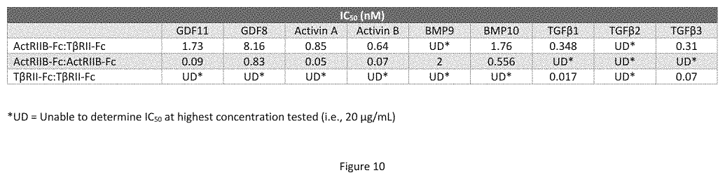

As illustrated in FIG. 10, the TβRII-Fc homodimer was capable of inhibiting TGFβ1 and TGFβ 3 in this cell-based assay but did not inhibit TGFβ2, activin A, activin B, GDF11, GDF8, BMP9, or BMP10. In contrast, the ActRIIB-Fc homodimer was capable of inhibiting activin A, activin B, GDF11, GDF8, BMP9, and BMP10 but did not inhibit TGFβ1, TGFβ2, or TGFβ3. The ActRIIB-Fc:TβRII-Fc heterodimer was capable of inhibiting TGFβ1, TGFβ3, activin A, activin B, GDF11, GDF8, and BMP10 but did not inhibit BMP9 or TGFβ2. These data demonstrate that ActRIIB-Fc:TβRII-Fc heterodimers retain many of potent inhibitor characteristics of ActRIIB-Fc and TβRII-Fc homodimers and thus represent an interesting class of ligand traps that are uniquely capable of affecting two distinct groups of Smad 2/3-related TGFβ superfamily ligands (i.e., the TGFβs and activin/GDFs). Moreover, the ActRIIB-Fc:TβRII-Fc heterodimer did not inhibit BMP9, and thus with respect to ActRIIB-associated ligands, ActRIIB-Fc:TβRII-Fc is a more selective antagonist than an ActRIIB homodimer. Accordingly, ActRIIB-Fc:TβRII-Fc heterodimers will be more useful than ActRIIB homodimer in certain applications where such selective antagonism is desired in combination with inhibition of TGFβ1 and TGFβ3.

### Example 5. Generation of an ActRIIA:TβRII Heterodimer

Soluble ActRIIA-Fc:TβRII-Fc heteromeric complexes comprising the extracellular domains of human ActRIIA and human TβRII, which are each separately fused to an Fc domain with a linker positioned between the extracellular domain and the Fc domain, were constructed. The individual constructs are referred to as ActRIIA-Fc fusion polypeptide and TβRII-Fc fusion polypeptide, respectively, and the sequences for each are provided below.

A methodology for promoting formation of ActRIIA-Fc:TβRII-Fc heteromeric complexes, as opposed to ActRIIA-Fc or TβRII-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIA-Fc and TβRII-Fc polypeptide sequences of SEQ ID NOs: 128, 130, 131, and 133, respectively, one Fc domain is altered to introduce cationic amino acids at the interaction face, while the other Fc domain is altered to introduce anionic amino acids at the interaction face. ActRIIA-Fc fusion polypeptide and TβRII-Fc fusion polypeptide each employ the tissue plasminogen activator (TPA) leader (SEQ ID NO: 23) and a (G4S)4 linker positioned between the ActRIIA or TβRII extracellular portion and the modified Fc portion.

The ActRIIA-Fc polypeptide sequence (SEQ ID NO: 128) is shown below:

| (SEQ ID NO: 128) | |
|---|---|
| 1 | MDAMKRGLCC VLLLCGAVFV SPGAAILGRS ETQECLFFNA NWEKDRTNQT |
| 51 | GVEPCYGDKD KRRHCFATWK NISGSIEIVK QGCWLDDINC YDRTDCVEKK |
| 101 | DSPEVYFCCC EGNMCNEKFS YFPEMEVTQP TSNPVTPKPP TGGGGSGGGG |
| 151 | SGGGGSGGGG STHTCPPCPA PELLGGPSVF LFPPKPKDTL MISRTPEVTC |
| 201 | VVVDVSHEDP EVKFNWYVDG VEVHNAKTKP REEQYNSTYR VVSVLTVLHQ |
| 251 | DWLNGKEYKC KVSNKALPAP IEKTISKAKG QPREPQVYTL PPSRKEMTKN |
| 301 | QVSLTCLVKG FYPSDIAVEW ESNGQPENNY KTTPPVLKSD GSFFLYSKLT |
| 351 | VDKSRWQQGN VFSCSVMHEA LHNHYTQKSL SLSPGK |

The leader (signal) sequence and linker are underlined. To promote formation of ActRIIA-Fc:TβRII-Fc heterodimer rather than either of the possible homodimeric complexes, two amino acid substitutions (replacing acidic amino acids with lysine) can be introduced into the Fc domain of the ActRIIA fusion protein as indicated by double underline above. The amino acid sequence of SEQ ID NO: 128 may optionally be provided with lysine (K) removed from the C-terminus.

This ActRIIA-Fc fusion protein is encoded by the following nucleic acid sequence (SEQ ID NO: 129):

| (SEQ ID NO: 129) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGCTATACT TGGTAGATCA GAAACTCAGG |
| 101 | AGTGTCTTTT CTTTAATGCT AATTGGGAAA AAGACAGAAC CAATCAAACT |
| 151 | GGTGTTGAAC CGTGTTATGG TGACAAAGAT AAACGGCGGC ATTGTTTTGC |
| 201 | TACCTGGAAG AATATTTCTG GTTCCATTGA AATAGTGAAA CAAGGTTGTT |
| 251 | GGCTGGATGA TATCAACTGC TATGACAGGA CTGATTGTGT AGAAAAAAAA |
| 301 | GACAGCCCTG AAGTATATTT CTGTTGCTGT GAGGGCAATA TGTGTAATGA |
| 351 | AAAGTTTTCT TATTTTCCGG AGATGGAAGT CACACAGCCC ACTTCAAATC |
| 401 | CAGTTACACC TAAGCCACCC ACCGGTGGTG GAGGTTCTGG AGGTGGAGGA |
| 451 | AGTGGTGGAG GTGGTTCTGG AGGTGGTGGA AGTACTCACA CATGCCCACC |
| 501 | GTGCCCAGCA CCTGAACTCC TGGGGGGACC GTCAGTCTTC CTCTTCCCCC |
| 551 | CAAAACCCAA GGACACCCTC ATGATCTCCC GGACCCCTGA GGTCACATGC |
| 601 | GTGGTGGTGG ACGTGAGCCA CGAAGACCCT GAGGTCAAGT TCAACTGGTA |
| 651 | CGTGGACGGC GTGGAGGTGC ATAATGCCAA GACAAAGCCG CGGGAGGAGC |
| 701 | AGTACAACAG CACGTACCGT GTGGTCAGCG TCCTCACCGT CCTGCACCAG |
| 751 | GACTGGCTGA ATGGCAAGGA GTACAAGTGC AAGGTCTCCA ACAAAGCCCT |
| 801 | CCCAGCCCCC ATCGAGAAAA CCATCTCCAA AGCCAAAGGG CAGCCCCGAG |
| 851 | AACCACAGGT GTACACCCTG CCCCCATCCC GGAAGGAGAT GACCAAGAAC |
| 901 | CAGGTCAGCC TGACCTGCCT GGTCAAAGGC TTCTATCCCA GCGACATCGC |
| 951 | CGTGGAGTGG GAGAGCAATG GGCAGCCGGA GAACAACTAC AAGACCACGC |
| 1001 | CTCCCGTGCT GAAGTCCGAC GGCTCCTTCT TCCTCTATAG CAAGCTCACC |
| 1051 | GTGGACAAGA GCAGGTGGCA GCAGGGGAAC GTCTTCTCAT GCTCCGTGAT |
| 1101 | GCATGAGGCT CTGCACAACC ACTACACGCA GAAGAGCCTC TCCCTGTCTC |
| 1151 | CGGGTAAATG A |

The processed ActRIIA-Fc fusion polypeptide (SEQ ID NO: 130) is as follows, and may optionally be provided with lysine (K) removed from the C-terminus.

| (SEQ ID NO: 130) | |
|---|---|
| 1 | ILGRSETQEC LFFNANWEKD RTNQTGVEPC YGDKDKRRHC FATWKNISGS IEIVKQGCWL |
| 61 | DDINCYDRTD CVEKKDSPEV YFCCCEGNMC NEKFSYFPEM EVTQPTSNPV TPKPPTGGGG |
| 121 | SGGGGSGGGG SGGGGSTHTC PPCPAPELLG GPSVFLFPPK PKDTLMISRT PEVTCVVVDV |
| 181 | SHEDPEVKFN WYVDGVEVHN AKTKPREEQY NSTYRVVSVL TVLHQDWLNG KEYKCKVSNK |
| 241 | ALPAPIEKTI SKAKGQPREP QVYTLPPSRK EMTKNQVSLT CLVKGFYPSD IAVEWESNGQ |
| 301 | PENNYKTTPP VLKSDGSFFL YSKLTVDKSR WQQGNVFSCS VMHEALHNHY TQKSLSLSPG |
| 361 | K |

The complementary form of TβRII-Fc fusion polypeptide (SEQ ID NO: 131) is as follows:

| (SEQ ID NO: 131) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHE PKLPYHDFIL EDAASPKCIM |
| 151 | KEKKKPGETF FMCSCSSDEC NDNIIFSEEY NTSNPDTGGG GSGGGGSGGG |
| 201 | GSGGGGSTHT CPPCPAPELL GGPSVFLFPP KPKDTLMISR TPEVTCVVVD |
| 251 | VSHEDPEVKF NWYVDGVEVH NAKTKPREEQ YNSTYRVVSV LTVLHQDWLN |
| 301 | GKEYKCKVSN KALPAPIEKT ISKAKGQPRE PQVYTLPPSR EEMTKNQVSL |
| 351 | TCLVKGFYPS DIAVEWESNG QPENNYDTTP PVLDSDGSFF LYSDLTVDKS |
| 401 | RWQQGNVFSC SVMHEALHNH YTQKSLSLSP GK |

The leader sequence and linker are underlined. To guide heterodimer formation with the ActRIIA-Fc fusion polypeptide of SEQ ID NOs: 128 and 130 above, two amino acid substitutions (replacing lysines with aspartic acids) can be introduced into the Fc domain of the TβRII-Fc fusion polypeptide as indicated by double underline above. The amino acid sequence of SEQ ID NO: 131 may optionally be provided with lysine (K) added at the C-terminus.

This TβRII-Fc fusion protein is encoded by the following nucleic acid (SEQ ID NO: 132):

| (SEQ ID NO: 132) | |
|---|---|
| 1 | ATGGATGCGA TGAAACGCGG CCTGTGCTGC GTGCTGCTGC TGTGCGGCGC |
| 51 | GGTGTTTGTG AGCCCGGGCG CCACCATTCC GCCGCATGTG CAGAAAAGCG |
| 101 | ATGTGGAAAT GGAAGCGCAG AAAGATGAAA TTATTTGCCC GAGCTGCAAC |
| 151 | CGCACCGCGC ATCCGCTGCG CCATATTAAC AACGATATGA TTGTGACCGA |
| 201 | TAACAACGGC GCGGTGAAAT TTCCGCAGCT GTGCAAATTT TGCGATGTGC |
| 251 | GCTTTAGCAC CTGCGATAAC CAGAAAAGCT GCATGAGCAA CTGCAGCATT |
| 301 | ACCAGCATTT GCGAAAAACC GCAGGAAGTG TGCGTGGCGG TGTGGCGCAA |
| 351 | AAACGATGAA AACATTACCC TGGAAACCGT GTGCCATGAT CCGAAACTGC |
| 401 | CGTATCATGA TTTTATTCTG GAAGATGCGG CGAGCCCGAA ATGCATTATG |
| 451 | AAAGAAAAAA AAAAACCGGG CGAAACCTTT TTTATGTGCA GCTGCAGCAG |
| 501 | CGATGAATGC AACGATAACA TTATTTTTAG CGAAGAATAT AACACCAGCA |
| 551 | ACCCGGATAC CGGTGGCGGC GGCAGCGGCG GCGGCGGCAG CGGCGGCGGC |
| 601 | GGCAGCGGCG GCGGCGGCAG CACCCATACC TGCCCGCCGT GCCCGGCGCC |
| 651 | GGAACTGCTG GGCGGCCCGA GCGTGTTTCT GTTTCCGCCG AAACCGAAAG |
| 701 | ATACCCTGAT GATTAGCCGC ACCCCGGAAG TGACCTGCGT GGTGGTGGAT |
| 751 | GTGAGCCATG AAGATCCGGA AGTGAAATTT AACTGGTATG TGGATGGCGT |
| 801 | GGAAGTGCAT AACGCGAAAA CCAAACCGCG CGAAGAACAG TATAACAGCA |
| 851 | CCTATCGCGT GGTGAGCGTG CTGACCGTGC TGCATCAGGA TTGGCTGAAC |
| 901 | GGCAAAGAAT ATAAATGCAA AGTGAGCAAC AAAGCGCTGC CGGCGCCGAT |
| 951 | TGAAAAAACC ATTAGCAAAG CGAAAGGCCA GCCGCGCGAA CCGCAGGTGT |
| 1001 | ATACCCTGCC GCCGAGCCGC GAAGAAATGA CCAAAAACCA GGTGAGCCTG |
| 1051 | ACCTGCCTGG TGAAAGGCTT TTATCCGAGC GATATTGCGG TGGAATGGGA |
| 1101 | AAGCAACGGC CAGCCGGAAA ACAACTATGA TACCACCCCG CCGGTGCTGG |
| 1151 | ATAGCGATGG CAGCTTTTTT CTGTATAGCG ATCTGACCGT GGATAAAAGC |
| 1201 | CGCTGGCAGC AGGGCAACGT GTTTAGCTGC AGCGTGATGC ATGAAGCGCT |
| 1251 | GCATAACCAT TATACCCAGA AAAGCCTGAG CCTGAGCCCG GGCGATGATG |
| 1301 | ATGATAAAGC GCATCATCAT CATCATCATT AA |

The processed TβRII-Fc fusion protein sequence (SEQ ID NO: 133) is as follows and may optionally be provided with lysine (K) added at the C-terminus.

| (SEQ ID NO: 133) | |
|---|---|
| 1 | TIPPHVQKSD VEMEAQKDEI ICPSCNRTAH PLRHINNDMI VTDNNGAVKF PQLCKFCDVR |
| 61 | FSTCDNQKSC MSNCSITSIC EKPQEVCVAV WRKNDENITL ETVCHDPKLP YHDFILEDAA |
| 121 | SPKCIMKEKK KPGETFFMCS CSSDECNDNI IFSEEYNTSN PDTGGGGSGG GGSGGGGSGG |
| 181 | GGSTHTCPPC PAPELLGGPS VFLFPPKPKD TLMISRTPEV TCVVVDVSHE DPEVKFNWYV |
| 241 | DGVEVHNAKT KPREEQYNST YRVVSVLTVL HQDWLNGKEY KCKVSNKALP APIEKTISKA |
| 301 | KGQPREPQVY TLPPSREEMT KNQVSLTCLV KGFYPSDIAV EWESNGQPEN NYDTTPPVLD |
| 361 | SDGSFFLYSD LTVDKSRWQQ GNVFSCSVMH EALHNHYTQK SLSLSPGK |

The ActRIIA-Fc and TβRII-Fc proteins of SEQ ID NO: 130 and SEQ ID NO: 133, respectively, may be co-expressed and purified from a CHO cell line, to give rise to a heteromeric complex comprising ActRIIA-Fc:TβRII-Fc.

In another approach to promote the formation of heteromultimer complexes using asymmetric Fc fusion proteins the Fc domains are altered to introduce complementary hydrophobic interactions and an additional intermolecular disulfide bond as illustrated in the ActRIIA-Fc and TβRII-Fc polypeptide sequences of SEQ ID NOs: 134, 136, 137, and 139, respectively. The ActRIIA-Fc fusion polypeptide and TβRII-Fc fusion polypeptide each employ the tissue plasminogen activator (TPA) leader.

The ActRIIA-Fc polypeptide sequence (SEQ ID NO: 134) is shown below:

| (SEQ ID NO: 134) | |
|---|---|
| 1 | MDAMKRGLCC VLLLCGAVFV SPGAAILGRS ETQECLFFNA NWEKDRTNQT |
| 51 | GVEPCYGDKD KRRHCFATWK NISGSIEIVK QGCWLDDINC YDRTDCVEKK |
| 101 | DSPEVYFCCC EGNMCNEKFS YFPEMEVTQP TSNPVTPKPP TGGGGSGGGG |
| 151 | SGGGGSGGGG STHTCPPCPA PELLGGPSVF LFPPKPKDTL MISRTPEVTC |
| 201 | VVVDVSHEDP EVKFNWYVDG VEVHNAKTKP REEQYNSTYR VVSVLTVLHQ |
| 251 | DWLNGKEYKC KVSNKALPAP IEKTISKAKG QPREPQVYTL PPCREEMTKN |
| 301 | QVSLWCLVKG FYPSDIAVEW ESNGQPENNY KTTPPVLDSD GSFFLYSKLT |
| 351 | VDKSRWQQGN VFSCSVMHEA LHNHYTQKSL SLSPGK |

The leader (signal) sequence and linker are underlined. To promote formation of the ActRIIA-Fc:TβRII-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 134 may optionally be provided with lysine (K) removed from the C-terminus.

This ActRIIA-Fc fusion protein is encoded by the following nucleic acid sequence (SEQ ID NO: 135):

| (SEQ ID NO: 135) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGCTATACT TGGTAGATCA GAAACTCAGG |
| 101 | AGTGTCTTTT CTTTAATGCT AATTGGGAAA AAGACAGAAC CAATCAAACT |
| 151 | GGTGTTGAAC CGTGTTATGG TGACAAAGAT AAACGGCGGC ATTGTTTTGC |
| 201 | TACCTGGAAG AATATTTCTG GTTCCATTGA AATAGTGAAA CAAGGTTGTT |
| 251 | GGCTGGATGA TATCAACTGC TATGACAGGA CTGATTGTGT AGAAAAAAAA |
| 301 | GACAGCCCTG AAGTATATTT CTGTTGCTGT GAGGGCAATA TGTGTAATGA |
| 351 | AAAGTTTTCT TATTTTCCGG AGATGGAAGT CACACAGCCC ACTTCAAATC |
| 401 | CAGTTACACC TAAGCCACCC ACCGGTGGTG GAGGTTCTGG AGGTGGAGGA |
| 451 | AGTGGTGGAG GTGGTTCTGG AGGTGGTGGA AGTACTCACA CATGCCCACC |
| 501 | GTGCCCAGCA CCTGAACTCC TGGGGGGACC GTCAGTCTTC CTCTTCCCCC |
| 551 | CAAAACCCAA GGACACCCTC ATGATCTCCC GGACCCCTGA GGTCACATGC |
| 601 | GTGGTGGTGG ACGTGAGCCA CGAAGACCCT GAGGTCAAGT TCAACTGGTA |
| 651 | CGTGGACGGC GTGGAGGTGC ATAATGCCAA GACAAAGCCG CGGGAGGAGC |
| 701 | AGTACAACAG CACGTACCGT GTGGTCAGCG TCCTCACCGT CCTGCACCAG |
| 751 | GACTGGCTGA ATGGCAAGGA GTACAAGTGC AAGGTCTCCA ACAAAGCCCT |
| 801 | CCCAGCCCCC ATCGAGAAAA CCATCTCCAA AGCCAAAGGG CAGCCCCGAG |
| 851 | AACCACAGGT GTACACCCTG CCCCCATGCC GGGAGGAGAT GACCAAGAAC |
| 901 | CAGGTCAGCC TGTGGTGCCT GGTCAAAGGC TTCTATCCCA GCGACATCGC |
| 951 | CGTGGAGTGG GAGAGCAATG GGCAGCCGGA GAACAACTAC AAGACCACGC |
| 1001 | CTCCCGTGCT GGACTCCGAC GGCTCCTTCT TCCTCTATAG CAAGCTCACC |
| 1051 | GTGGACAAGA GCAGGTGGCA GCAGGGGAAC GTCTTCTCAT GCTCCGTGAT |
| 1101 | GCATGAGGCT CTGCACAACC ACTACACGCA GAAGAGCCTC TCCCTGTCTC |
| 1151 | CGGGTAAATG A |

The processed ActRIIA-Fc fusion polypeptide is as follows:

| (SEQ ID NO: 136) | |
|---|---|
| 1 | ILGRSETQEC LFFNANWEKD RTNQTGVEPC YGDKDKRRHC FATWKNISGS IEIVKQGCWL |
| 61 | DDINCYDRTD CVEKKDSPEV YFCCCEGNMC NEKFSYFPEM EVTQPTSNPV TPKPPTGGGG |
| 121 | SGGGGSGGGG SGGGGSTHTC PPCPAPELLG GPSVFLFPPK PKDTLMISRT PEVTCVVVDV |
| 181 | SHEDPEVKFN WYVDGVEVHN AKTKPREEQY NSTYRVVSVL TVLHQDWLNG KEYKCKVSNK |
| 241 | ALPAPIEKTI SKAKGQPREP QVYTLPPCRE EMTKNQVSLW CLVKGFYPSD IAVEWESNGQ |
| 301 | PENNYKTTPP VLDSDGSFFL YSKLTVDKSR WQQGNVFSCS VMHEALHNHY TQKSLSLSPG |
| 361 | K |

The complementary form of TβRII-Fc fusion polypeptide (SEQ ID NO: 137) is as follows and may optionally be provided with lysine (K) removed from the C-terminus.

| (SEQ ID NO: 137) | |
|---|---|
| 1 | MDAMKRGLCC VLLLCGAVFV SPGATIPPHV QKSDVEMEAQ KDEITCPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSTHT CPPCPAPELL GGPSVFLFPP KPKDTLMISR TPEVTCVVVD |
| 251 | VSHEDPEVKF NWYVDGVEVH NAKTKPREEQ YNSTYRVVSV LTVLHQDWLN |
| 301 | GKEYKCKVSN KALPAPIEKT ISKAKGQPRE PQVCTLPPSR EEMTKNQVSL |
| 351 | SCAVKGFYPS DIAVEWESNG QPENNYKTTP PVLDSDGSFF LVSKLTVDKS |
| 401 | RWQQGNVFSC SVMHEALHNH YTQKSLSLSP GK |

The leader sequence and the linker are underlined. To guide heterodimer formation with the ActRIIA-Fc fusion polypeptide of SEQ ID NOs: 134 and 136 above, four amino acid substitutions can be introduced into the Fc domain of the TβRII fusion polypeptide as indicated by double underline above. The amino acid sequence of SEQ ID NO: 137 may optionally be provided with lysine (K) removed from the C-terminus.

This A TβRII-Fc fusion protein is encoded by the following nucleic acid sequence (SEQ ID NO: 138):

| (SEQ ID NO: 138) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA GGTTCTGGAG GTGGAGGAAG TGGTGGAGGT |
| 601 | GGTTCTGGAG GTGGTGGAAG TACTCACACA TGCCCACCGT GCCCAGCACC |
| 651 | TGAACTCCTG GGGGGACCGT CAGTCTTCCT CTTCCCCCCA AAACCCAAGG |
| 701 | ACACCCTCAT GATCTCCCGG ACCCCTGAGG TCACATGCGT GGTGGTGGAC |
| 751 | GTGAGCCACG AAGACCCTGA GGTCAAGTTC AACTGGTACG TGGACGGCGT |
| 801 | GGAGGTGCAT AATGCCAAGA CAAAGCCGCG GGAGGAGCAG TACAACAGCA |
| 851 | CGTACCGTGT GGTCAGCGTC CTCACCGTCC TGCACCAGGA CTGGCTGAAT |
| 901 | GGCAAGGAGT ACAAGTGCAA GGTCTCCAAC AAAGCCCTCC CAGCCCCCAT |
| 951 | CGAGAAAACC ATCTCCAAAG CCAAAGGGCA GCCCCGAGAA CCACAGGTGT |
| 1001 | GCACCCTGCC CCCATCCCGG GAGGAGATGA CCAAGAACCA GGTCAGCCTG |
| 1051 | TCCTGCGCCG TCAAAGGCTT CTATCCCAGC GACATCGCCG TGGAGTGGGA |
| 1101 | GAGCAATGGG CAGCCGGAGA ACAACTACAA GACCACGCCT CCCGTGCTGG |
| 1151 | ACTCCGACGG CTCCTTCTTC CTCGTGAGCA AGCTCACCGT GGACAAGAGC |
| 1201 | AGGTGGCAGC AGGGGAACGT CTTCTCATGC TCCGTGATGC ATGAGGCTCT |
| 1251 | GCACAACCAC TACACGCAGA AGAGCCTCTC CCTGTCTCCG GGTAAATGA |

A processed TβRII-Fc fusion protein sequence is as follows and may optionally be provided with lysine (K) removed from the C-terminus.

| (SEQ ID NO: 139) | |
|---|---|
| 1 | TIPPHVQKSD VEMEAQKDEI ICPSCNRTAH PLRHINNDMI VTDNNKAVKF PQLCKFCDVR |
| 61 | FSTCDNQKSC MSNCSITSIC EKPQEVCVAV WRKNDENITL ETVCHDPKLP YHDFILEDAA |
| 121 | SPKCIMKEKK KPGETFFMCS CSSDECNDNI IFSEEYNTSN PDTGGGGSGG GGSGGGGSGG |
| 181 | GGSTHTCPPC PAPELLGGPS VFLFPPKPKD TLMISRTPEV TCVVVDVSHE DPEVKFNWYV |
| 241 | DGVEVHNAKT KPREEQYNST YRVVSVLTVL HQDWLNGKEY KCKVSNKALP APIEKTISKA |
| 301 | KGQPREPQVC TLPPSREEMT KNQVSLSCAV KGFYPSDIAV EWESNGQPEN NYKTTPPVLD |
| 361 | SDGSFFLVSK LTVDKSRWQQ GNVFSCSVMH ELAHNHYTQK SLSLSPGK |

ActRIIA-Fc and TβRII-Fc proteins of SEQ ID NO: 134 and SEQ ID NO: 137, respectively, may be co-expressed and purified from a CHO cell line, to give rise to a heteromeric complex comprising ActRIIA-Fc:TβRII-Fc.

In order to compare the activity of the ActRIIA-Fc:TβRII-Fc heterodimers, ActRIIA-Fc and TβRII-Fc homodimers were generated, which each comprise either the ActRIIA or TβRII extracellular domains as present in any one of SEQ ID NO: 128, 130, 131, 133, 134, 136, 137, and 139; an unmodified hG1Fc domain (promotes homodimer formation); and a (G4S)4 linker positioned between the ActRIIA or TβRII extracellular portion and the unmodified Fc portion. Both of these homodimers were expressed using the TPA leader sequence of SEQ ID NO: 23.

Purification of various heterodimer and homodimers described above could be achieved by a series of column chromatography steps, including, for example, three or more of the following, in any order: protein A chromatography, Q sepharose chromatography, phenylsepharose chromatography, size exclusion chromatography and epitope-based affinity chromatography (e.g., with an antibody or functionally equivalent ligand directed against an epitope on TβRII or ActRIIA), and multimodal chromatography (e.g., with resin containing both electrostatic and hydrophobic ligands). The purification could be completed with viral filtration and buffer exchange.

### Example 6. Differential Ligand Inhibition by Receptor Fusion Protein Variants in Cell-Based Assay

A reporter gene assay in A549 cells was used to determine the ability of an ActRIIA-Fc:TβRII-Fc heterodimer to inhibit activity of TGFβ1, TGFβ2, TGFβ3, activin A, activin B, GDF11, and BMP10 and compared to the inhibiting activity of an ActRIIA-Fc homodimer and TβRII-fc homodimer, which are all described above in Example 3. This assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates. On the second day, a solution containing pGL3(CAGA)12, pRLCMV, X-tremeGENE 9 (Roche Applied Science), and OptiMEM (Invitrogen) was preincubated, then added to Eagle's minimum essential medium (EMEM, ATCC®) supplemented with 0.1% BSA, which was applied to the plated cells for incubation overnight at 37° C., 5% CO₂. On the third day, medium was removed, and cells were incubated overnight at 37° C., 5% CO₂ with a mixture of ligands and inhibitors prepared as described below.

Serial dilutions of test articles were made in a 48-well plate in assay buffer (EMEM+0.1% BSA). An equal volume of assay buffer containing the test ligand was added to obtain a final ligand concentration equal to the EC50 determined previously. Test solutions were incubated at 37° C. for 30 minutes, then a portion of the mixture was added to all wells. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred in duplicate to a chemiluminescence plate (96-well) and analyzed in a luminometer with reagents from a Dual-Luciferase Reporter Assay system (Promega E1980) to determine normalized luciferase activity.

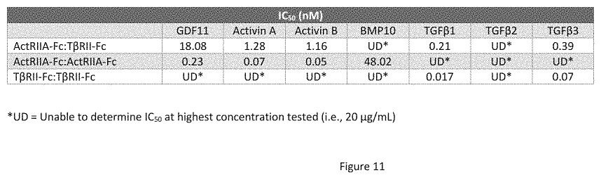

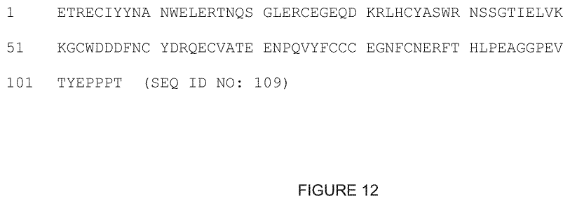

As illustrated in FIG. 11, the TβRII-Fc homodimer was capable of inhibiting TGFβ1 and TGFβ33 in this cell-based assay but did not inhibit TGFβ2, activin A, activin B, GDF11, or BMP10. In contrast, the ActRIIA-Fc homodimer was capable of inhibiting activin A, activin B, GDF11, and BMP10 but did not inhibit TGFβ1, TGFβ2, or TGFβ3. The ActRIIA-Fc:TβRII-Fc heterodimer was capable of inhibiting TGFβ1, TGFβ3, activin A, activin B, and GDF11 and but did not inhibit BMP10 or TGFβ2. These data demonstrate that ActRIIA-ft:TβRII-fc heterodimers retain many of potent inhibitor characteristics of ActRIIA-Fc and homodimers and thus represent an interesting class of ligand traps that are uniquely capable of affecting two distinct groups of Smad 2/3-related TGFβ superfamily ligands (i.e., the TGFβs and activin/GDFs). Moreover, the ActRIIA-Fc:TβRII-Fc heterodimer did not inhibit BMP910, and thus with respect to ActRIIA-associated ligands, ActRIIA-Fc:TβRII-Fc is a more selective antagonist than an ActRIIA homodimer. Accordingly, ActRIIA-Fc:TβRII-Fc heterodimers will be more useful than ActRIIA homodimer in certain applications where such selective antagonism is desired in combination with inhibition of TGFβ1 and TGFβ3.

### Example 7: Generation of an ActRIIA-Fc Fusion Protein

An ActRIIA fusion protein that has the extracellular domain of human ActRIIA fused to a human or mouse Fc domain with a linker in between was generated. The constructs are referred to as hActRIIA-hFc and hActRIIA-mFc, respectively.

ActRIIA-hFc is shown below as purified from CHO
cell lines (SEQ ID NO: 140):
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPPTGGGTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK

The ActRIIA-hFc and ActRIIA-mFc proteins were expressed in CHO cell lines. Three different leader sequences were considered:

(i) Honey bee mellitin (SEQ ID NO: 24)

(ii) Tissue plasminogen activator (SEQ ID NO: 23)

(iii) Native ActRIIA leader sequence: MGAAAKLAFAVFLISCSSGA (SEQ ID NO: 143).

The selected form employs the TPA leader and has the following unprocessed amino acid sequence:

(SEQ ID NO: 141)
MDAMKRGLCCVLLLCGAVFVSPGAAILGRSETQECLFFNANWEKDRTNQT
GVEPCYGDKDKRRHCFATWKNISGSIEIVKQGCWLDDINCYDRTDCVEKK
DSPEVYFCCCEGNMCNEKFSYFPEMEVTQPTSNPVTPKPPTGGGTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PVPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK

This polypeptide is encoded by the following nucleic acid sequence:

(SEQ ID NO: 142)
ATGGATGCAATGAAGAGAGGGCTCTGCTGTGTGCTGCTGCTGTGTGGAGC
AGTCTTCGTTTCGCCCGGCGCCGCTATACTTGGTAGATCAGAAACTCAGG
AGTGTCTTTTTTTAATGCTAATTGGGAAAAAGACAGAACCAATCAAACTG
GTGTTGAACCGTGTTATGGTGACAAAGATAAACGGCGGCATTGTTTTGCT
ACCTGGAAGAATATTTCTGGTTCCATTGAATAGTGAAACAAGGTTGTTGG
CTGGATGATATCAACTGCTATGACAGGACTGATTGTGTAGAAAAAAAAGA
CAGCCCTGAAGTATATTTCTGTTGCTGTGAGGGCAATATGTGTAATGAAA
AGTTTTCTTATTTTCCGGAGATGGAAGTCACACAGCCCACTTCAAATCCA
GTTACACCTAAGCCACCCACCGGTGGTGGAACTCACACATGCCCACCGTG
CCCAGCACCTGAACTCCTGGGGGGACCGTCAGTCTTCCTCTTCCCCCCAA
AACCCAAGGACACCCTCATGATCTCCCGGACCCCTGAGGTCACATGCGTG
GTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGT
ACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCC
AGTCCCCATCGAGAAAACCATCTCCAAAGCCAAAGGGCAGCCCCGAGAAC
CACAGGTGTACACCCTGCCCCCATCCCGGGAGGAGATGACCAAGAACCAG
GTCAGCCTGACCTGCCTGGTCAAAGGCTTCTATCCCAGCGACATCGCCGT
GGAGTGGGAGAGCAATGGGCAGCCGGAGAACAACTACAAGACCACGCCTC
CCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCA
TGAGGCTCTGCACAACCACTACACGCAGAAGAGCCTCTCCCTGTCTCCGG
GTAAATGAGAATTC

Both ActRIIA-hFc and ActRIIA-mFc were remarkably amenable to recombinant expression. The protein was purified as a single, well-defined peak of protein. N-terminal sequencing revealed a single sequence of -ILGRSETQE (SEQ ID NO: 144). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

ActRIIA-hFc and ActRIIA-mFc showed a high affinity for ligands. GDF-11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 8: Generation of an ActRIIB-Fc Fusion Protein

A soluble ActRIIB fusion protein that has the extracellular domain of human ActRIIB fused to a human or mouse Fc domain with a linker in between was constructed. The constructs are referred to as hActRIIB-hFc and hActRIIB-mFc, respectively.

ActRIIB-hFc is shown below as purified from CHO cell lines (SEQ ID NO: 145):

GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPTGGGTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPVPIEKTISKAKGQPREPQVYTLPPS
REEMTKNQVSLTCLVKGFYPSDIAVEWESNGQPENNYKTTPPVLDSDGSF
FLYSKLTVDKSRWQQGNVFSCSVMHEALHNHYTQKSLSLSPGK

The ActRIIB-hFc and ActRIIB-mFc proteins were expressed in CHO cell lines. Three different leader sequences were considered: (i) Honey bee mellitin (SEQ ID NO: 24), ii) Tissue plasminogen activator (SEQ ID NO: 23), and (iii) Native hActRIIB: MGAAAKLAFAVFLISCSSGA (SEQ ID NO: 148).

The selected form employs the TPA leader and has the following unprocessed amino acid sequence (SEQ ID NO: 146):

MDAMKRGLCCVLLLCGAVFVSPGASGRGEAETRECIYYNANWELERTNQS
GLERCEGEQDKRLHCYASWRNSSGTIELVKKGCWLDDFNCYDRQECVATE
ENPQVYFCCCEGNFCNERFTHLPEAGGPEVTYEPPPTAPTGGGTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
VPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK

This polypeptide is encoded by the following nucleic acid sequence (SEQ ID NO: 147):

A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GCTAGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

N-terminal sequencing of the CHO-cell-produced material revealed a major sequence of -GRGEAE (SEQ ID NO: 149). Notably, other constructs reported in the literature begin with an -SGR . . . sequence.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

Affinities of several ligands for ActRIIB(20-134)-hFc were evaluated in vitro with a Biacore™ instrument, and the results are summarized in the table below. ActRIIB(20-134)-hFc bound activin A, activin B, and GDF11 with high affinity.

### Ligand Affinities of ActRIIB-hFc Forms:

| | Activin A | Activin B | GDF11 |
|---|---|---|---|
| Fusion Construct | (e−11) | (e−11) | (e−11) |
| ActRIIB(20-134)-hFc | 1.6 | 1.2 | 3.6 |

### Example 9: Antitumor Activity of Activin and TGFβ Antagonists

Potential antitumor activity of hActRIIA-mFc, hActRIIB-mFc, and hTβRII-mFc fusion proteins were investigated in a syngeneic murine leukemia model. Eight-week-old BALB/c mice were randomly assigned to treatment (n=10 per group) and treated intraperitoneally with hActRIIA-mFc (10 mg/kg), hActRIIB-mFc (10 mg/kg), hTβRII_{long}(23-184)-mFc (10 mg/kg), or vehicle (phosphate-buffered saline, PBS, 5 ml/kg) twice weekly beginning two days prior to administration of cancer cells. On day 0, each mouse was inoculated subcutaneously with 1×10⁶ RL♂1 (RLmale1) cells suspended in PBS (100 μL). RLmale1 is an x-ray-induced leukemia of BALB/c origin (Sato H et al., 1973, J Exp Med 138:593-606). After inoculation of mice, body weight and tumor volume were measured twice weekly. Tumor volumes were calculated from two-dimensional measurements obtained with calipers: tumor volume (in mm³)=(L×W×W)/2 where L and W are the tumor length and width (in mm), respectively. Complete tumor regression and tumor-free survival were both defined according to Teicher B A (ed) Anticancer Drug Development Guide: Preclinical Screening, Clinical Trials, and Approval; Humana Press, 1997. Per local IACUC regulations, endpoints used for survival analysis were a tumor volume larger than 2000 mm³, loss of body weight greater than 20%, or hind-leg paralysis. The survival curves of different groups were compared by median survival as well as by log-rank (Mantel-Cox) test.

As shown in the following table, both hActRIIA-mFc and hActRIIB-mFc exhibited antitumor activity. However, hTβRII-mFc did not demonstrate any appreciable antitumor activity in this model.

| | | | | | | % tumor | Median |
|---|---|---|---|---|---|---|---|
| Test | | | Dose | | Sched- | free | survival |
| article | Strain | n | (mg/kg) | Route | ule | (day 56) | (days) |
| Vehicle | BALB/c | 10 | — | i.p. | biw | 0 | 15 |
| hActRIIA- | BALB/c | 10 | 10 | i.p. | biw | 20 | 21.5 |
| mFc | | | | | | | |
| hActRIIB- | BALB/c | 10 | 10 | i.p. | biw | 20 | 32.5 |
| mFc | | | | | | | |
| hTβRII- | BALB/c | 10 | 10 | i.p. | biw | 0 | 17 |
| mFc | | | | | | | |

Treatment with hActRIIA-mFc or hActRIIB-mFc led to 2 of 10 mice (20%) with tumor-free status on day 56, compared to none of the vehicle and hTβRII-mFc treated mice. Increased median survival and high significance in the log-rank test also indicate that hActRIIA-mFc and hActRIIB-mFc each increased survival of tumor-bearing mice. The initial response to ActRIIB-mFc was particularly robust, as 50% of hActRIIB-mFc-treated mice showed complete tumor regression by day 34 compared to none in the vehicle-treated group. These results show that hActRIIA-mFc and hActRIIB-mFc possess antitumor activity in vivo, indicating that these proteins, as well as other activin antagonists, may be useful in the treatment of cancer.

Using the same murine leukemia model, it was then assessed whether hActRIIB-hFc has antitumor activity similar to that of hActRIIB-mFc and whether antitumor activity is dependent on T cell-mediated immunity. Eight-week-old BALB/c mice were randomly assigned to treatment (n=10 per group) and treated intraperitoneally with hActRIIB-mFc (10 mg/kg), hActRIIB-hFc (10 mg/kg), or vehicle (PBS, 5 ml/kg) twice weekly beginning two days prior to administration of cancer cells. In addition, 7-week-old NCr-nude mice with defective T cell immunity were randomly assigned to treatment (n=10 per group) and treated intraperitoneally with hActRIIB-mFc (10 mg/kg), hActRIIB-hFc (10 mg/kg), or vehicle (PBS, 5 ml/kg) twice weekly beginning two days prior to administration of cancer cells. Finally, the four mice that had remained tumor free for approximately 7 weeks during the experiment described above (two mice treated with hActRIIA-mFc and two mice treated with hActRIIB-mFc) were re-challenged with RLmale1 cells to test for antitumor immune memory. On day 0, each mouse was inoculated subcutaneously with 1×10⁶ RL♂1 (RLmale1) cells suspended in PBS (100 μL). After mouse inoculation, body weight and tumor volume were measured twice weekly. Per local IACUC regulations, endpoints used for survival analysis were a tumor volume larger than 2000 mm³, loss of body weight greater than 20%, or hind-leg paralysis.

As show in the table below, antitumor effects of hActRIIB-mFc and hActRIIB-hFc were dependent on mouse strain.

| | | | | | | % tumor | Median | Log- |
|---|---|---|---|---|---|---|---|---|
| Test | | | Dose | | | free | survival | rank test |
| article | Strain | n | (mg/kg) | Route | Schedule | (day 56) | (days) | (P value) |
| Vehicle | BALB/c | 10 | — | i.p. | biw | 0 | 17 | — |
| hActRIIB- | BALB/c | 10 | 10 | i.p. | biw | 10 | 36 | 0.002 |
| mFc | | | | | | | | |
| hActRIIB- | BALB/c | 10 | 10 | i.p. | biw | 30 | 27.5 | 0.003 |
| hFc | | | | | | | | |
| Vehicle | NCr- | 10 | — | i.p. | biw | 0 | 12 | — |
| | nudc | | | | | | | |
| hActRIIB- | NCr- | 10 | 10 | i.p. | biw | 0 | 12 | 0.07 |
| mFc | nudc | | | | | | | |
| hActRIIB- | NCr- | 10 | 10 | i.p. | biw | 0 | 12 | 0.03 |
| hFc | nude | | | | | | | |
| hActRIIA- | BALB/c | 2 | — | — | — | 100 | — | — |
| mFc | | | | | | | | |
| hActRIIB- | BALB/c | 2 | — | — | — | 100 | — | — |
| mFc | | | | | | | | |

Both hActRIIB-hFc and hActRIIB-mFc exhibited antitumor activity in immunocompetent BALB/c mice, as shown in the following table. Treatment with hActRIIB-mFc or hActRIIB-hFc led to 10% or 30% of mice, respectively, with tumor-free status on day 56, compared to none of the vehicle-treated mice. Increased median survival and high significance in the log-rank test also demonstrate that hActRIIB-mFc and hActRIIB-hFc each promoted survival of tumor-bearing mice. Importantly, the antitumor effects of hActRIIB-mFc and hActRIIB-hFc in NCr-nude mice were absent or markedly blunted compared to BALB/c mice, thereby implicating T cell immunity in the mechanism of action for these inhibitors of ActRIIB ligands. Moreover, all four tumor-free mice carried over from the previous experiment exhibited no detectable tumor growth throughout the present experiment despite a repeat inoculation with RLmale1 tumor cells. These results provide further evidence that immune cells mediate the regression of RLmale1 tumors caused by treatment with hActRIIA-mFc or hActRIIB-mFc on a BALB/c background and that the effective antitumor immune response generated immunologic memory to tumor antigens. Furthermore, these results confirm antitumor activity of hActRIIB-hFc and hActRIIB-mFc in vivo and strongly implicate T cell immunity in this activity. Together, the data suggest that activin antagonists may be used to potentiate immune activity in vivo and thus such antagonists may be useful in treating a variety of disorders and conditions wherein increased immune activity is desirable (e.g., immune-oncology applications as well as treatment of a variety of pathogens). While not wishing to be bound by any particular theory, it is believed that such activin antagonist may be particularly useful in treating cancer when used in combination with an immune checkpoint antagonist (e.g., an antibody, or antigen-binding fragment thereof, that binds and inhibits one or more of (e.g., PD-1, PD-L1, CTLA4, BTLA, LAG3, TIM3, LAIR1, B7-DC, HVEM, TIM4, B7-H3, and/or B7-H4).

### Example 10: Antitumor Activity of Activin and TGFβ Antagonists Combination Therapy

Using the same murine leukemia model as described in Example 9, it was then investigated whether hActRIIB-hFc antitumor activity can be enhanced by combining it with a TGFβ antagonist. For the combination study, a pan-specific TGFβ antibody (one that binds to TGFβ1, TGFβ2, and TGFβ3 with high affinity) was used as the TGFβ antagonist.

Eight-week-old BALB/c mice were randomly assigned to treatment (n=10 per group) and treated intraperitoneally with hActRIIB-hFc (10 mg/kg), TGFβ antibody (mAb) (10 mg/kg), combination of hActRIIA-hFc and TGFβ mAb (both at 10 mg/kg), or vehicle (phosphate-buffered saline, PBS, 5 ml/kg) twice weekly beginning two days prior to administration of cancer cells. On day 0, each mouse was inoculated subcutaneously with 1×10⁶ RL♂1 (RLmale1) cells suspended in PBS (100 μL). RLmale1 is an x-ray-induced leukemia of BALB/c origin (Sato H et al., 1973, J Exp Med 138:593-606). After inoculation of mice, body weight and tumor volume were measured twice weekly as described in the previous example. The survival curves of different groups were compared by median survival as well as by log-rank (Mantel-Cox) test.

As shown in the following table, combination therapy with an activin antagonist and a TGFβ antagonist exhibited greater antitumor activity than observed for each antagonist alone.

| | | | | | | % tumor | Median |
|---|---|---|---|---|---|---|---|
| Test | | | Dose | | Sched- | free | survival |
| article | Strain | n | (mg/kg) | Route | ule | (day 56) | (days) |
| Vehicle | BALB/c | 10 | — | i.p. | biw | 0 | 15 |
| hActRIIB- | BALB/c | 10 | 10 | i.p. | biw | 30 | 17 |
| hFc | | | | | | | |
| TGFβ | BALB/c | 10 | 10 | i.p. | biw | 20 | 15 |
| mAb | | | | | | | |
| Combo | BALB/c | 10 | 10 (of | i.p. | biw | 70 | >31 |
| | | | each | | | | |
| | | | agent) | | | | |

Treatment with hActRIIB-hFc alone or TGFβ mAb alone showed modest effects on tumor regression in this model, 30% and 20% tumor-free status respectively. Combined treatment with hActRIIB-hFc and TGFβ mAb led to a surprising and significant increase in antitumor activity, 70% tumor-free status and approximately doubled the median survival time. Synergy of this type is generally considered evidence that the individual agents are acting through different cellular mechanism. Therefore, while inhibition of either the activin or TGFβ signaling pathway may promote antitumor activity, inhibition of both pathways may be used to synergistically increase antitumor activity in such experimental or clinical situations where increased antitumor activity is desirable. Together, these data indicate that activin and TGFβ antagonists can be used alone but particularly in combination to treat cancer. While not wishing to be bound by any particular theory, it is believed that such activin antagonist and TGFβ antagonists, alone or in combination, may be particularly useful in treating cancer when used in combination with an immune checkpoint antagonist (e.g., an antibody, or antigen-binding fragment thereof, that binds and inhibits one or more of (e.g., PD-1, PD-L1, CTLA4, BTLA, LAG3, TIM3, LAIR1, B7-DC, HVEM, TIM4, B7-H3, and/or B7-H4).

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

We claim:

1. A fusion protein comprising two or more domains selected from an activin antagonist domain, a TGFβ antagonist domain, and an immune checkpoint antagonist domain.

2. The fusion protein of claim 1, wherein the protein comprises an activin antagonist domain and a TGFβ antagonist domain.

3. The fusion protein of claim 1, wherein the protein comprises an activin antagonist domain and an immune checkpoint antagonist domain.

4. The fusion protein of claim 1 wherein the protein comprises a TGFβ antagonist domain and an immune checkpoint antagonist domain.

5. The fusion protein of claim 1, wherein the protein comprises an activin antagonist domain, a TGFβ antagonist domain, and an immune checkpoint antagonist domain.

6. The fusion protein of any preceding claim, wherein the fusion protein further comprises a polypeptide domain that is heterologous to the activin antagonist domain, TGFβ antagonist domain, and/or immune checkpoint antagonist domain.

7. The fusion protein of any preceding claim, wherein the fusion protein further comprises a linker domain.

8. The fusion protein of any preceding claim, wherein the domains of the fusion protein are arranged in an order selected from the group consisting of:
a. A-X-T;
b. A-X-I;
c. T-X-I;
d. A-X-H-X-T;
e. A-X-H-X-I;
f. I-X-H-X-T;
g. A-X-T-X-I;
h. T-X-A-X-I;
i. A-X-I-X-T;
j. A-X-T-H-X-I;
k. T-X-A-H-X-I;
l. A-X-I-H-X-T;
m. A-X-H-T-X-I;
n. T-X-H-A-X-I; and
o. A-X-H-I-X-T,
wherein (i) “A” corresponds to an activin antagonist domain; (ii) “T” corresponds to a TGFβ antagonist domain; (iii) “I” corresponds to an immune checkpoint antagonist domain; (iv) “H” corresponds to a polypeptide domain that is heterologous to the activin antagonist domain, TGFβ antagonist domain, and immune checkpoint antagonist domains; and (v) “X” corresponds to an optional linker domain; and wherein the arrangement of the domains is either N-terminus to C-terminus or C-terminus to N-terminus.

9. A homodimer comprising the fusion protein of any of the preceding claims.

10. A heterodimer comprising two or more polypeptide domains selected from an activin antagonist domain, a TGFβ antagonist domain, and an immune checkpoint antagonist domain.

11. The heterodimer of claim 10, wherein the heterodimer comprises two polypeptides selected from:
a. A-X-T;
b. A-X-I;
c. T-X-I;
d. A-X-H-X-T;
e. A-X-H-X-I;
f. I-X-H-X-T;
g. A-X-T-X-I;
h. T-X-A-X-I;
i. A-X-I-X-T;
j. A-X-T-H-X-I;
k. T-X-A-H-X-I;
l. A-X-I-H-X-T;
m. A-X-H-T-X-I;
n. T-X-H-A-X-I; and
o. A-X-H-I-X-T,
wherein (i) “A” corresponds to an activin antagonist domain; (ii) “T” corresponds to a TGFβ antagonist domain; (iii) “I” corresponds to an immune checkpoint antagonist domain; (iv) “H” corresponds to a polypeptide domain that is heterologous to the activin antagonist domain, TGFβ antagonist domain, and immune checkpoint antagonist domains; and (v) “X” corresponds to an optional linker domain; and wherein the arrangement of the domains is either N-terminus to C-terminus or C-terminus to N-terminus.

12. A heterodimer comprising:
a. a first polypeptide comprising an immune checkpoint antagonist domain and a TGFβ antagonist domain; and
b. a second polypeptide comprising an activin antagonist domain.

13. The heterodimer of claim 12, wherein the second polypeptide further comprises an immune checkpoint antagonist domain.

14. A heterodimer comprising:
a. a first polypeptide comprising a TGFβ antagonist domain and an activin antagonist domain; and
b. a second polypeptide comprising an immune checkpoint antagonist domain.

15. The heterodimer of claim 14, wherein the second further comprise a TGFβ antagonist domain.

16. The heterodimer of claim 14, wherein the second further comprise an activin antagonist domain.

17. The heterodimer of any one of claims 12-16, wherein the heterodimer further comprises one or more polypeptide domains that are heterologous to the activin antagonist domain, TGFβ antagonist domain, and/or immune checkpoint antagonist domain.

18. The heterodimer of any preceding claim, wherein the heterodimer further comprises one or more linker domains.

19. The fusion protein, homodimer or heterodimer of any preceding claim, wherein the activin antagonist domain is an ActRIIA polypeptide.

20. The fusion protein, homodimer or heterodimer of claim 19, wherein the ActRIIA polypeptide is selected from the group consisting of:
a. a polypeptide comprising an amino acid sequence that is at least 75% identical to a sequence beginning at any one of positions 21 to 30 of SEQ ID NO: 110, and ending at any one of positions 110 to 135 of SEQ ID NO: 110;
b. a polypeptide comprising an amino acid sequence that is at least 75% identical to a sequence beginning at position 21 of SEQ ID NO: 110, and ending at position 135 of SEQ ID NO: 110;
c. a polypeptide comprising an amino acid sequence that is at least 75% identical to a sequence beginning at position 30 of SEQ ID NO: 110, and ending at position 110 of SEQ ID NO: 110;
d. a polypeptide comprising an amino acid sequence that is at least 75% identical to SEQ ID NO: 111; and
e. a polypeptide comprising an amino acid sequence that is at least 75% identical to SEQ ID NO: 112.

21. The fusion protein, homodimer or heterodimer of claim 19 or 20, wherein the ActRIIB polypeptide binds to activin.

22. The fusion protein, homodimer or heterodimer of claim 21, wherein the ActRIIB polypeptide binds to activin A.

23. The fusion protein, homodimer or heterodimer of claim 21 or 22, wherein the ActRIIB polypeptide binds to activin B.

24. The fusion protein, homodimer or heterodimer of any one claims 21-24, wherein the ActRIIB polypeptide further binds to GDF8 and/or GDF11.

25. The fusion protein, homodimer or heterodimer of any one of claims 21-24, wherein the ActRIIB polypeptide inhibits activin A and/or activin B signaling as determined using a reporter gene assay.

26. The fusion protein, homodimer or heterodimer of claim 25, wherein the ActRIIB polypeptide further inhibits GDF8 and/or GDF11 signaling as determined using a reporter gene assay.

27. The fusion protein, homodimer or heterodimer of any preceding claim, wherein the activin antagonist domain is an ActRIIB polypeptide.

28. The fusion protein, homodimer or heterodimer of claim 27, wherein the ActRIIB polypeptide is selected from the group consisting of:
a. a polypeptide comprising an amino acid sequence that is at least 75% identical to a sequence beginning at any one of positions 20 to 29 of SEQ ID NO: 50, and ending at any one of positions 109 to 134 of SEQ ID NO: 50;
b. a polypeptide comprising an amino acid sequence that is at least 75% identical to a sequence beginning at position 20 of SEQ ID NO: 50, and ending at position 134 of SEQ ID NO: 50;
c. a polypeptide comprising an amino acid sequence that is at least 75% identical to a sequence beginning at position 29 of SEQ ID NO: 50, and ending at position 109 of SEQ ID NO: 50;
d. a polypeptide comprising an amino acid sequence that is at least 75% identical to SEQ ID NO: 51;
e. a polypeptide comprising an amino acid sequence that is at least 75% identical to SEQ ID NO: 52;
f. a polypeptide comprising an amino acid sequence that is at least 75% identical to SEQ ID NO: 54; and
g. a polypeptide comprising an amino acid sequence that is at least 75% identical to SEQ ID NO: 55.

29. The fusion protein, homodimer or heterodimer of claim 27 or 28, wherein the ActRIIB polypeptide binds to activin.

30. The fusion protein, homodimer or heterodimer of claim 29, wherein the ActRIIB polypeptide binds to activin A.

31. The fusion protein, homodimer or heterodimer of claim 29 or 30, wherein the ActRIIB polypeptide binds to activin B.

32. The fusion protein, homodimer or heterodimer of any one claims 29-31, wherein the ActRIIB polypeptide further binds to GDF8 and/or GDF11.

33. The fusion protein, homodimer or heterodimer of any one of claims 27-32, wherein the ActRIIB polypeptide inhibits activin A and/or activin B signaling as determined using a reporter gene assay.

34. The fusion protein, homodimer or heterodimer of claim 33, wherein the ActRIIB polypeptide further inhibits GDF8 and/or GDF11 signaling as determined using a reporter gene assay.

35. The fusion protein, homodimer or heterodimer of any preceding claim, wherein the activin antagonist domain is an antibody, or antigen-binding fragment thereof, that binds to activin.

36. The fusion protein, homodimer or heterodimer of claim 35, wherein the antibody, or antigen-binding fragment thereof, binds to activin A.

37. The fusion protein, homodimer or heterodimer of claim 35 or claim 36, wherein the antibody, or antigen-binding fragment thereof, binds to activin B.

38. The fusion protein, homodimer or heterodimer of any one of claims 35-37, wherein the antibody inhibits activin A and/or activin B signaling as determined using a reporter gene assay.

39. The fusion protein, homodimer or heterodimer of any preceding claim, wherein the activin antagonist domain is an antibody, or antigen-binding fragment thereof, that binds to an ActRII receptor.

40. The fusion protein, homodimer or heterodimer of claim 39, wherein the antibody, or antigen-binding fragment thereof, binds to ActRIIA.

41. The fusion protein, homodimer or heterodimer of claim 39 or 40, wherein the antibody, or antigen-binding fragment thereof, binds to ActRIIB.

42. The fusion protein, homodimer or heterodimer of any one of claims 39-41, wherein the antibody, or antigen-binding fragment thereof, inhibits activin-ActRIIA and/or activin-ActRIIB signaling as determined using a reporter gene assay.

43. The fusion protein, homodimer or heterodimer of claim 39, wherein the antibody, or antigen-binding fragment thereof is bimagrumab, or an antigen-binding fragment thereof.

44. The fusion protein, homodimer or heterodimer of any preceding claim, wherein the TGFβ antagonist domain is a TβRII polypeptide.

45. The fusion protein, homodimer or heterodimer of claim 44, wherein the TβRII polypeptide is selected from the group consisting of:
a. a polypeptide comprising an amino acid sequence that is at least 75% identical to a sequence beginning at any one of positions 23 to 35 of SEQ ID NO: 1, and ending at any one of positions 153 to 159 of SEQ ID NO: 1;
b. a polypeptide comprising an amino acid sequence that is at least 75% identical to a sequence beginning at position 23 of SEQ ID NO: 1, and ending at position 159 of SEQ ID NO: 1;
c. a polypeptide comprising an amino acid sequence that is at least 75% identical to a sequence beginning at position 35 of SEQ ID NO: 1, and ending at position 153 of SEQ ID NO: 1;
d. a polypeptide comprising an amino acid sequence that is at least 75% identical to a sequence beginning at any one of positions 23 to 60 of SEQ ID NO: 2, and ending at any one of positions 178 to 184 of SEQ ID NO: 2;
e. a polypeptide comprising an amino acid sequence that is at least 75% identical to a sequence beginning at position 23 of SEQ ID NO: 2, and ending at position 184 of SEQ ID NO: 2;
f. a polypeptide comprising an amino acid sequence that is at least 75% identical to a sequence beginning at position 60 of SEQ ID NO: 2, and ending at position 178 of SEQ ID NO: 2;
g. a polypeptide comprising an amino acid sequence that is at least 75% identical to SEQ ID NO: 18;
h. a polypeptide comprising an amino acid sequence that is at least 75% identical to SEQ ID NO: 27;
i. a polypeptide comprising an amino acid sequence that is at least 75% identical to SEQ ID NO: 20; and
j. a polypeptide comprising an amino acid sequence that is at least 75% identical to any one of SEQ ID NOs: 28, 29, 30, 31, 32, 33, 34, 35, 36, 37, 38; and 39.

46. The fusion protein, homodimer or heterodimer of claim 44 or 45, wherein the polypeptide binds to TGFβ3.

47. The fusion protein, homodimer or heterodimer of claim 46, wherein the polypeptide binds to TGFβ1.

48. The fusion protein, homodimer or heterodimer of claim 46 or 47, wherein the polypeptide binds to TGFβ3.

49. The fusion protein, homodimer or heterodimer of any one of claims 46-48, wherein the polypeptide inhibits TGFβ1 and/or TGFβ3 signaling as determined using a reporter gene assay.

50. The fusion protein, homodimer or heterodimer of any preceding claim, wherein the TGFβ antagonist domain is a betaglycan polypeptide.

51. The fusion protein, homodimer or heterodimer of claim 50, wherein the betaglycan polypeptide is selected from the group consisting of:
a. a polypeptide comprising an amino acid sequence that is at least 75% identical to a sequence beginning at any one of positions 21 to 28 of SEQ ID NO: 120, and ending at any one of positions 381 to 787 of SEQ ID NO: 120;
b. a polypeptide comprising an amino acid sequence that is at least 75% identical to a sequence beginning at position 21 of SEQ ID NO: 120, and ending at position 787 of SEQ ID NO: 120;
c. a polypeptide comprising an amino acid sequence that is at least 75% identical to a sequence beginning at position 28 of SEQ ID NO: 120, and ending at position 381 of SEQ ID NO: 120;
d. a polypeptide comprising an amino acid sequence that is at least 75% identical to SEQ ID NO: 121; and
e. a polypeptide comprising an amino acid sequence that is at least 75% identical to SEQ ID NO: 125.

52. The fusion protein, homodimer or heterodimer of claim 50 or 51, wherein the polypeptide binds to TGFβ3.

53. The fusion protein, homodimer or heterodimer of claim 52, wherein the polypeptide binds to TGFβ1.

54. The fusion protein, homodimer or heterodimer of claim 51 or 52, wherein the polypeptide binds to TGFβ2.

55. The fusion protein, homodimer or heterodimer of any one of claims 51-54, wherein the polypeptide binds to TGFβ3.

56. The fusion protein, homodimer or heterodimer of any one of claims 50-55, wherein the polypeptide inhibits TGFβ1, TGFβ2, and/or TGFβ3 signaling as determined using a reporter gene assay.

57. The fusion protein, homodimer or heterodimer of any preceding claim, wherein the TGFβ antagonist domain is an antibody, or antigen-binding fragment thereof, that binds to TGFβ.

58. The fusion protein, homodimer or heterodimer of claim 57, wherein the antibody, or antigen-binding fragment thereof, binds to TGFβ1.

59. The fusion protein, homodimer or heterodimer of claim 57 or claim 58, wherein the antibody, or antigen-binding fragment thereof, binds to TGFβ2.

60. The fusion protein, homodimer or heterodimer of any one of claims 57-59, wherein the antibody, or antigen-binding fragment thereof, binds to TGFβ3.

61. The fusion protein, homodimer or heterodimer of claim 57, wherein the antibody, or antigen-binding fragment thereof is selected from the antibodies: fresolimumab, metelimumab, Lily21D1, LilyDM4, XOMA089, and XOMA681, or an antigen-binding fragment thereof.

62. The fusion protein, homodimer or heterodimer of any one of claims 57-61, wherein the antibody inhibits TGFβ1, TGFβ2, and/or TGFβ3 signaling as determined using a reporter gene assay.

63. The fusion protein, homodimer or heterodimer of any preceding claim, wherein the heterologous portion comprises a first or second member of an interaction pair.

64. The fusion protein, homodimer or heterodimer of claim 63, wherein the heterologous portion comprises one or more amino acid modifications that promotes heterodimer formation.

65. The fusion protein, homodimer or heterodimer of any preceding claim, wherein the heterologous portion is an immunoglobulin Fc domain.

66. The fusion protein, homodimer or heterodimer of claim 65, wherein the immunoglobulin Fc domain is a human immunoglobulin Fc domain.

67. The fusion protein, homodimer or heterodimer of claim 65 or 66, wherein the immunoglobulin Fc domain is an immunoglobulin G1Fc domain.

68. The fusion protein, homodimer or heterodimer of any preceding claim, wherein the linker is between 10 and 25 amino acids in length.

69. The fusion protein, homodimer or heterodimer of any preceding claim, wherein the linker comprises an amino acid sequence selected from:
a. (GGGGS)ₙ, wherein n=≥2;
b. (GGGGS)ₙ, wherein n=≥3;
c. (GGGGS)ₙ, wherein n=≥4; and
d. the amino acid sequence of any one of SEQ ID Nos: 4-7, 19, 21, 25, 26, 40, and 63-67.

70. The heteromultimer of claim 69, wherein the linker comprises (GGGGS)ₙ, wherein n≠≥5.

71. The fusion protein, homodimer or heterodimer of claim 69, wherein the linker comprises (GGGGS)ₙ, wherein n≠≥5.

72. The fusion protein, homodimer or heterodimer of any preceding claim, wherein the fusion protein, homodimer or heterodimer comprises one or more modified amino acid residues selected from: a glycosylated amino acid, a PEGylated amino acid, a farnesylated amino acid, an acetylated amino acid, a biotinylated amino acid, and an amino acid conjugated to a lipid moiety.

73. The fusion protein, homodimer or heterodimer of claim 65, wherein the fusion protein, homodimer or heterodimer is glycosylated.

74. The fusion protein, homodimer or heterodimer of claim 73, wherein the fusion protein, homodimer or heterodimer has a glycosylation pattern characteristic of expression of the polypeptide in CHO cells.

75. The fusion protein, homodimer or heterodimer of any preceding claim, wherein the fusion protein, homodimer or heterodimer is isolated.

76. The fusion protein, homodimer or heterodimer of any preceding claim, wherein the fusion protein, homodimer or heterodimer is recombinant.

77. The fusion protein, homodimer, or heterodimer of any preceding claim, wherein the immune checkpoint antagonist domain inhibits one or more of PD-1, PD-L1, CTLA4, BTLA, LAG3, TIM3, LAIR1, B7-DC, HVEM, TIM4, B7-H3, and/or B7-H4.

78. The fusion protein, homodimer, or heterodimer of claim 77, wherein the immune checkpoint antagonist domain inhibits PD-1.

79. The fusion protein, homodimer, or heterodimer of claim 77, wherein the immune checkpoint antagonist domain inhibits PD-L1.

80. The fusion protein, homodimer, or heterodimer of claim 77, wherein the immune checkpoint antagonist domain inhibits CTLA-4.

81. The fusion protein, homodimer, or heterodimer of any preceding claim, wherein the immune checkpoint antagonist domain is an antibody, or antigen-binding fragment thereof, that binds to one or more of PD-1, PD-L1, CTLA4, BTLA, LAG3, TIM3, LAIR1, B7-DC, HVEM, TIM4, B7-H3, and/or B7-H4.

82. The fusion protein, homodimer, or heterodimer of claim 81, wherein the immune checkpoint antagonist domain is an antibody, or antigen-binding fragment thereof, that binds to PD-1.

83. The fusion protein, homodimer, or heterodimer of claim 81, wherein the immune checkpoint antagonist domain is an antibody, or antigen-binding fragment thereof, that binds to PD-L1.

84. The fusion protein, homodimer, or heterodimer of claim 81, wherein the immune checkpoint antagonist domain is an antibody, or antigen-binding fragment thereof, that binds to CTLA4.

85. The fusion protein, homodimer, or heterodimer of any preceding claim, wherein the immune checkpoint antagonist domain is an antibody selected from ipilimumab, nivolumab, pembrolizumab, atezolizumab, avelumab, and durvalumab, or antigen-binding fragment thereof.

86. A pharmaceutical preparation comprising the fusion protein, homodimer or heterodimer of any preceding claim and a pharmaceutically acceptable excipient.

87. An isolated polynucleotide comprising a coding sequence for the fusion protein, homodimer or heterodimer of any preceding claim.

88. A recombinant polynucleotide comprising a promotor sequence operably linked to the polynucleotide of claim 87.

89. A cell comprising the polynucleotide of claim 87 or 88.

90. The cell of claim 89, wherein the cell is a CHO cell.

91. A method of making a fusion protein, homodimer or heterodimer comprising two or more domains selected from an activin antagonist domain, a TGFβ antagonist domain comprising culturing a cell under conditions suitable for expression of the polynucleotide of claim 88 or 88.

92. A method of treating cancer, a tumor, a pre-neoplastic disorder, a hyperproliferative disorder, or a dysplastic disorder comprising administering to a patient in need thereof an effective amount of one or more of the fusion proteins, homodimers, heterodimers or pharmaceutical preparations of any one of claims 1-86.

93. The method of claim 92, wherein the cancer, tumor, pre-neoplastic disorder, hyperproliferative disorder, or dysplastic disorder is selected from the group consisting of: a hematopoietic tumor of lymphoid or myeloid lineage, a tumor of mesenchymal origin such as a fibrosarcoma or rhabdomyosarcoma, melanoma, intraocular melanoma, nonmelanoma skin cancer, teratocarci-noma, neuroblastoma, glioma, brain stem glioma, visual pathway and hypothalamic glioma, oligodendroglioma, adenocarcinoma, papillary adenocarcinomas, cystadenocarcinoma, carcinoma, non-small lung cell carcinoma, hepatoma, hepatocellular carcinoma, endometrial cancer or uterine carcinoma, salivary gland carcinoma, differentiated thyroid carcinoma, carcinoma of the lung, penile carcinoma, adrenocortical carcinoma, endocrine pancreas islet cell carcinoma, colon carcinoma, squamous cell carcinoma, basal cell carcinoma, sweat gland carcinoma, sebaceous gland carcinoma, papillary carcinoma, medullary carcinoma, bronchogenic carcinoma, renal cell carcinoma, anal carcinoma, bile duct carcinoma, choriocarcinoma, embryonal carcinoma, epithelial carcinoma, lymphoma, adult Hodgkin's lymphoma, adult non-Hodgkin's lymphoma, AIDS-related lymphoma, central nervous system lymphoma, cutaneous T-cell lymphoma, T-Cell lymphoma, seminoma, glioblastoma, glioblastoma multiforme, sarcoma, Ewing sarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, soft tissue sarcoma, Kaposi's sarcoma, osteo/malignant fibrous sarcoma, osteosarcoma/malignant fibrous histiocytoma, sarcoidosis sarcoma, uterine sarcoma, lymphangioendotheliosarcoma, leukemia, acute lymphoblastic leukemia, acute lymphocytic leukemia, acute myeloid leukemia, chronic lymphocytic leukemia, chronic myelogenous leukemia, hairy cell leukemia, myelogenous leukemia, myeloid leukemia, myeloblastic leukemia, promyelocytic leukemia, myelomonocytic leukemia, monocytic leukemia, a erythroleukemia, chronic myelocytic leukemia, leukemia myeloma, multiple myeloma, lymphoid malignancies, squamous cell cancer, epithelial squamous cell cancer, squamous cancer of the peritoneum, squamous neck cancer, metastatic squamous neck cancer, metastatic squamous neck cancer, occult metastatic squamous neck cancer, Wilms tumor, astrocytomas, lung cancer, small-cell lung cancer, non-small cell lung cancer, hepatocellular cancer, gastric or stomach cancer, gastrointestinal cancer, gastrointestinal carcinoid tumor, pancreatic cancer, exocrine pancreatic cancer, islet cell pancreatic cancer, cervical cancer, cervical dysplasia, ovarian cancer, ovarian epithelial cancer, ovarian germ cell tumor, ovarian low malignant potential tumor, liver cancer, neuroendocrine tumors, medullary thyroid cancer, parathyroid cancer, breast cancer, colon cancer, rectal cancer, kidney or renal cancer, prostate cancer, vulvar cancer, head-and-neck cancer, AIDS-related malignancies, anal cancer, astrocytoma, cerebellar astrocytoma, cerebral astrocytoma, bile duct cancer, extrahepatic bile duct cancer, bone cancer, fibrous dysplasia of bone, brain tumors, extracranial germ cell tumors, extragonadal germ cell tumor, germ cell tumors, Hodgkin's disease, medulloblastoma, pineal tumors, pinealoma, supratentorial neuroectodermal tumors, ependymoma, epithelial cancer, epithelial dysplasia, mucoepithelial dysplasia, esophageal cancer, esophageal dysplasia, eye cancer, Gaucher's disease, gallbladder cancer, gestational TROPhoblastic tumor, TROPhoblastic tumors, hypergammaglobulinemia, hypopharyngeal cancer, intestinal cancers, intestinal polyps or adenomas, small intestine cancer, large intestine cancer, laryngeal cancer, lip or oral cavity cancer, lymphoproliferative disorders, macroglobulinemia, Waldenstrom's macroglobulinemia, mesothelioma, malignant thymoma, thymoma, metastatic occult plasma cell neoplasm, myelodysplastic syndrome, myeloproliferative disorders, nasal cavity or paranasal sinus cancer, nasopharyngeal cancer, oropharyngeal cancer, paraproteinemias, penile cancer, pheochromocytoma, pituitary tumor, retinoblastoma, salivary gland cancer, Sezary syndrome, skin cancer, testicular cancer, urethral cancer, uterine cancer, vaginal cancer, anhidrotic ectodermal dysplasia, anterofacial dysplasia, asphyxiating thoracic dysplasia, atriodigital dysplasia, bronchopulmonary dysplasia, cerebral dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, faciodigitogenital dysplasia, familial fibrous dysplasia of jaws, familial white folded dysplasia, fibromuscular dysplasia, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, ventriculoradial dysplasia, benign dysproliferative disorders (e.g., benign tumors, fibrocystic conditions, tissue hypertrophy, and), leukoplakia, keratoses, Bowen's disease, Farmer's skin, solar cheilitis, solar keratosis, heavy chain disease, synovioma, craniopharyngioma, emangioblastoma, acoustic neuroma, and meningioma.

94. The method of claim 92 or 93, wherein the method further comprises administration of one or more additional active agents or supportive therapies for treating the cancer, tumor, pre-neoplastic disorder, hyperproliferative disorder, or dysplastic disorder.

95. The method of claim 94, wherein the additional active agent or supportive therapy is an immune checkpoint antagonist, and wherein the immune checkpoint antagonist is selected from a polypeptide, an antibody or antigen-binding fragment thereof, a small molecule, and/or polynucleotide.

96. The method of claim 95, wherein the immune checkpoint antagonist is an antibody, or antigen-biding fragment thereof that binds to one or more of: PD-1, PD-L1, CTLA4, BTLA, LAG3, TIM3, LAIR1, B7-DC, HVEM, TIM4, B7-H3, and/or B7-H4.

97. The method of claim 96, wherein the antibody is ipilimumab, nivolumab, pembrolizumab, atezolizumab, avelumab, and durvalumab, or an antigen binding fragment thereof.

98. The method of any one of claims 95-97, wherein the additional active agent or supportive therapy is an activin antagonist, and wherein the activin antagonist is selected from a polypeptide, an antibody or antigen-binding fragment thereof, a small molecule, and/or polynucleotide.

99. The method of claim 98, wherein the activin antagonist is an ActRII polypeptide of any preceding claim.

100. The method of claim 98, wherein the activin antagonist is an ActRII antibody of any preceding claim.

101. The method of claim 98, wherein the activin antagonist is an activin antibody of any preceding claim.

102. The method of any one of claims 94-101, wherein the additional active agent or supportive therapy is a TGFβ antagonist, and wherein the TGFβ antagonist is selected from a polypeptide, an antibody or antigen-binding fragment thereof, a small molecule, and/or polynucleotide.

103. The method of claim 102, wherein the TGFβ antagonist is a TβRII polypeptide of any preceding claim.

104. The method of claim 102, wherein the TGFβ antagonist is an TGFβ antibody of any preceding claim.

105. The method of claim 102, wherein the TGFβ antagonist is a betaglycan polypeptide of any preceding claim.
